(19) United States
(12) Patent Application Publication
(10) Pub. No.: US 2017/0348263 A1
(43) Pub. Date: Dec. 7, 2017

(54) COMPOSITIONS AND METHODS OF USING MODIFIED RELEASE SOLABEGRON FOR LOWER URINARY TRACT SYMPTOMS

(71) Applicant: VELICEPT THERAPEUTICS, INC., MALVERN, PA (US)

(72) Inventors: Eliot OHLSTEIN, Glennmoore, PA (US); Raymond E. STEVENS, JR., West Chester, PA (US); H. Jeffrey WILKINS, Sellersville, PA (US)

(21) Appl. No.: 15/614,577

(22) Filed: Jun. 5, 2017

Related U.S. Application Data
(60) Provisional application No. 62/345,388, filed on Jun. 3, 2016.
(60) Provisional application No. 62/345,519, filed on Jun. 3, 2016.

(51) Int. Cl. A61K 31/196; A61K 9/20; A61K 9/00; A61K 9/16; A61K 9/50; A61K 9/24

(52) U.S. Cl. CPC A61K 31/196; A61K 9/5084; A61K 9/209; A61K 9/0053; A61K 9/1676; A61K 9/2081

(57) ABSTRACT

This application relates to pharmaceutical compositions, comprising solabegron that are useful for the treatment of lower urinary tract symptoms such as, for example, overactive bladder and prostate disorders. Additionally, this application relates to methods for treating lower urinary tract symptoms utilizing the pharmaceutical compositions, comprising solabegron. In some embodiments, the pharmaceutical compositions, comprising solabegron comprise a dual release drug delivery system.

The present application claims the benefit of U.S. Provisional Application No. 62/345,388, filed Jun. 3, 2016; and U.S. Provisional Application No. 62/345,519, filed Jun. 3, 2016; the disclosures of which are hereby incorporated by reference in their entirety.

### SUMMARY

Agonist-induced desensitization of G-protein coupled receptors (GPCRs) of the beta-3 adrenoceptor is not well studied. For many disease processes, GPCR desensitization is thought to contribute to the disease process or limit the effect of therapeutic agents. Prolonged exposure of the receptor system molecule to a drug may result in receptor down-regulation. Down-regulation occurs when there is a decrease in the number of receptor system molecules on the cell, thus decreasing the response to continued administration of the therapeutic agent. In addition, more drug may often be needed over time to achieve the same therapeutic response. Pharmaceutical compositions of beta-3 adrenoceptor agonists that can minimize desensitization would be expected to increase therapeutic response and therefore be beneficial in treating subjects when compared with pharmaceutical compositions that do not minimize desensitization. The present invention describes pharmaceutical compositions that increase the therapeutically effective properties of solabegron, while otherwise minimizing such desensitization and methods of using these pharmaceutical compositions for the treatment of diseases.

Various embodiments described herein relate to pharmaceutical compositions comprising a therapeutically effective amount of solabegron that achieves a first target Cₘₐₓ of solabegron, a second target Cₘₐₓ of solabegron, a first target Cₘᵢₙ of solabegron between the first target Cₘₐₓ and the second target Cₘₐₓ and a second Cₘᵢₙ of solabegron after the second target Cₘₐₓ. In embodiments, the pharmaceutical compositions reduce desensitization of the beta-3 adrenoceptor, particularly when compared to an immediate release formulation of solabegron that may be given, for example, twice daily.

In embodiments, the pharmaceutical compositions achieve a plasma concentration [C] of solabegron of about 1 μg/mL or below for a period of time of about 6 hours to about 9 hours during a 24 hour period. In embodiments, the pharmaceutical compositions achieve an AUC of about 5,000 ng.hr/mL to about 30,000 ng.hr/mL over a 24 hour period. In embodiments the pharmaceutical compositions are administered once a day to a subject in need thereof.

Further embodiments are directed to a pharmaceutical composition for the delivery of solabegron, comprising (a) at least one immediate release composition, comprising solabegron and at least one pharmaceutically acceptable carrier or diluent; and (b) at least one modified release composition, comprising solabegron and at least one pharmaceutically acceptable carrier or diluent. In some embodiments the pharmaceutical composition may have any of the Cₘₐₓ, Cₘᵢₙ, Tₘₐₓ, or Tₘᵢₙ described herein. Some embodiments herein are directed to a pharmaceutical composition comprising a multiparticulate formulation of mini-tablets each comprising a therapeutically effective amount of solabegron, wherein the pharmaceutical composition achieves a first target Cₘₐₓ, a second target Cₘₐₓ, a first target Cₘᵢₙ between the first target Cₘₐₓ and the second target Cₘₐₓ, and a second target Cₘᵢₙ after the second target Cₘₐₓ.

Some embodiments describe a pharmaceutical composition comprising a therapeutically effective amount of the amorphous form of solabegron and at least one pharmaceutically acceptable carrier or diluent.

Further embodiments are directed to the use of such pharmaceutical compositions for the treatment of diseases, including, but not limited to, lower urinary tract symptoms (hereinafter “TAUS”), obesity, type 2 diabetes, heart failure, irritable bowel syndrome and similar gastrointestinal disorders, pre-term labor, depression and anxiety. In embodiments, LUTS may be overactive bladder and/or prostate disorders.

Some embodiments describe a method of treating LUTS, obesity, type 2 diabetes, heart failure, irritable bowel syndrome, gastrointestinal disorders, pre-term labor, depression and anxiety in a subject in need thereof, comprising administering to the subject, a pharmaceutical composition for the delivery of solabegron, comprising an immediate release composition, comprising solabegron and at least one pharmaceutically acceptable carrier or diluent; and a modified release composition, comprising solabegron and at least one pharmaceutically acceptable carrier or diluent.

Some embodiments are directed to a method of treating LUTS, obesity, type 2 diabetes, heart failure, irritable bowel syndrome and similar gastrointestinal disorders, pre-term labor, depression and anxiety in a subject in need thereof, comprising administering a pharmaceutical composition to the subject, comprising a therapeutically effective amount of solabegron, wherein the pharmaceutical composition releases at least two releases of solabegron, wherein a first release of solabegron achieves a first target Cₘₐₓ, a second release of solabegron achieves a second target Cₘₐₓ, a first target Cₘᵢₙ is achieved between the first release and the second release and a second Cₘᵢₙ is achieved after the second release.

Some embodiments are directed to a method for treating overactive bladder in a patient in need thereof comprising orally administering once a day, to the patient, a pharmaceutical composition comprising: an immediate release drug layer comprising about 75 mg to about 250 mg solabegron and at least one pharmaceutically acceptable carrier or diluent; and a delayed release core comprising about 100 mg to about 400 mg solabegron and at least one pharmaceutically acceptable carrier or diluent, wherein the immediate release drug layer is coated on the delayed release core

### BRIEF DESCRIPTION OF THE FIGURES

FIG. 1—Graphical Illustration of a dual-release pharmaceutical composition that achieves a first target Cₘₐₓ, provides a period at a first target Cₘᵢₙ, achieves a second target Cₘₐₓ and finally provides a period at a second target Cₘᵢₙ.

FIG. 2—Cumulative concentration response curves (CCRC) to solabegron performed after a one hr incubation to the EC₉₀ concentration of solabegron and a period of washout using PSS. Two-way ANOVA to compare the curves gives p<0.001, with a Bonferroni post hoc test to compare individual points with comparable points on the vehicle incubation curve (black triangles). *=p<0.05, **=p<0.01, ***=p<0.001.

FIG. 3—Cumulative concentration response curves (CCRC) to solabegron performed after a three hr incubation to the EC₉₀ concentration of solabegron and a period of washout using PSS. Two-way ANOVA to compare the curves gives p<0.001, with a Bonferroni post hoc test to compare individual points with comparable points on the vehicle incubation curve (black triangles). *=p<0.05, **=p<0.01, ***=p<0.001.

FIG. 4—Cumulative concentration response curves (CCRC) to CL-316,243 performed after a three hr incubation to the EC₉₀ concentration of CL-316,243 and a period of washout using PSS. Two-way ANOVA to compare the curves gives p<0.001, with a Bonferroni post hoc test to compare individual points with comparable points on the vehicle incubation curve (black triangles). *=p<0.05, **=p<0.01, ***=p<0.001.

FIG. 5—Graphical illustration of a tablet that is an immediate release formulation and a tablet that is a modified release formulation enclosed in a capsule.

FIG. 6—Graphical illustration of a dissolution study of solabegron-polymer formulation plotted as a concentration of solabegron in solution as a function of time.

FIG. 7—Fourier-transform infrared spectroscopy (FTIR) scans of solabegron API, a spray-dried 20:80 solabegron to polyvinyl pyrrolidine (PVPK30) formulation, a physical mixture of 20:80 solabegron to polyvinyl pyrrolidine (PVPK30) and the PVPK30 carrier. The solabegron API displays amine (N—H) peaks at about 3400 and 3250 cm⁻¹ and carbonyl peaks at 1593 cm⁻¹. The PVPK30 carrier displays a carbonyl peak at about 1670 cm⁻¹. The observed changes in the amine and carbonyl peaks in the solabegron-PVPK30 formulation is absent in the physical mixture. The observed changes are indicative of the amorphous form of solabegron.

FIG. 8—Graphical illustration of a tablet that is a modified release solabegron core (solabegron with a modified (MR) release coating), that is coated in a matrix of immediate/early release solabegron and a polymer.

FIG. 9—Graphical illustration of a dissolution study of solabegron composition A (See Example 22) plotted as a percent of solabegron in solution as a function of time.

FIG. 10—Graphical illustration of a dissolution study of solabegron composition B (See Example 22) plotted as a percent of solabegron in solution as a function of time.

FIG. 11—Graphical illustration of a dissolution study of solabegron composition C (See Example 22) plotted as a percent of solabegron in solution as a function of time.

FIG. 12—Graphical illustration of a dissolution study of solabegron composition E (See Example 22) plotted as a percent of solabegron in solution as a function of time.

FIG. 13—Graphical illustration of a dissolution study of 75 mg immediate release solabegron coated delayed release placebo core in 500 mL of FaSSGF (See Example 22) plotted as a percent of solabegron in solution as a function of time.

FIG. 14—Graphical illustration of a dissolution study of solabegron composition A (See Example 23) plotted as a percent of solabegron in solution as a function of time.

FIG. 15—Graphical illustration of a dissolution study of solabegron composition B (See Example 23) plotted as a percent of solabegron in solution as a function of time.

FIG. 16—Graphical illustration of a dissolution study of solabegron composition C (See Example 23) plotted as a percent of solabegron in solution as a function of time.

FIG. 17—Graphical illustration of a dissolution study of solabegron composition D (See Example 23) plotted as a percent of solabegron in solution as a function of time.

FIG. 18—Graphical illustration of immediate release/modified release PK profile for 50 mg immediate release solabegron combined with 200 mg modified release solabegron plotted as ng/mL as a function of time.

FIG. 19—Graphical illustration of immediate release/modified release PK profile for interpolated 75 mg immediate release solabegron combined with 200 mg modified release solabegron plotted as ng/mL as a function of time.

FIG. 20—Graphical illustration of immediate release/modified release PK profile for 100 mg immediate release solabegron combined with 200 mg modified release solabegron plotted as ng/mL as a function of time.

FIG. 21—Graphical illustration of immediate release/modified release PK profile for interpolated 125 mg immediate release solabegron combined with 200 mg modified release solabegron plotted as ng/mL as a function of time.

FIG. 22—Graphical illustration of immediate release/modified release PK profile for 150 mg immediate release solabegron combined with 200 mg modified release solabegron plotted as ng/mL as a function of time.

FIG. 23—Table representing time and events for screening through end of the study described in Example 25 by study day.

FIG. 24—Table representing time and events for screening before and after dosing in the study described in Example 25 by hour.

FIG. 25—Graphical illustration of the PK data for the solabegron 200 mg total dose DR-4 from Example 25 plotted as ng/mL of solabegron as a function of time.

FIG. 26—Graphical illustration of the PK data for the solabegron 200 mg total dose DR-4 from Example 25 plotted as ng/mL of solabegron as a function of time in log scale.

### DETAILED DESCRIPTION

Solabegron (3′-[(2-{[(2R)-2-(3-chlorophenyl)-2-hydroxyethyl]amino}ethypamino]biphenyl-3-carboxylic acid) is a beta-3 adrenoceptor agonist, with the following structure:

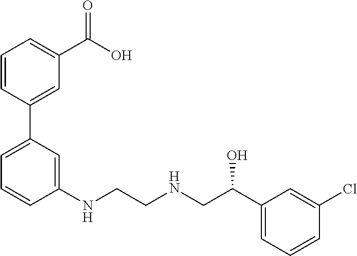 [SMILES: O=C(O)c1cccc(-c2cccc(NCCNC[C@H](O)c3cccc(Cl)c3)c2)c1]

It is further described in U.S. Pat. No. 6,251,925, U.S. Pat. No. 8,642,661 and United States Patent Publication No. 2013/0172277A1 (now U.S. Pat. No. 9,522,129), PCT Application No. US2015/38583 filed Jun. 30, 2015; PCT Application No. PCT/US2015/63795, and PCT Application No. US2016/058516. Solabegron has been demonstrated to significantly reduce the symptoms of overactive bladder (hereinafter “OAB”) in women with moderate to severe OAB, showing that solabegron is safe, well-tolerated, and does not demonstrate significant differences in adverse events as compared to placebo.

The use of a beta-3 adrenoceptor agonist may be limited by beta-3 receptor desensitization. It is conceivable that like the beta-2 adrenoceptor in airway smooth muscle, continuous, prolonged administration of a beta-3 adrenoceptor agonist will elicit beta-3 receptor desensitization in bladder smooth muscle. Prolonged exposure of a beta-3 adrenoceptor agonist may possibly result in a decrease in the number of beta-3 receptors, a decrease binding affinity or diminish post-receptor signal transduction mechanisms and second messenger signaling, resulting in a diminished therapeutic response.

To prevent or reduce beta-3 adrenoceptor desensitization, it is described herein that the therapeutic administration of a beta-3 adrenoceptor agonist occurs in a manner such that drug occupancy at the receptor occurs at levels that do not elicit significant receptor desensitization and pharmaceutical compositions that achieve the same.

It is well established in the GPCR field that prolonged occupancy of a receptor by an agonist can result in receptor desensitization. A method to limit this is to have the agonist off the receptor, and allow the receptor to recover from agonist occupancy. When examining an entire population of receptors, the entire population of receptors does not need to be unoccupied; fractional occupancy of the entire receptor population can still result in prevention of receptor desensitization and preservation of function. In other words, anything lower than 100% receptor occupancy may still allow some percentage of receptor resensitization.

The pharmaceutical compositions and methods of administration as described herein will not produce significant receptor desensitization, while ensuring the method of administration will optimize for the beta-3 adrenoceptor stimulation, thus enabling the target tissue to benefit fully from the administered therapeutic agent. The therapeutic agent, in the present application, is the beta-3 adrenoceptor agonist solabegron The pharmaceutical compositions may have a selected amplitude and duration so that the beta-3 adrenoceptor will not down-regulate and the binding affinity of the receptor system molecule will not be diminished.

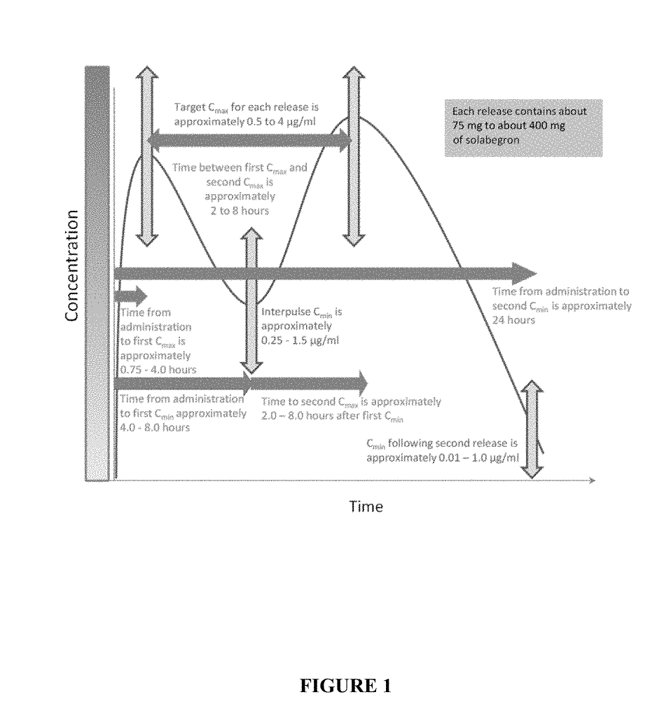

Embodiments of the present application describe pharmaceutical compositions comprising a therapeutically effective amount of solabegron in a succession of at least two releases, wherein each release is optimized to provide a therapeutically effective plasma concentration [C] that optimizes the tissue response while also providing a lower plasma concentration [C] between the first and second release as well as between the second release and the subsequent administration of the pharmaceutical composition to allow for a sufficient recovery time for the beta-3 adrenoceptors and methods of using the same to treat diseases. An exemplary embodiment of such a pharmaceutical composition and its release profile is provided in (FIG. 1).

Administration of a drug to treat such disease could be by the oral or parenteral routes of administration. For the oral route of administration, a pharmaceutical composition is described that releases drug for systemic absorption at the desired time-points and releases the desired systemic plasma drug levels.

### Definitions

As used herein, the term “about” means plus or minus 10% of a given value. For example, “about 50%” means in the range of 45% -55%.

As used herein the term “agonist” refers to a compound, the presence of which results in a biological activity of a receptor that is the same as the biological activity resulting from the presence of a naturally occurring ligand for the receptor.

The terms “amorphous form”, amorphous solid” and amorphous solabegron” refer to a solabegron solid that does not have a definite geometric or crystalline shape. It is a solid in which there is no long-range order in the positions of the atoms that is characteristic or a crystal.

As used herein the terms “area under the curve” and “AUC” is the area under the curve (mathematically known as a definite integral) in a pharmacokinetic plot of the concentration of a drug against time.

As used herein the terms “BID” and “b.i.d.” mean twice a day (from the Latin bis in die).

As used herein the terms “Cₘₐₓ”, “Cₘᵢₙ”, “Tₘₐₓ”, and “T_{1/2}” are terms used in pharmacokinetic analyses of the concentration of a drug against time. Cₘₐₓ is a term that refers to the maximum (or peak) plasma concentration that a drug achieves in a specified compartment or test area of the body after the drug has been administrated and prior to the administration of a second dose. Cₘₐₓ is the opposite of Cₘᵢₙ, which is the minimum (or trough) concentration that a drug achieves after dosing. Tₘₐₓ is the term used in pharmacokinetics to describe the time at which the Cₘₐₓ is observed and Tₘᵢₙ is the term used in pharmacokinetics to describe the time at which the Cₘᵢₙ is observed after the drug has been administered and prior to the administration of a second dose. T_{1/2} is the time it takes for the peak plasma concentration to reach half of its original value after administration to a subject.

The term “delayed release” as used herein is a dosage form that releases a drug at a time other than immediately upon administration.

As used herein, the term “desensitization” refers to a state wherein a receptor, specifically in the present application a beta-3 adrenoceptor, has been overexposed to an agonist for an extended period of time and an increased dosage of agonist must be administered to achieve a similar physiological response. It is a process whereby after prolonged agonist exposure, the receptor is uncoupled from its signaling cascade, and thus the biological effect of receptor activation is attenuated. Desensitization occurs when the beta-3 adrenoceptor is not otherwise responsive to an agonist (or antagonist), is less responsive to an agonist (or antagonist), or the target tissue (e.g., the bladder) is not otherwise responsive or is less responsive to an agonist (or antagonist).

As used herein the phrase “drug delivery system” refers to any physical form, vehicle or composition that may be formulated to administer a therapeutic agent to a subject in need thereof such as, for example but not limited to the following: tablets, capsules, granules, powders, liquids, suspensions, suppositories, ointments, creams and aerosols.

As used herein, the term “effective amount” refers to an amount that results in measurable inhibition of at least one symptom or parameter of a specific disorder or pathological process.

The term “extended release” or “sustained release” as used herein is a dosage form that makes a drug available over an extended period of time afteradministration.

As used herein the term “immediate release” refers to pharmaceutical compositions that release the active ingredient within a short period of time, typically less than 30 minutes.

As used herein the phrase “lower urinary tract symptoms” or “LUTS” refers to a group of medical symptoms comprising increased frequency of urination, increased urinary urgency of urination, painful urination, excessive passage of urine at night (nocturia), poor stream, overactive bladder, hesitancy, terminal dribbling, incomplete voiding, and overflow incontinence. Subjects with LUTS may have one or more of these symptoms.

As used herein the term “modified release” refers to pharmaceutical compositions that does not otherwise release the active ingredient immediately, for example it may release the active ingredient at a sustained or controlled rate over an extended period of time such as, for example, 4 hours, 8 hours, 12 hours, 16 hours, and 24 hours or release the pharmaceutical dosage after a set time such as, for example, enteric-coated compositions that release the dosage in the intestinal track. Modified release includes, extended release, sustained release and delayed release.

As used herein the phrase “overactive bladder” or “OAB” refers to a group of medical symptoms comprising urinary urgency, frequent urination, nocturia, urinating unintentionally and urge incontinence. Subjects with OAB may have one or more of these symptoms.

The phrase “pharmaceutically acceptable” refers to molecular entities and compositions that are generally regarded as safe and nontoxic. In particular, pharmaceutically acceptable carriers, diluents or other excipients used in the pharmaceutical compositions of this application are physiologically tolerable, compatible with other ingredients, and do not typically produce an allergic or similar untoward reaction (e.g., gastric upset, dizziness and the like) when administered to a subject. Preferably, as used herein, the term “pharmaceutically acceptable” means approved by a regulatory agency of the Federal or a state government or listed in the U.S. Pharmacopoeia or other generally recognized pharmacopoeia for use in animals, and more particularly in humans.

The phrase “pharmaceutically acceptable salt(s)”, as used herein, includes those salts of compounds of the application that are safe and effective for use in mammals and that possess the desired biological activity. Pharmaceutically acceptable salts include salts of acidic or basic groups present in compounds of the application or in compounds identified pursuant to the methods of the application. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the application can form pharmaceutically acceptable salts with various amino acids. Suitable base salts include, but are not limited to, aluminum, calcium, lithium, magnesium, potassium, sodium, zinc, iron and diethanolamine salts. Pharmaceutically acceptable base addition salts are also formed with amines, such as organic amines. Examples of suitable amines are N,N′-dibenzylethylenediamine, chloroprocaine, choline, diethanolamine, dicyclohexylamine, ethylenediamine, N-methylglucamine, and procaine.

As used herein the terms “QD” and “q.d.” mean once a day (from the Latin quaque die).

As used herein the terms, “release”, “releases”, “delivery”, “pulsatile delivery device”, refer to pharmaceutical compositions and methods of treatment wherein a therapeutic agent is delivered rapidly within a short, predetermined period of time, as a result of a biological or external trigger or after a specific lag time The terms can also refer to pharmaceutical compositions and methods of treatment wherein a therapeutic agent is delivered within a predetermined period of time.

The term solabegron refers to (3′-[(2-{[(2R)-2-(3-chlorophenyl)-2-hydroxyethyl]amino}ethyl)amino]biphenyl-3-carboxylic acid) is a beta-3 adrenoceptor agonist, with the following structure:

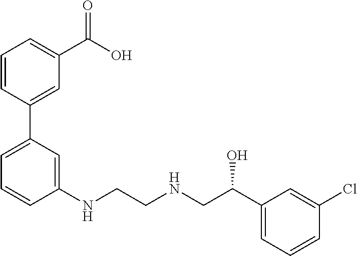 [SMILES: O=C(O)c1cccc(-c2cccc(NCCNC[C@H](O)c3cccc(Cl)c3)c2)c1]

Solabegron is solabegron or a pharmaceutically acceptable salt thereof In embodiments, solabegron is amorphous, zwitterion or the free base. In embodiments, a pharmaceutically acceptable salt thereof may include, but is not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)), various amino acids, aluminum, calcium, lithium, magnesium, potassium, sodium, zinc, iron, diethanolamine, amines, such as organic amines, N,N′-dibenzyl ethylenediamine, chloroprocaine, choline, diethanolamine, dicyclohexylamine, ethylenediamine, N-methylglucamine, and procaine. Solabegron may exist in any physical form known to one of skill in the art such as, for example, nanoparticles, crystalline solids, amorphous solids, polymorphs, ionic solids such as, for example, cations, anions and zwitterions, pharmaceutically acceptable salts, hydrates, solvates, stereoisomers, solutions and suspensions. Crystalline solids have regular ordered arrays of components held together by uniform intermolecular forces, whereas the components of amorphous solids are not arranged in regular arrays. Hydrates are substances that incorporate at least one water molecule into their crystalline matrix. Solvates are substances that incorporate at least one solvent molecule into their crystalline matrix. Polymorphs exhibit different crystalline structures for molecules that have the same molecular formula and sequence of bonded atoms. Stereoisomers are isomeric molecules that have the same molecular formula and sequence of bonded atoms (constitution), but that differ only in the three-dimensional orientations of their atoms in space. In some embodiment solabegron is the amorphous solid form of solabegron. In some embodiments, solabegron is solabegron hydrochloride. In some embodiments the solabegron is the zwitterion form of solabegron.

The terms “subject,” “individual” or “patient” are used interchangeably and as used herein are intended to include human and non-human animals. Non-human animals includes all vertebrates, e.g. mammals and non-mammals, such as non-human primates, sheep, dogs, cats, cows, horses, chickens, amphibians, and reptiles, although mammals are preferred, such as non-human primates, sheep, dogs, cats, cows and horses. Preferred subjects include humans in need of treatment. The methods are particularly suitable for treating humans having a disease or disorder described herein.

As used herein, the term “therapeutic” means an agent utilized to treat, combat, ameliorate, protect against or improve an unwanted condition or disease of a subject.

The term “therapeutic benefit” means having an effect that results in a beneficial outcome. For example, the beneficial outcome can be a minimize desensitization of the beta-3 adrenoceptor; an increase therapeutic response; amelioration, protection against or improvement of an unwanted condition or disease; alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; and remission of a disorder or disease. The therapeutic benefit may be objective (i.e., measurable by some test or marker) or subjective (i.e., subject gives an indication of or feels an effect or physician observes a change).

As used herein the term “therapeutically effective amount” of compositions of the application is an amount, which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect or physician observes a change).

As used herein the terms “TID” and t.i.d.” mean three times a day (from the Latin ter in die).

As used herein the terms “treat”, “treated”, or “treating” refer to both therapeutic treatment and prophylactic or preventative measures, wherein the object is to protect against (partially or wholly) or slow down (e.g., lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this application, beneficial or desired clinical results include, but are not limited to, alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects.

### Pharmaceutical Compositions

In one embodiment, the present application describes a pharmaceutical composition comprising a therapeutically effective amount of solabegron, wherein the pharmaceutical composition achieves a first target Cₘₐₓ, a second target Cₘₐₓ, a first target Cₘᵢₙ between the first target Cₘₐₓ and the second target Cₘₐₓ, and a second target Cₘᵢₙ after the second target Cₘₐₓ.

In embodiments, the first target Cₘₐₓ is about 0.5 μg/mL to about 4.0 μg/mL. In embodiments, the first target Cₘₐₓ is about 1.0 μg/mL to about 3.5 μg/mL. In embodiments, the first target Cₘₐₓ is about 1 μg/mL to about 2.0 μg/mL. In embodiments, the first target Cₘₐₓ is about 1.5 μg/mL to about 3.5 μg/mL. In embodiments, the first target Cₘₐₓ is about 1.5 μg/mL to about 3.0 μg/mL. In embodiments, the first target Cₘₐₓ is about 2.0 μg/mL to about 3.5 μg/mL. In embodiments, the first target Cₘₐₓ is about 2.0 μg/mL to about 3.0 μg/mL. In embodiments, the first target Cₘₐₓ is about 1.0 μg/mL to about 4.0 μg/mL. In embodiments, the first target Cₘₐₓ is about 1.5 μg/mL to about 4.0 μg/mL. In embodiments, the first target Cₘₐₓ is about 2.0 μg/mL to about 4.0 μg/mL. In embodiments, the first target Cₘₐₓ is about 2.5 μg/mL to about 4.0 μg/mL. In embodiments, the first target Cₘₐₓ is about 3.0 μg/mL to about 4.0 μg/mL. In embodiments, the first target Cₘₐₓ is about 3.5 μg/mL to about 4.0 μg/mL. In embodiments, the first target Cₘₐₓ is about 0.5 μg/mL to about 3.5 μg/mL. In embodiments, the first target Cₘₐₓ is about 2.5 μg/mL to about 3.5 μg/mL. In embodiments, the first target Cₘₐₓ is about 3.0 μg/mL to about 3.5 μg/mL. In embodiments, the first target Cₘₐₓ is about 0.5 μg/mL to about 3.0 μg/mL. In embodiments, the first target Cₘₐₓ is about 1.0 μg/mL to about 3.0 μg/mL. In embodiments, the first target Cₘₐₓ is about 2.5 μg/mL to about 3.0 μg/mL. In embodiments, the first target Cₘₐₓ is about 0.5 μg/mL to about 2.5 μg/mL. In embodiments, the first target Cₘₐₓ is about 1.0 μg/mL to about 2.5 μg/mL. In embodiments, the first target Cₘₐₓ is about 1.5 μg/mL to about 2.5 μg/mL. In embodiments, the first target Cₘₐₓ is about 2.0 μg/mL to about 2.5 μg/mL. In embodiments, the first target Cₘₐₓ is about 0.5 μg/mL to about 2.0 μg/mL. In embodiments, the first target Cₘₐₓ is about 1.5 μg/mL to about 2.0 μg/mL. In embodiments, the first target Cₘₐₓ is about 0.5 μg/mL to about 1.5 μg/mL. In embodiments, the first target Cₘₐₓ is about 1.0 μg/mL to about 1.5 μg/mL. In embodiments, the first target Cₘₐₓ is about 0.5 μg/mL to about 1.0 μg/mL.

In embodiments, the first target Cₘᵢₙ is about 0.25 μg/mL to about 1.5 μg/mL. In embodiments, the first target Cₘᵢₙ is about 0.25 μg/mL to about 1 μg/mL. In embodiments, the first target Cₘᵢₙ is about 0.5 μg/mL to about 1.5 μg/mL. In embodiments, the first target Cₘᵢₙ is about 0.5 μg/mL to about 1.0 μg/mL. In embodiments, the first target Cₘᵢₙ is about 0.75 μg/mL to about 1.5 μg/mL. In embodiments, the first target Cₘᵢₙ is about 0.25 μg/mL to about 1.25 μg/mL. In embodiments, the first target Cₘᵢₙ is about 1.0 μg/mL to about 1.5 μg/mL. In embodiments, the first target Cₘᵢₙ is about 1.25 μg/mL to about 1.5 μg/mL. In embodiments, the first target Cₘᵢₙ is about 0.5 μg/mL to about 1.25 μg/mL. In embodiments, the first target Cₘᵢₙ is about 0.75 μg/mL to about 1.25 μg/mL. In embodiments, the first target Cₘᵢₙ is about 1.0 μg/mL to about 1.25 μg/mL. In embodiments, the first target Cₘᵢₙ is about 0.75 μg/mL to about 1 μg/mL. In embodiments, the first target Cₘᵢₙ is about 0.25 μg/mL to about 0.75 μg/mL. In embodiments, the first target Cₘᵢₙ is about 0.5 μg/mL to about 0.75 μg/mL. In embodiments, the first target Cₘᵢₙ is about 0.25 μg/mL to about 0.5 μg/mL.

In embodiments, the second target Cₘₐₓ is about 1.5 μg/mL to about 4.0 μg/mL. In embodiments, the second target Cₘₐₓ is about 1.5 μg/mL to about 3.0 μg/mL. In embodiments, the second target Cₘₐₓ is about 2.5 μg/mL to about 4.0 μg/mL. In embodiments, the second target Cₘₐₓ is about 2.0 μg/mL to about 4.0 μg/mL. In embodiments, the second target Cₘₐₓ is about 2.0 μg/mL to about 3.0 μg/mL. In embodiments, the second target Cₘₐₓ is about 3.0 μg/mL to about 4.0 μg/mL. In embodiments, the second target Cₘₐₓ is about 0.5 μg/mL to about 4.0 μg/mL. In embodiments, the second target Cₘₐₓ is about 1.0 μg/mL to about 4.0 μg/mL. In embodiments, the second target Cₘₐₓ is about 3.5 μg/mL to about 4.0 μg/mL. In embodiments, the second target Cₘₐₓ is about 0.5 μg/mL to about 3.5 μg/mL. In embodiments, the second target Cₘₐₓ is about 1.0 μg/mL to about 3.5 μg/mL. In embodiments, the second target Cₘₐₓ is about 1.5 μg/mL to about 3.5 μg/mL. In embodiments, the second target Cₘₐₓ is about 2.0 μg/mL to about 3.5 μg/mL. In embodiments, the second target Cₘₐₓ is about 2.5 μg/mL to about 3.5 μg/mL. In embodiments, the second target Cₘₐₓ is about 3.0 μg/mL to about 3.5 μg/mL. In embodiments, the second target Cₘₐₓ is about 0.5 μg/mL to about 3.0 μg/mL. In embodiments, the second target Cₘₐₓ is about 1.0 μg/mL to about 3.0 μg/mL. In embodiments, the second target Cₘₐₓ is about 2.5 μg/mL to about 3.0 μg/mL. In embodiments, the second target Cₘₐₓ is about 0.5 μg/mL to about 2.5 μg/mL. In embodiments, the second target Cₘₐₓ is about 1.0 μg/mL to about 2.5 μg/mL. In embodiments, the second target Cₘₐₓ is about 1.5 μg/mL to about 2.5 μg/mL. In embodiments, the second target Cₘₐₓ is about 2.0 μg/mL to about 2.5 μg/mL. In embodiments, the second target Cₘₐₓ is about 0.5 μg/mL to about 2.0 μg/mL. In embodiments, the second target Cₘₐₓ is about 1.0 μg/mL to about 2.0 μg/mL. In embodiments, the second target Cₘₐₓ is about 1.5 μg/mL to about 2.0 μg/mL. In embodiments, the second target Cₘₐₓ is about 0.5 μg/mL to about 1.5 μg/mL. In embodiments, the second target Cₘₐₓ is about 1.0 μg/mL to about 1.5 μg/mL. In embodiments, the second target Cₘₐₓ is about 0.5 μg/mL to about 1.0 μg/mL.

In embodiments, the second target Cₘᵢₙ is about 0.1 μg/mL to about 1.0 μg/mL. In embodiments, the second target Cₘᵢₙ is about 0.25 μg/mL to about 1.0 μg/mL. In embodiments, the second target Cₘᵢₙ is about 0.5 μg/mL to about 1.0 μg/mL. In embodiments, the second target Cₘᵢₙ is about 0.75 μg/mL to about 1.0 μg/mL. In embodiments, the second target Cₘᵢₙ is about 0.05 μg/mL to about 1.0 μg/mL. In embodiments, the second target Cₘᵢₙ is about 0.01 μg/mL to about 1.0 μg/mL. In embodiments, the second target Cₘᵢₙ is about 0.01 μg/mL to about 0.75 μg/mL. In embodiments, the second target Cₘᵢₙ is about 0.05 μg/mL to about 0.75 μg/mL. In embodiments, the second target Cₘᵢₙ is about 0.1 μg/mL to about 0.75 μg/mL. In embodiments, the second target Cₘᵢₙ is about 0.25 μg/mL to about 0.75 μg/mL. In embodiments, the second target Cₘᵢₙ is about 0.5 μg/mL to about 0.75 μg/mL. In embodiments, the second target Cₘᵢₙ is about 0.01 μg/mL to about 0.5 μg/mL. In embodiments, the second target Cₘᵢₙ is about 0.05 μg/mL to about 0.5 μg/mL. In embodiments, the second target Cₘᵢₙ is about 0.1 μg/mL to about 0.5 μg/mL. In embodiments, the second target Cₘᵢₙ is about 0.25 μg/mL to about 0.5 μg/mL. In embodiments, the second target Cₘᵢₙ is about 0.01 μg/mL to about 0.25 μg/mL. In embodiments, the second target Cₘᵢₙ is about 0.05 μg/mL to about 0.25 μg/mL. In embodiments, the second target Cₘᵢₙ is about 0.1 μg/mL to about 0.25 μg/mL. In embodiments, the second target Cₘᵢₙ is about 0.01 μg/mL to about 0.1 μg/mL. In embodiments, the second target Cₘᵢₙ is about 0.05 μg/mL to about 0.1 μg/mL. In embodiments, the second target Cₘᵢₙ is about 0.01 μg/mL to about 0.05 μg/mL.

In embodiments, the first target Cₘₐₓ is achieved at about 0.75 to about 4 hours (i.e., first Tₘₐₓ) after administration of the pharmaceutical composition. In embodiments, the first target Cₘₐₓ is achieved at about 1.5 to about 3 hours (i.e., first Tₘₐₓ) after administration of the pharmaceutical composition. In embodiments, the first target Cₘₐₓ is achieved at about 1.0 to about 4 hours (i.e., first Tₘₐₓ) after administration of the pharmaceutical composition. In embodiments, the first target Cₘₐₓ is achieved at about 1.5 to about 4 hours (i.e., first Tₘₐₓ) after administration of the pharmaceutical composition. In embodiments, the first target Cₘₐₓ is achieved at about 2.0 to about 4 hours (i.e., first Tₘₐₓ) after administration of the pharmaceutical composition. In embodiments, the first target Cₘₐₓ is achieved at about 2.5 to about 4 hours (i.e., first Tₘₐₓ) after administration of the pharmaceutical composition. In embodiments, the first target Cₘₐₓ is achieved at about 3.0 to about 4 hours (i.e., first Tₘₐₓ) after administration of the pharmaceutical composition. In embodiments, the first target Cₘₐₓ is achieved at about 3.5 to about 4 hours (i.e., first Tₘₐₓ) after administration of the pharmaceutical composition. In embodiments, the first target Cₘₐₓ is achieved at about 0.75 to about 3.5 hours (i.e., first Tₘₐₓ) after administration of the pharmaceutical composition. In embodiments, the first target Cₘₐₓ is achieved at about 1.0 to about 3.5 hours (i.e., first Tₘₐₓ) after administration of the pharmaceutical composition. In embodiments, the first target Cₘₐₓ is achieved at about 1.5 to about 3.5 hours (i.e., first Tₘₐₓ) after administration of the pharmaceutical composition. In embodiments, the first target Cₘₐₓ is achieved at about 2.0 to about 3.5 hours (i.e., first Tₘₐₓ) after administration of the pharmaceutical composition. In embodiments, the first target Cₘₐₓ is achieved at about 2.5 to about 3.5 hours (i.e., first Tₘₐₓ) after administration of the pharmaceutical composition. In embodiments, the first target Cₘₐₓ is achieved at about 3.0 to about 3.5 hours (i.e., first Tₘₐₓ) after administration of the pharmaceutical composition. In embodiments, the first target Cₘₐₓ is achieved at about 0.75 to about 3.0 hours (i.e., first Tₘₐₓ) after administration of the pharmaceutical composition. In embodiments, the first target Cₘₐₓ is achieved at about 1.0 to about 3.0 hours (i.e., first Tₘₐₓ) after administration of the pharmaceutical composition. In embodiments, the first target Cₘₐₓ is achieved at about 2.0 to about 3.0 hours (i.e., first Tₘₐₓ) after administration of the pharmaceutical composition. In embodiments, the first target Cₘₐₓ is achieved at about 2.5 to about 3.0 hours (i.e., first Tₘₐₓ) after administration of the pharmaceutical composition. In embodiments, the first target Cₘₐₓ is achieved at about 0.75 to about 2.5 hours (i.e., first Tₘₐₓ) after administration of the pharmaceutical composition. In embodiments, the first target Cₘₐₓ is achieved at about 1.0 to about 2.5 hours (i.e., first Tₘₐₓ) after administration of the pharmaceutical composition. In embodiments, the first target Cₘₐₓ is achieved at about 1.5 to about 2.5 hours (i.e., first Tₘₐₓ) after administration of the pharmaceutical composition. In embodiments, the first target Cₘₐₓ is achieved at about 2.0 to about 2.5 hours (i.e., first Tₘₐₓ) after administration of the pharmaceutical composition. In embodiments, the first target Cₘₐₓ is achieved at about 0.75 to about 2.0 hours (i.e., first Tₘₐₓ) after administration of the pharmaceutical composition. In embodiments, the first target Cₘₐₓ is achieved at about 1.0 to about 2.0 hours (i.e., first Tₘₐₓ) after administration of the pharmaceutical composition. In embodiments, the first target Cₘₐₓ is achieved at about 1.5 to about 2.0 hours (i.e., first Tₘₐₓ) after administration of the pharmaceutical composition. In embodiments, the first target Cₘₐₓ is achieved at about 0.75 to about 1.5 hours (i.e., first Tₘₐₓ) after administration of the pharmaceutical composition. In embodiments, the first target Cₘₐₓ is achieved at about 1.0 to about 1.5 hours (i.e., first Tₘₐₓ) after administration of the pharmaceutical composition. In embodiments, the first target Cₘₐₓ is achieved at about 0.75 to about 1.0 hours (i.e., first Tₘₐₓ) after administration of the pharmaceutical composition.

In embodiments, the first target Cₘᵢₙ is achieved at about 4 to about 8 hours (i.e., first Tₘᵢₙ) after administration of the pharmaceutical composition. In embodiments, the first target Cₘᵢₙ is achieved at about 5 to about 6 hours (i.e., first Tₘᵢₙ) after administration of the pharmaceutical composition. In embodiments, the first target Cₘᵢₙ is achieved at about 4.5 to about 8 hours (i.e., first Tₘᵢₙ) after administration of the pharmaceutical composition. In embodiments, the first target Cₘᵢₙ is achieved at about 5 to about 8 hours (i.e., first Tₘᵢₙ) after administration of the pharmaceutical composition. In embodiments, the first target Cₘᵢₙ is achieved at about 5.5 to about 8 hours (i.e., first Tₘᵢₙ) after administration of the pharmaceutical composition. In embodiments, the first target Cₘᵢₙ is achieved at about 6 to about 8 hours (i.e., first Tₘᵢₙ) after administration of the pharmaceutical composition. In embodiments, the first target Cₘᵢₙ is achieved at about 6.5 to about 8 hours (i.e., first Tₘᵢₙ) after administration of the pharmaceutical composition. In embodiments, the first target Cₘᵢₙ is achieved at about 7.0 to about 8 hours (i.e., first Tₘᵢₙ) after administration of the pharmaceutical composition. In embodiments, the first target Cₘᵢₙ is achieved at about 7.5 to about 8 hours (i.e., first Tₘᵢₙ) after administration of the pharmaceutical composition. In embodiments, the first target Cₘᵢₙ is achieved at about 4 to about 7.5 hours (i.e., first Tₘᵢₙ) after administration of the pharmaceutical composition. In embodiments, the first target Cₘᵢₙ is achieved at about 4.5 to about 7.5 hours (i.e., first Tₘᵢₙ) after administration of the pharmaceutical composition. In embodiments, the first target Cₘᵢₙ is achieved at about 5 to about 7.5 hours (i.e., first Tₘᵢₙ) after administration of the pharmaceutical composition. In embodiments, the first target Cₘᵢₙ is achieved at about 5.5 to about 7.5 hours (i.e., first Tₘᵢₙ) after administration of the pharmaceutical composition. In embodiments, the first target Cₘᵢₙ is achieved at about 6 to about 7.5 hours (i.e., first Tₘᵢₙ) after administration of the pharmaceutical composition. In embodiments, the first target Cₘᵢₙ is achieved at about 6.5 to about 7.5 hours (i.e., first Tₘᵢₙ) after administration of the pharmaceutical composition. In embodiments, the first target Cₘᵢₙ is achieved at about 7.0 to about 7.5 hours (i.e., first Tₘᵢₙ) after administration of the pharmaceutical composition. In embodiments, the first target Cₘᵢₙ is achieved at about 4 to about 7.0 hours (i.e., first Tₘᵢₙ) after administration of the pharmaceutical composition. In embodiments, the first target Cₘᵢₙ is achieved at about 4.5 to about 7.0 hours (i.e., first Tₘᵢₙ) after administration of the pharmaceutical composition. In embodiments, the first target Cₘᵢₙ is achieved at about 5 to about 7.0 hours (i.e., first Tₘᵢₙ) after administration of the pharmaceutical composition. In embodiments, the first target Cₘᵢₙ is achieved at about 5.5 to about 7.0 hours (i.e., first Tₘᵢₙ) after administration of the pharmaceutical composition. In embodiments, the first target Cₘᵢₙ is achieved at about 6 to about 7.0 hours (i.e., first Tₘᵢₙ) after administration of the pharmaceutical composition. In embodiments, the first target Cₘᵢₙ is achieved at about 6.5 to about 7.0 hours (i.e., first Tₘᵢₙ) after administration of the pharmaceutical composition. In embodiments, the first target Cₘᵢₙ is achieved at about 4 to about 6.5 hours (i.e., first Tₘᵢₙ) after administration of the pharmaceutical composition. In embodiments, the first target Cₘᵢₙ is achieved at about 4.5 to about 6.5 hours (i.e., first Tₘᵢₙ) after administration of the pharmaceutical composition. In embodiments, the first target Cₘᵢₙ is achieved at about 5 to about 6.5 hours (i.e., first Tₘᵢₙ) after administration of the pharmaceutical composition. In embodiments, the first target Cₘᵢₙ is achieved at about 5.5 to about 6.5 hours (i.e., first Tₘᵢₙ) after administration of the pharmaceutical composition. In embodiments, the first target Cₘᵢₙ is achieved at about 6 to about 6.5 hours (i.e., first Tₘᵢₙ) after administration of the pharmaceutical composition. In embodiments, the first target Cₘᵢₙ is achieved at about 4 to about 6.0 hours (i.e., first Tₘᵢₙ) after administration of the pharmaceutical composition. In embodiments, the first target Cₘᵢₙ is achieved at about 4.5 to about 6.0 hours (i.e., first Tₘᵢₙ) after administration of the pharmaceutical composition. In embodiments, the first target Cₘᵢₙ is achieved at about 5.5 to about 6.0 hours (i.e., first Tₘᵢₙ) after administration of the pharmaceutical composition. In embodiments, the first target Cₘᵢₙ is achieved at about 4 to about 5.5 hours (i.e., first Tₘᵢₙ) after administration of the pharmaceutical composition. In embodiments, the first target Cₘᵢₙ is achieved at about 4.5 to about 5.5 hours (i.e., first Tₘᵢₙ) after administration of the pharmaceutical composition. In embodiments, the first target Cₘᵢₙ is achieved at about 5 to about 5.5 hours (i.e., first Tₘᵢₙ) after administration of the pharmaceutical composition. In embodiments, the first target Cₘᵢₙ is achieved at about 4 to about 5.0 hours (i.e., first Tₘᵢₙ) after administration of the pharmaceutical composition. In embodiments, the first target Cₘᵢₙ is achieved at about 4.5 to about 5.0 hours (i.e., first Tₘᵢₙ) after administration of the pharmaceutical composition. In embodiments, the first target Cₘᵢₙ is achieved at about 4 to about 4.5 hours (i.e., first Tₘᵢₙ) after administration of the pharmaceutical composition.

In embodiments, the second target Cₘₐₓ is achieved at about 12 to about 20 hours (i.e., second Tₘₐₓ) after administration of the pharmaceutical composition. In embodiments, the second target Cₘₐₓ is achieved at about 14 to about 16 hours (i.e., second Tₘₐₓ) after administration of the pharmaceutical composition. In embodiments, the second target Cₘₐₓ is achieved at about 2.75 to about 16 hours (i.e., second Tₘₐₓ) after administration of the pharmaceutical composition. In embodiments, the second target Cₘₐₓ is achieved at about 4 to about 16 hours (i.e., second Tₘₐₓ) after administration of the pharmaceutical composition. In embodiments, the second target Cₘₐₓ is achieved at about 6 to about 16 hours (i.e., second Tₘₐₓ) after administration of the pharmaceutical composition. In embodiments, the second target Cₘₐₓ is achieved at about 8 to about 16 hours (i.e., second Tₘₐₓ) after administration of the pharmaceutical composition. In embodiments, the second target Cₘₐₓ is achieved at about 10 to about 16 hours (i.e., second Tₘₐₓ) after administration of the pharmaceutical composition. In embodiments, the second target Cₘₐₓ is achieved at about 12 to about 16 hours (i.e., second Tₘₐₓ) after administration of the pharmaceutical composition. In embodiments, the second target Cₘₐₓ is achieved at about 2.75 to about 14 hours (i.e., second Tₘₐₓ) after administration of the pharmaceutical composition. In embodiments, the second target Cₘₐₓ is achieved at about 4 to about 14 hours (i.e., second Tₘₐₓ) after administration of the pharmaceutical composition. In embodiments, the second target Cₘₐₓ is achieved at about 6 to about 14 hours (i.e., second Tₘₐₓ) after administration of the pharmaceutical composition. In embodiments, the second target Cₘₐₓ is achieved at about 8 to about 14 hours (i.e., second Tₘₐₓ) after administration of the pharmaceutical composition. In embodiments, the second target Cₘₐₓ is achieved at about 10 to about 14 hours (i.e., second Tₘₐₓ) after administration of the pharmaceutical composition. In embodiments, the second target Cₘₐₓ is achieved at about 12 to about 14 hours (i.e., second Tₘₐₓ) after administration of the pharmaceutical composition. In embodiments, the second target Cₘₐₓ is achieved at about 2.75 to about 12 hours (i.e., second Tₘₐₓ) after administration of the pharmaceutical composition. In embodiments, the second target Cₘₐₓ is achieved at about 4 to about 12 hours (i.e., second Tₘₐₓ) after administration of the pharmaceutical composition. In embodiments, the second target Cₘₐₓ is achieved at about 6 to about 12 hours (i.e., second Tₘₐₓ) after administration of the pharmaceutical composition. In embodiments, the second target Cₘₐₓ is achieved at about 8 to about 12 hours (i.e., second Tₘₐₓ) after administration of the pharmaceutical composition. In embodiments, the second target Cₘₐₓ is achieved at about 10 to about 12 hours (i.e., second Tₘₐₓ) after administration of the pharmaceutical composition. In embodiments, the second target Cₘₐₓ is achieved at about 2.75 to about 10 hours (i.e., second Tₘₐₓ) after administration of the pharmaceutical composition. In embodiments, the second target Cₘₐₓ is achieved at about 4 to about 10 hours (i.e., second Tₘₐₓ) after administration of the pharmaceutical composition. In embodiments, the second target Cₘₐₓ is achieved at about 6 to about 10 hours (i.e., second Tₘₐₓ) after administration of the pharmaceutical composition. In embodiments, the second target Cₘₐₓ is achieved at about 8 to about 10 hours (i.e., second Tₘₐₓ) after administration of the pharmaceutical composition. In embodiments, the second target Cₘₐₓ is achieved at about 2.75 to about 8 hours (i.e., second Tₘₐₓ) after administration of the pharmaceutical composition. In embodiments, the second target Cₘₐₓ is achieved at about 4 to about 8 hours (i.e., second Tₘₐₓ) after administration of the pharmaceutical composition. In embodiments, the second target Cₘₐₓ is achieved at about 6 to about 8 hours (i.e., second Tₘₐₓ) after administration of the pharmaceutical composition. In embodiments, the second target Cₘₐₓ is achieved at about 2.75 to about 6 hours (i.e., second Tₘₐₓ) after administration of the pharmaceutical composition. In embodiments, the second target Cₘₐₓ is achieved at about 4 to about 6 hours (i.e., second Tₘₐₓ) after administration of the pharmaceutical composition. In embodiments, the second target Cₘₐₓ is achieved at about 2.75 to about 4 hours (i.e., second Tₘₐₓ) after administration of the pharmaceutical composition. In embodiments, the second target Cₘₐₓ is achieved at about 16, about 17, about 18, about 19 or about 20 hours.

In embodiments, the second target Cₘₐₓ is achieved at about 2 to about 8 hours (i.e., second Tₘₐₓ) after the first target Cₘᵢₙ. In embodiments, the second target Cₘₐₓ is achieved at about 4 to about 6 hours (i.e., second Tₘₐₓ) after the first target Cₘᵢₙ. In embodiments, the second target Cₘₐₓ is achieved at about 3 to about 8 hours (i.e., second Tₘₐₓ) after the first target Cₘₐₓ. In embodiments, the second target Cₘₐₓ is achieved at about 4 to about 8 hours (i.e., second Tₘₐₓ) after the first target Cₘᵢₙ. In embodiments, the second target Cₘₐₓ is achieved at about 5 to about 8 hours (i.e., second Tₘₐₓ) after the target first Cₘᵢₙ. In embodiments, the second target Cₘₐₓ is achieved at about 6 to about 8 hours (i.e., second Tₘₐₓ) after the first target Cₘᵢₙ. In embodiments, the second target Cₘₐₓ is achieved at about 7 to about 8 hours (i.e., second Tₘₐₓ) after the first target Cₘᵢₙ. In embodiments, the second target Cₘₐₓ is achieved at about 2 to about 7 hours (i.e., second Tₘₐₓ) after the first target Cₘᵢₙ. In embodiments, the second target Cₘₐₓ is achieved at about 3 to about 7 hours (i.e., second Tₘₐₓ) after the first target Cₘᵢₙ. In embodiments, the second target Cₘₐₓ is achieved at about 4 to about 7 hours (i.e., second Tₘₐₓ) after the first target Cₘᵢₙ. In embodiments, the second target Cₘₐₓ is achieved at about 5 to about 7 hours (i.e., second Tₘₐₓ) after the first target Cₘᵢₙ. In embodiments, the second target Cₘₐₓ is achieved at about 6 to about 7 hours (i.e., second Tₘₐₓ) after the first target Cₘᵢₙ. In embodiments, the second target Cₘₐₓ is achieved at about 2 to about 6 hours (i.e., second Tₘₐₓ) after the first target Cₘᵢₙ. In embodiments, the second target Cₘₐₓ is achieved at about 3 to about 6 hours (i.e., second Tₘₐₓ) after the first target Cₘᵢₙ. In embodiments, the second target Cₘₐₓ is achieved at about 5 to about 6 hours (i.e., second Tₘₐₓ) after the first target Cₘᵢₙ. In embodiments, the second target Cₘₐₓ is achieved at about 2 to about 5 hours (i.e., second Tₘₐₓ) after the first target Cₘᵢₙ. In embodiments, the second target Cₘₐₓ is achieved at about 3 to about 5 hours (i.e., second Tₘₐₓ) after the first target Cₘᵢₙ. In embodiments, the second target Cₘₐₓ is achieved at about 4 to about 5 hours (i.e., second Tₘₐₓ) after the first target Cₘᵢₙ. In embodiments, the second target Cₘₐₓ is achieved at about 2 to about 4 hours (i.e., second Tₘₐₓ) after the first target Cₘᵢₙ. In embodiments, the second target Cₘₐₓ is achieved at about 3 to about 4 hours (i.e., second Tₘₐₓ) after the first target Cₘᵢₙ. In embodiments, the second target Cₘₐₓ is achieved at about 2 to about 3 hours (i.e., second Tₘₐₓ) after the first target Cₘᵢₙ.

In embodiments, the time between the first target Cₘₐₓ and the second target Cₘₐₓ is about 2 to about 8 hours. In embodiments, the time between the first target Cₘₐₓand the second target Cₘₐₓ is about 3 to about 7 hours. In embodiments, the time between the first target Cₘₐₓ and the second target Cₘₐₓ is about 4 to about 6 hours. In embodiments, the time between the first target Cₘₐₓ and the second target Cₘₐₓ is about 3 to about 8 hours. In embodiments, the time between the first target Cₘₐₓ and the second target Cₘₐₓ is about 4 to about 8 hours. In embodiments, the time between the first target Cₘₐₓ and the second target Cₘₐₓ is about 5 to about 8 hours. In embodiments, the time between the first target Cₘₐₓ and the second target Cₘₐₓ is about 6 to about 8 hours. In embodiments, the time between the first target Cₘₐₓ and the second target Cₘₐₓ is about 7 to about 8 hours. In embodiments, the time between the first target Cₘₐₓ and the second target Cₘₐₓ is about 2 to about 7 hours. In embodiments, the time between the first target Cₘₐₓ and the second target Cₘₐₓ is about 4 to about 7 hours. In embodiments, the time between the first target Cₘₐₓ and the second target Cₘₐₓ is about 5 to about 7 hours. In embodiments, the time between the first target Cₘₐₓ and the second target Cₘₐₓ is about 6 to about 7 hours. In embodiments, the time between the first target Cₘₐₓ and the second target Cₘₐₓ is about 2 to about 6 hours. In embodiments, the time between the first target Cₘₐₓ and the second target Cₘₐₓ is about 3 to about 6 hours. In embodiments, the time between the first target Cₘₐₓ and the second target Cₘₐₓ is about 5 to about 6 hours. In embodiments, the time between the first target Cₘₐₓ and the second target Cₘₐₓ is about 2 to about 5 hours. In embodiments, the time between the first target Cₘₐₓ and the second target Cₘₐₓ is about 3 to about 5 hours. In embodiments, the time between the first target Cₘₐₓ and the second target Cₘₐₓ is about 4 to about 5 hours. In embodiments, the time between the first target Cₘₐₓ and the second target Cₘₐₓ is about 2 to about 4 hours. In embodiments, the time between the first target Cₘₐₓ and the second target Cₘₐₓ is about 3 to about 4 hours. In embodiments, the time between the first target Cₘₐₓ and the second target Cₘₐₓ is about 2 to about 3 hours.

In embodiments, the second target Cₘᵢₙ is achieved before about 24 hours (i.e., second Tₘᵢₙ) after administration of the pharmaceutical composition. In embodiments, the second target Cₘᵢₙ is achieved before about 20 hours (i.e., second Tₘᵢₙ) after administration of the pharmaceutical composition. In embodiments, the second target Cₘᵢₙ is achieved before about 16 hours (i.e., second Tₘᵢₙ) after administration of the pharmaceutical composition. In embodiments the second target Cₘᵢₙ is achieved at about 24 hours. In embodiments the second target Cₘᵢₙ is achieved at about 23 hours. In embodiments the second target Cₘᵢₙ is achieved at about 22 hours. In embodiments the second target Cₘᵢₙ is achieved at about 21 hours. In embodiments the second target Cₘₐₓ is achieved at about 20 hours. In embodiments the second target Cₘᵢₙ is achieved at about 19 hours. In embodiments the second target Cₘᵢₙ is achieved at about 18 hours. In embodiments the second target Cₘᵢₙ is achieved at about 17 hours. In embodiments the second target Cₘᵢₙ is achieved at about 16 hours.

In embodiments, the first target Cₘₐₓ may be achieved through a first release of solabegron and the second target Cₘₐₓ may be achieved through a second release of solabegron. In embodiments, the first target Cₘₐₓ may be achieved during or after a first release of solabegron; that is the first target Cₘₐₓ may be achieved after the start of the first release. In embodiments, the second target Cₘₐₓ may be achieved during or after a second release of solabegron; that is the second target Cₘₐₓ may be achieved after the start of the second release. In embodiments, the first target Cₘᵢₙ may be achieved after the first target Cₘₐₓ and before the second target Cₘₐₓ. In embodiments, the second target Cₘᵢₙ may be achieved after the second target Cₘₐₓ. In embodiments, the pharmaceutical composition is a single unit dose. In embodiments, the pharmaceutical composition is two unit doses.

In one embodiment, the present application describes a pharmaceutical composition comprising a therapeutically effective amount of the amorphous solid form of solabegron, wherein the pharmaceutical composition achieves a first target Cₘₐₓ, a second target Cₘₐₓ, a first target Cₘᵢₙ between the first target Cₘₐₓ and the second target Cₘₐₓ, and a second target Cₘᵢₙ after the second target Cₘₐₓ, wherein the first target Cₘₐₓ, the second target Cₘₐₓ, the first target Cₘᵢₙ, and second target Cₘᵢₙ are as described previously. In one embodiment, the present application describes a pharmaceutical composition comprising a therapeutically effective amount of the hydrochloride salt form of solabegron, wherein the pharmaceutical composition achieves a first target Cₘₐₓ, a second target Cₘₐₓ, a first target Cₘᵢₙ between the first target Cₘₐₓ and the second target Cₘₐₓ, and a second target Cₘᵢₙ after the second target Cₘₐₓ, wherein the first target Cₘₐₓ, the second target Cₘₐₓ, the first target Cₘᵢₙ, and second target Cₘᵢₙ are as described previously. In one embodiment, the present application describes a pharmaceutical composition comprising a therapeutically effective amount of the zwitterion form of solabegron, wherein the pharmaceutical composition achieves a first target Cₘₐₓ, a second target Cₘₐₓ, a first target Cₘᵢₙ between the first target Cₘₐₓ and the second target Cₘₐₓ, and a second target Cₘᵢₙ after the second target Cₘₐₓ, wherein the first target Cₘₐₓ, the second target Cₘₐₓ, the first target Cₘᵢₙ, and second target Cₘᵢₙ are as described previously. Further embodiments describe pharmaceutical compositions, wherein said pharmaceutical composition achieves a plasma concentration of about 1 μg/mL or less for about 6 hours to about 9 hours during a twenty-four hour period. Further embodiments describe pharmaceutical compositions, wherein said pharmaceutical composition achieves a target AUC of about 5,000 ng hr/ml to about 30,000 ng hr/mL. Further embodiments describe pharmaceutical compositions, further comprising two separate and distinct releases of solabegron. Further embodiments describe pharmaceutical compositions, wherein the two releases are contained within two separate and distinct drug delivery systems. Further embodiments describe pharmaceutical, wherein the two separate and distinct drug delivery systems are administered BID. Further embodiments describe pharmaceutical compositions, wherein the BID administration is separated by a period of between about 6 to about 18 hours. Further embodiments describe pharmaceutical compositions, wherein the two releases are contained within the same drug delivery system. Further embodiments describe pharmaceutical compositions, wherein the delivery vehicle is selected from the group consisting of: tablets; bi-layer tablets; multilayer tablets, capsules; spray dry formulations, multi particulates; drug coated spheres/pellets; matrix tablets; and multi core tablets. Further embodiments describe pharmaceutical compositions, wherein the first target Cₘₐₓ is achieved after the start of a first release of solabegron and the second target Cₘₐₓ is achieved after the start of a second release of solabegron. Further embodiments describe pharmaceutical compositions, wherein said first target Cₘₐₓ is about 0.5 μg/mL to about 3.5 μg/mL. Further embodiments describe pharmaceutical compositions, wherein said second target Cₘₐₓ is about 1.5 μg.mL to about 4 μg/mL. Further embodiments describe pharmaceutical compositions, wherein said first target Cₘᵢₙ is about 0.25 μg/mL to about 1.5 μg/mL. Further embodiments describe pharmaceutical compositions, wherein said second target Cₘᵢₙ is about 0.01 μg/mL to about 1.0 μg/mL. Further embodiments describe pharmaceutical compositions, wherein the time between the first target Cₘₐₓ and the second target Cₘₐₓ is about 2 to about 8 hours. Further embodiments describe pharmaceutical compositions, wherein the first target Cₘᵢₙ is achieved at about 4 to about 8 hours after the first administration. Further embodiments describe pharmaceutical compositions, wherein the second target Cₘᵢₙ is achieved before about 24 hours after administration of the pharmaceutical composition. Further embodiments describe pharmaceutical compositions, wherein the first target Cₘₐₓ is achieved at about 0.75 to about 4 hours after the first administration. Further embodiments describe pharmaceutical compositions, wherein the second target Cₘₐₓ is achieved at about 2 to about 8 hours after the first target Cₘᵢₙ. Further embodiments describe pharmaceutical compositions, wherein the first release comprises about 30 mg to about 500 mg of solabegron. Further embodiments describe pharmaceutical compositions, wherein the second release comprises about 30 mg to about 500 mg of solabegron. Further embodiments describe pharmaceutical compositions, further comprising one or more additional therapeutic agents selected from the group consisting of: antimuscarinic agents; alpha adrenoceptor blockers; botulinum toxin; purinergics; cannabinoids; transient receptor potential (TRP) protein inhibitors; prostaglandins; percutaneous tibial nerve stimulation; 5-alpha reductase inhibitors; and phosphodiesterase-5 inhibitors.

In embodiments, the pharmaceutical composition achieves a target area under the curve (herein after AUC) of about 5,000 ng.hr/mL to about 30,000 ng.hr/mL over a 24 hour period. In embodiments, the pharmaceutical composition achieves a target area under the curve of about 10,000 ng.hr/mL to about 30,000 ng.hr/mL over a 24 hour period. In embodiments, the pharmaceutical composition achieves a target area under the curve of about 15,000 ng.hr/mL to about 30,000 ng.hr/mL over a 24 hour period. In embodiments, the pharmaceutical composition achieves a target area under the curve of about 20,000 ng.hr/mL to about 30,000 ng.hr/mL over a 24 hour period. In embodiments, the pharmaceutical composition achieves a target area under the curve of about 25,000 ng.hr/mL to about 30,000 ng.hr/mL over a 24 hour period. In embodiments, the pharmaceutical composition achieves a target area under the curve of about 5,000 ng.hr/mL to about 25,000 ng.hr/mL over a 24 hour period. In embodiments, the pharmaceutical composition achieves a target area under the curve of about 10,000 ng.hr/mL to about 25,000 ng.hr/mL over a 24 hour period. In embodiments, the pharmaceutical composition achieves a target area under the curve of about 15,000 ng.hr/mL to about 25,000 ng.hr/mL over a 24 hour period. In embodiments, the pharmaceutical composition achieves a target area under the curve of about 20,000 ng.hr/mL to about 25,000 ng.hr/mL over a 24 hour period. In embodiments, the pharmaceutical composition achieves a target area under the curve of about 5,000 ng.hr/mL to about 20,000 ng.hr/mL over a 24 hour period. In embodiments, the pharmaceutical composition achieves a target area under the curve of about 10,000 ng.hr/mL to about 20,000 ng.hr/mL over a 24 hour period. In embodiments, the pharmaceutical composition achieves a target area under the curve of about 15,000 ng.hr/mL to about 20,000 ng.hr/mL over a 24 hour period. In embodiments, the pharmaceutical composition achieves a target area under the curve of about 5,000 ng.hr/mL to about 15,000 ng.hr/mL over a 24 hour period. In embodiments, the pharmaceutical composition achieves a target area under the curve of about 10,000 ng.hr/mL to about 15,000 ng.hr/mL over a 24 hour period. In embodiments, the pharmaceutical composition achieves a target area under the curve of about 5,000 ng.hr/mL to about 10,000 ng.hr/mL over a 24 hour period. In embodiments, the pharmaceutical composition achieves a target area under the curve of about 5,000 ng.hr/mL over a 24 hour period; about 6,000 ng.hr/mL over a 24 hour period; about 7,000 ng.hr/mL over a 24 hour period; about 8,000 ng.hr/mL over a 24 hour period; about 9,000 ng.hr/mL over a 24 hour period; about 10,000 ng.hr/mL over a 24 hour period; about 11,000 ng.hr/mL over a 24 hour period; about 12,000 ng.hr/mL over a 24 hour period; about 13,000 ng.hr/mL over a 24 hour period; about 14,000 ng.hr/mL over a 24 hour period; about 15,000 ng.hr/mL over a 24 hour period; about 16,000 ng.hr/mL over a 24 hour period; about 17,000 ng.hr/mL over a 24 hour period; about 18,000 ng.hr/mL over a 24 hour period; about 19,000 ng.hr/mL over a 24 hour period; about 20,000 ng.hr/mL over a 24 hour period; about 21,000 ng.hr/mL over a 24 hour period; about 22,000 ng.hr/mL over a 24 hour period; about 23,000 ng.hr/mL over a 24 hour period; about 24,000 ng.hr/mL over a 24 hour period; about 25,000 ng.hr/mL over a 24 hour period; about 26,000 ng.hr/mL over a 24 hour period; about 27,000 ng.hr/mL over a 24 hour period; about 28,000 ng.hr/mL over a 24 hour period; about 29,000 ng.hr/mL over a 24 hour period; or about 30,000 ng.hr/mL over a 24 hour period.

In embodiments, the plasma concentration [C] of solabegron is about 1 μg/mL or below for a period of time of about 6 hours to about 9 hours during a 24 hour period. In embodiments, the plasma concentration [C] of solabegron is about 1 μg/mL or below for a period of time of about 7 hours to about 8 hours during a 24 hour period. In embodiments, the plasma concentration [C] of solabegron is about 1 μg/mL or below for a period of time of about 7 hours to about 9 hours during a 24 hour period. In embodiments, the plasma concentration [C] of solabegron is about 1 μg/mL or below for a period of time of about 8 hours to about 9 hours during a 24 hour period. In embodiments, the plasma concentration [C] of solabegron is about 1 μg/mL or below for a period of time of about 6 hours to about 8 hours during a 24 hour period. In embodiments, the plasma concentration [C] of solabegron is about 1 μg/mL or below for a period of time of about 6 hours to about 7 hours during a 24 hour period.

In embodiments, the pharmaceutical composition provides a therapeutic benefit for about 15 to about 22 hours during a 24 hour period. In embodiments, the pharmaceutical composition provides a therapeutic benefit for about 15 hours during a 24 hour period. In embodiments, the pharmaceutical composition provides a therapeutic benefit for about 16 hours during a 24 hour period. In embodiments, the pharmaceutical composition provides a therapeutic benefit for about 17 hours during a 24 hour period. In embodiments, the pharmaceutical composition provides a therapeutic benefit for about 18 hours during a 24 hour period. In embodiments, the pharmaceutical composition provides a therapeutic benefit for about 19 hours during a 24 hour period. In embodiments, the pharmaceutical composition provides a therapeutic benefit for about 20 hours during a 24 hour period. In embodiments, the pharmaceutical composition provides a therapeutic benefit for about 21 hours during a 24 hour period. In embodiments, the pharmaceutical composition provides a therapeutic benefit for about 22 hours during a 24 hour period.

In embodiments, the pharmaceutical composition provides a therapeutically effective [C] for about 15 hours to about 22 hours during a 24 period. In embodiments, the pharmaceutical composition provides a therapeutically effective [C] for about 15 hours during a 24 period. In embodiments, the pharmaceutical composition provides a therapeutically effective [C] for about 16 hours during a 24 period. In embodiments, the pharmaceutical composition provides a therapeutically effective [C] for about 17 hours during a 24 period. In embodiments, the pharmaceutical composition provides a therapeutically effective [C] for about 18 hours during a 24 period. In embodiments, the pharmaceutical composition provides a therapeutically effective [C] for about 19 hours during a 24 period. In embodiments, the pharmaceutical composition provides a therapeutically effective [C] for about 20 hours during a 24 period. In embodiments, the pharmaceutical composition provides a therapeutically effective [C] for about 21 hours during a 24 period. In embodiments, the pharmaceutical composition provides a therapeutically effective [C] for about 22 hours during a 24 period.

Further embodiments describe pharmaceutical compositions further comprising two separate and distinct releases of solabegron. Further embodiments describe pharmaceutical compositions, wherein the first target Cₘₐₓ is achieved after the start of a first release of solabegron and the second target Cₘₐₓ is achieved after the start of a second release of solabegron.

In embodiments, the first release of solabegron may be a pulsatile release of solabegron. In embodiments, the second release of solabegron may be a pulsatile release of solabegron. In embodiments, the first release of solabegron may be an immediate release of solabegron. In embodiments, the second release of solabegron may be an immediate release of solabegron. In embodiments, the first release of solabegron may be modified release of solabegron. In embodiments, the second release of solabegron may be a modified release of solabegron. In embodiments, the first release of solabegron may be an extended release of solabegron. In embodiments, the second release of solabegron may be an extended release of solabegron. In embodiments, the first release of solabegron may be a delayed release of solabegron. In embodiments, the second release of solabegron may be a delayed release of solabegron. In embodiments, the first release of solabegron may be a multiparticulate formulation of solabegron. In embodiments, the second release of solabegron may be a multiparticulate formulation of solabegron. In embodiments, the first release of solabegron may be a matrix formulation of solabegron. In embodiments, the second release of solabegron may be a matrix formulation of solabegron. In embodiments, the first and second release of solabegron may be any combination of the foregoing. In embodiments the first release of solabegron and the second release of solabegron may be in a single pharmaceutical composition or in separate pharmaceutical compositions.

In embodiments, the first release of solabegron and the second release of solabegron may be identical amounts or may be different amounts of solabegron. In embodiments, the first release of solabegron may be about 30 mg to about 500 mg. In embodiments, the first release of solabegron may be about 75 mg to about 400 mg. In embodiments, the first release of solabegron may be about 75 mg to about 250 mg. In embodiments, the second release of solabegron may be about 30 mg to about 500 mg. In embodiments, the second release of solabegron may be about 100 mg to about 400 mg. In embodiments, the first release and the second release of solabegron may be about 125 mg. In embodiments, the first release and the second release of solabegron may be about 200 mg. In embodiments, the first release of solabegron may be about 125 mg and the second release of solabegron may be about 200 mg. In embodiments the first release of solabegron may be about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg or about 500 mg. In embodiments the second release of solabegron may be 30 mg, about 35 mg, about 40 mg, about 45 mg, about 50 mg, about 55 mg, about 60 mg, about 65 mg, about 75 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg or about 500 mg. In embodiments the first release of solabegron and the second release of solabegron may be in a single pharmaceutical composition or in separate pharmaceutical compositions.

In embodiments, the pharmaceutical compositions, further comprises one or more additional therapeutic agents selected from the group consisting of: antimuscarinic agents; alpha adrenoceptor blockers; botulinum toxin; purinergics; cannabinoids; transient receptor potential (TRP) protein inhibitors; prostaglandins; percutaneous tibial nerve stimulation; 5-alpha reductase inhibitors; and phosphodiesterase-5 inhibitors. In embodiments, the antimuscarinic agent may be tolterodine, oxybutynin, trospium, solifenacin, darifenacin, propiverine, fesoterodine, and pharmaceutically acceptable salts thereof. In embodiments, alpha adrenoceptor blockers may be tamuslosin, alfuzosin, and silodosin and pharmaceutically acceptable salts thereof. In embodiments, 5-alpha reductase inhibitors may be finasteride, dutaseteride and pharmaceutically acceptable salts thereof. In embodiments, phosphodiesterase-5 inhibitors may be sildenafil, tadaiafil, vardenafil, udenafil, avanafil and pharmaceutically acceptable salts thereof

In embodiments, the present application describes a pharmaceutical composition comprising a multiparticulate formulation of mini-tablets each comprising a therapeutically effective amount of solabegron.In embodiments, the multiparticulate formulation may comprise at least two populations of pellets containing solabegron. In embodiments, a first population of pellets is immediate release and a second population is delayed, sustained or modified release. In embodiments, the first population of pellets release the solabegron immediately in the upper GI tract and the second population of pellets release the solabegron later in a lower portion of the GI tract. In embodiments, the second population of pellets that are delayed, sustained or modified release may be coated with a pH dependent coating or a time dependent coating so as to delay the second release of solabegron to the desired position in the GI tract. In embodiments, the pellets may be drug-layered and/or matrix-type pellets.

In embodiments, the pharmaceutical composition may be a drug-coated sphere(s) formulation. In embodiments, the formulation may comprise at least two populations of drug-coated spheres containing solabegron. In embodiments, a first population of drug-coated spheres release the solabegron immediately in the upper GI tract and the second population of drug-coated spheres release the solabegron later in a lower portion of the GI tract. In embodiments, the second population of drug-coated spheres may be coated with a pH dependent coating or a time dependent coating so as to delay the second release of solabegron to the desired position in the GI tract. In embodiments, the pharmaceutical composition is a single unit dose. In embodiments, the pharmaceutical composition is two unit doses. In further embodiments the spheres allow for a drug load of solabegron of greater than about 30 weight percent of the composition; greater than about 35 weight percent of the composition; greater than about 40 weight percent of the composition, greater than about 50 weight percent of the composition, greater than about 55 weight percent of the composition or greater than about 60 weight percent of the composition.

In embodiments, the pharmaceutical composition may be a bi-layer tablet or a dual-encapsulated capsule. In embodiments, the bi-layer tablet may comprise an immediate release layer and a delayed, sustained or modified release layer. The immediate layer may release solabegron immediately in the GI tract and the modified, delayed or sustained release layer will release solabegron at a later time and lower in the GI tract. The modified release layer may be coated with either a pH dependent coating or a time dependent coating so as to delay the second release of solabegron to the desired position in the GI tract. It will be understood by one of skill in the art that a bi-layer or multilayer tablet also encompasses a multi compartment tablet or capsule wherein the different regions of pharmaceutical compositions don't have to be in layers, e.g beads compresses within a tablet formation.

In embodiments, the pharmaceutical composition may be a matrix tablet. In embodiments, the matrix tablet may comprise a well-mixed composite of drug(s) with rate-controlling excipients. Numerous sustained and/or delayed release tablets such as membrane controlled system, matrices with water soluble/insoluble polymers, and osmotic systems may be utilized. The tablet may contain either the amorphous form of solabegron, the crystalline form of solabegron or any other form of solabegron. The delayed/sustained release can be achieved by applying a permeable or semipermeable membrane to the tablet core or by mixing the drug with excipient that is either a hydrophilic polymer with high viscosity and gel forming capability or a hydrophobic excipient that slows down the diffusion of drug molecule. An immediate release drug layer can be coated to the tablet that will be available for an early release in the GI tract, while the delayed release core will be designed to delay the drug release after a time period in a designed region of the GI tract. In embodiments, the pharmaceutical composition is a single unit dose. In embodiments, the pharmaceutical composition is two unit doses.

In embodiments, the pharmaceutical composition may be a multicore tablet. In embodiments the multicore tablet may comprise multiple discrete cores consisting of at least one immediate release core and at least one modified release core contained within the same tablet. The at least one immediate release core will be available for an early release in the GI tract, while the at least one modified release core will be designed to delay the drug release after a time period in a designed region of the GI tract. In embodiments, the pharmaceutical composition is a single unit dose. In embodiments, the pharmaceutical composition is two unit doses.

In embodiments, the pharmaceutical composition may be a gastroretentive oral delivery system. In embodiments the gastroretentive oral delivery system may comprise a gastroretentive oral dosage form containing solabegron for the multiple releases of solabegron to a subject in need. The formulation will contain a tablet or capsule having both an immediate release and modified release component. The immediate layer will release solabegron immediately in the GI tract, wherein the modified release layer will release solabegron at a later time inside the GI tract. The gastroretentive oral dosage form may utilize mucoadhesive, swellable, high density or floating technologies to prolong residence time in the stomach thereby allowing a prolonged period for release of both first and second releases in the stomach or upper GI. Both releases may contain any physical form of solabegron such as, for example, amorphous or crystalline solid. In embodiments, the pharmaceutical composition is a single unit dose. In embodiments, the pharmaceutical composition is two unit doses.

In one embodiment, the present application describes a pharmaceutical composition for the delivery of solabegron, comprising at least one immediate release composition, comprising solabegron and at least one pharmaceutically acceptable carrier or diluent; and at least one modified release composition, comprising solabegron and at least one pharmaceutically acceptable carrier or diluent. Further embodiments describe pharmaceutical compositions, wherein the at least one immediate release composition comprises about 75 mg to about 400 mg solabegron. Further embodiments describe pharmaceutical compositions, wherein the at least one modified release composition comprises about 100 mg to about 400 mg solabegron. Further embodiments describe pharmaceutical compositions, wherein the at least one immediate release composition comprises about 100 mg to about 300 mg solabegron and the at least one modified release composition comprises about 100 mg to about 300 mg solabegron. Further embodiments describe pharmaceutical compositions, wherein the at least one immediate release composition comprises about 125 mg solabegron and the at least one modified release composition comprises about 125 mg solabegron. Further embodiments describe pharmaceutical compositions, wherein the at least one immediate release composition comprises about 200 mg solabegron and the at least one modified release composition comprises about 200 mg solabegron. Further embodiments describe pharmaceutical compositions, wherein the at least one immediate release composition comprises about 125 mg solabegron and the at least one modified release composition comprises about 200 mg solabegron. In embodiments, the immediate release of solabegron and the modified release of solabegron may be identical amounts or may be different amounts of solabegron. In embodiments, the immediate release of solabegron may be about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg or about 500 mg. In embodiments the modified release of solabegron may be 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg or about 500 mg. Further embodiments describe pharmaceutical compositions, wherein the at least one immediate release composition achieves a blood plasma Cₘₐₓ from about 0.5 μg/mL to about 3.5 μg/mL. Further embodiments describe pharmaceutical compositions, wherein the at least one modified release composition achieves a blood plasma Cₘₐₓ from about 1.5 μg/mL to about 4 μg/mL. Further embodiments describe pharmaceutical compositions, wherein a Cₘᵢₙ from about 0.25 μg/mL to about 1.5 μg/mL is achieved in about 4 to about 8 hours after administration to a subject in need of treatment. Further embodiments describe pharmaceutical compositions, wherein a Cₘᵢₙ from about 0.01 μg/mL to about 1.0 μg/mL is achieved before about 24 hours after administration to a subject in need of treatment.

Further embodiments describe pharmaceutical compositions, wherein the at least one immediate release composition achieves a blood plasma Cₘₐₓ in about 0.75 to about 4 hours after administration to a subject in need of treatment. Further embodiments describe pharmaceutical compositions, wherein the at least one modified release composition achieves a blood plasma Cₘₐₓ in about 2 to about 8 hours after the first Cₘᵢₙ. Further embodiments describe pharmaceutical compositions, further comprising one or more additional therapeutic agents or treatments useful for the treatment of LUTS, obesity, type 2 diabetes, heart failure, irritable bowel syndrome and similar gastrointestinal disorders, pre-term labor, depression and anxiety, wherein the one more additional therapeutic agents or treatments are selected from the groups consisting of: antimuscarinic agents; alpha adrenoceptor blockers; botulinum toxin; purinergics; cannabinoids; transient receptor potential (TRP) protein inhibitors; prostaglandins; percutaneous tibial nerve stimulation; 5-alpha reductase inhibitors; and phosphodiesterase-5 inhibitors. Further embodiments describe pharmaceutical compositions, wherein the one or more additional therapeutic agents may be administered prior to, simultaneously with, or following the administration of the pharmaceutical composition comprising solabegron.

In one embodiment the present application describes a pharmaceutical composition comprising a therapeutically effective amount of solabegron, wherein the pharmaceutical composition releases at least two releases of solabegron, wherein a first release of solabegron achieves a first target Cₘₐₓ, a second release of solabegron achieves a second target Cₘₐₓ, a first target Cₘᵢₙ is achieved between the first release and the second release and a second Cₘᵢₙ is achieved after the second release. Further embodiments of the present application describe pharmaceutical compositions, wherein said first target Cₘₐₓ is about 1.5 μg/mL to about 4 μg/mL, wherein said first Cₘᵢₙ is about 0.5 μg/mL to about 1.5 μg/mL, wherein said second target Cₘₐₓ is about 1.5 μg.mL to about 4 μg/mL and wherein said second Cₘᵢₙ is about 0.01 μg/mL to about 0.5 μg/mL. Other embodiments of the present application describe pharmaceutical, wherein the first Cₘₐₓ is achieved at about 1 to about 3 hours after the first release, wherein the first Cₘᵢₙ is achieved at about 2 to about 4 hours after the first release, wherein the time between the first target Cₘₐₓ and the second target Cₘₐₓ is about 2 to about 8 hours, wherein the second Cₘₐₓ is achieved at about 1 to about 3 hours after the second release and wherein the second Cₘᵢₙ is achieved at about 3 to about 8 hours after the second release. Further embodiments of the present application describe pharmaceutical compositions, wherein the first release comprises about 100 mg to about 300 mg of solabegron and wherein the second release comprises about 100 mg to about 300 mg of solabegron. Other embodiments of the present application describe pharmaceutical compositions, wherein the concentration of solabegron is about 0.5 μg/mL or below for a period of time from about 12 to about 18 hours. Still other embodiments of the present application describe pharmaceutical compositions, further comprising one or more additional therapeutic agents or treatments useful for the treatment of LUTS, obesity, type 2 diabetes, heart failure, irritable bowel syndrome and similar gastrointestinal disorders, pre-term labor, depression and anxiety, wherein the one more additional therapeutic agents or treatments are antimuscarinic agents, alpha adrenoceptor blockers, botulinum toxin, purinergics, cannabinoids, transient receptor potential (TRP) protein inhibitors, prostaglandins, 5-alpha reductase inhibitors, phosphodiesterase-5 inhibitors or percutaneous tibial nerve stimulation and the one or more additional therapeutic agents may be administered prior to, simultaneously with, or following the administration of the pharmaceutical composition comprising solabegron.

In one embodiment, the present application describes a pharmaceutical composition for the delivery of solabegron, comprising at least one immediate release composition, comprising solabegron and at least one pharmaceutically acceptable carrier or diluent; and at least one modified release composition, comprising solabegron and at least one pharmaceutically acceptable carrier or diluent. Additional embodiments describe pharmaceutical compositions, wherein the at least one immediate release composition comprises about 100 mg to about 300 mg solabegron and the modified release composition comprises about 100 mg to about 300 mg solabegron. Further embodiments describe pharmaceutical compositions, wherein at least one immediate release composition achieves a blood plasma Cₘₐₓ in about 1 to about 3 hours after administration to a subject in need of treatment and at least one modified release composition achieves a blood plasma Cₘₐₓ in about 5 to about 11 hours after administration to a subject in need of treatment. Other embodiments describe pharmaceutical compositions, wherein the at least one immediate release composition achieves a blood plasma Cₘₐₓ from about 1.5 μg/mL to about 4 μg/mL and the at least one modified release composition achieves a blood plasma Cₘₐₓ from about 1.5 μg/mL to about 4 μg/mL. Still additional embodiments describe pharmaceutical compositions, wherein a Cₘᵢₙ from about 0.5 μg/mL to about 1.5 μg/mL is achieved in about 3 to about 5 hours after administration to a subject in need of treatment and a Cₘᵢₙ less than about 0.5 μg/mL is achieved after about 12 hours after administration to a subject in need of treatment. Still other embodiments of the present application describe pharmaceutical compositions, further comprising one or more additional therapeutic agents or treatments useful for the treatment of LUTS, obesity, type 2 diabetes, heart failure, irritable bowel syndrome and similar gastrointestinal disorders, pre-term labor, depression and anxiety, wherein the one more additional therapeutic agents or treatments are antimuscarinic agents, alpha adrenoceptor blockers, botulinum toxin, purinergics, cannabinoids, transient receptor potential (TRP) protein inhibitors, prostaglandins, 5-alpha reductase inhibitors, phosphodiesterase-5 inhibitors or percutaneous tibial nerve stimulation and the one or more additional therapeutic agents may be administered prior to, simultaneously with, or following the administration of the pharmaceutical composition comprising solabegron.

In one embodiment the present application describes a pharmaceutical composition for the delivery of solabegron, comprising at least one immediate release composition, comprising solabegron and at least one pharmaceutically acceptable carrier or diluent; and at least one modified release composition, comprising solabegron and at least one pharmaceutically acceptable carrier or diluent. Further embodiments describe pharmaceutical compositions, wherein the at least one immediate release composition comprises about 75 mg to about 400 mg solabegron. Further embodiments describe pharmaceutical compositions, wherein the at least one modified release composition comprises about 75 mg to about 400 mg solabegron. Further embodiments describe pharmaceutical compositions, wherein the at least one immediate release composition achieves a blood plasma Cₘₐₓ in about 0.75 to about 4 hours after administration to a subject in need of treatment. Further embodiments describe pharmaceutical compositions, wherein the at least one modified release composition achieves a blood plasma Cₘₐₓ in about 6 to about 16 hours after administration to a subject in need of treatment. Further embodiments describe pharmaceutical compositions, further comprising one or more additional therapeutic agents or treatments useful for the treatment of LUTS, obesity, type 2 diabetes, heart failure, irritable bowel syndrome and similar gastrointestinal disorders, pre-term labor, depression and anxiety, wherein the one more additional therapeutic agents or treatments are selected from the groups consisting of: antimuscarinic agents; alpha adrenoceptor blockers; botulinum toxin; purinergics; cannabinoids; transient receptor potential (TRP) protein inhibitors; prostaglandins; percutaneous tibial nerve stimulation; 5-alpha reductase inhibitors; and phosphodiesterase-5 inhibitors. Further embodiments describe pharmaceutical compositions, wherein the one or more additional therapeutic agents may be administered prior to, simultaneously with, or following the administration of the pharmaceutical composition comprising solabegron. Further embodiments describe pharmaceutical compositions, wherein the at least one immediate release composition achieves a blood plasma Cₘₐₓ from about 1.0 μg/mL to about 3.5 μg/mL. Further embodiments describe pharmaceutical compositions, wherein the at least one modified release composition achieves a blood plasma Cₘₐₓ from about 1.5 μg/mL to about 4 μg/mL. Further embodiments describe pharmaceutical compositions, wherein a Cₘᵢₙ from about 0.25 μg/mL to about 1.5 μg/mL is achieved in about 4 to about 8 hours after administration to a subject in need of treatment. Further embodiments describe pharmaceutical compositions, wherein a Cₘᵢₙ from about 0.1 μg/mL to about 1.0 μg/mL is achieved at about 24 hours after administration to a subject in need of treatment. Further embodiments describe pharmaceutical compositions, wherein the at least one immediate release composition comprises about 125 mg solabegron and the at least one modified release composition comprises about 125 mg solabegron. Further embodiments describe pharmaceutical compositions, wherein the at least one immediate release composition comprises about 200 mg solabegron and the at least one modified release composition comprises about 200 mg solabegron. Further embodiments describe pharmaceutical compositions, wherein the at least one immediate release composition comprises about 125 mg solabegron and the at least one modified release composition comprises about 200 mg solabegron.

In one embodiment, the present application describes a pharmaceutical composition comprising a multiparticulate formulation of mini-tablets each comprising a therapeutically effective amount of solabegron, wherein the pharmaceutical composition achieves a first target Cₘₐₓ, a second target Cₘₐₓ, a first target Cₘᵢₙ between the first target Cₘₐₓ and the second target Cₘₐₓ, and a second target Cₘᵢₙ after the second target Cₘₐₓ. Further embodiments describe pharmaceutical compositions, further comprising at least two separate populations of mini-tablets selected from the group consisting of: immediate release mini-tablets; sustained-release mini-tablets; delayed-release mini-tablets; and modified-release mini-tablets. Further embodiments describe pharmaceutical compositions, wherein the mini-tablets are optionally coated. Further embodiments describe pharmaceutical compositions, wherein the mini-tablets comprise solabegron and at least one pharmaceutically acceptable polymer. Further embodiments describe pharmaceutical compositions, wherein the pharmaceutically acceptable polymer is selected from the group consisting of hydroxypropylmethyl cellulose; polyethylene glycol; hydroxypropyl β-cyclodextrin; polyvinyl pyrrolidine; poloxamer; and hydroxypropyl cellulose. Further embodiments describe pharmaceutical compositions, wherein at least one population of mini-tablets further comprises a controlled-release excipient. Further embodiments describe pharmaceutical compositions, wherein at least one population of mini-tablets further comprises an enteric release coating. Further embodiments describe pharmaceutical compositions, wherein the multiparticulate formulation is contained within a capsule.

In embodiments, the present application describes a pharmaceutical composition comprising a modified release solabegron core that is coated in a matrix of immediate/early release solabegron and a polymer, comprising a therapeutically effective amount of solabegron, wherein the pharmaceutical composition achieves a first target Cₘₐₓ, a second target Cₘₐₓ, a first target Cₘᵢₙ between the first target Cₘₐₓ and the second target Cₘₐₓ, and a second target Cₘᵢₙ after the second target Cₘₐₓ. In further embodiments the modified release solabegron core comprises solabegron and at least one excipient surrounded by a delayed release layer. In further embodiments the modified release core comprises micronized solabegron, mannitol, microcrystalline cellulose, croscarmellose sodium, citric acid, sodium lauryl sulfate, colloidal SiO2, and sodium stearyl fumarate; surrounded by a delayed release layer comprising Opadry Clear, Eudragit L30 D55, and Plasacryl HTP20. In embodiments the matrix of solabegron and a polymer comprises solabegron and opadry. In further embodiments the solabegron is the HCl salt of solabegron.

In one embodiment, the present application describes the amorphous form of solabegron. An amorphous form is a solid that does not have a definite geometric or crystalline shape. It is a solid in which there is no long-range order in the positions of the atoms that is characteristic or a crystal. For certain compounds, the amorphous form of the compound exhibits desirable properties for purposes of formulation and bioavailability. Fourier-transform infrared spectroscopy (FTIR) scans of solabegron API and a spray-dried 20:80 solabegron to polyvinyl pyrrolidine (PVPK30) formulation were conducted. The solabegron API displays amine (N—H) peaks at about 3400 and 3250 cm⁻¹ and carbonyl peaks at 1593 cm⁻¹. The spray-dried 20:80 solabegron to polyvinyl pyrrolidine (PVPK30) formulation displays a broadening of both the amine and carbonyl peaks. The observed changes in the amine and carbonyl peaks in the solabegron-PVPK30 formulation are indicative of the amorphous form of solabegron.

In one embodiment, the present application describes a pharmaceutical composition comprising a therapeutically effective amount of the amorphous form of solabegron and at least one pharmaceutically acceptable carrier or diluent. Further embodiments describe pharmaceutical compositions, wherein the therapeutically effective amount of amorphous solabegron is about 150 mg to about 850 mg. Further embodiments describe pharmaceutical compositions, wherein the pharmaceutical composition is prepared by spray-drying the therapeutically effective amount of amorphous solabegron with at least one pharmaceutically acceptable polymer. Further embodiments describe pharmaceutical compositions, wherein the pharmaceutically acceptable polymer is selected from the group consisting of hydroxypropylmethyl cellulose; polyethylene glycol; hydroxypropyl β-cyclodextrin; polyvinyl pyrrolidine; poloxamer; and hydroxypropyl cellulose. Further embodiments describe pharmaceutical compositions, wherein the pharmaceutical composition is prepared by hot-melt extruding the therapeutically effective amount of amorphous solabegron with at least one pharmaceutically acceptable polymer. Further embodiments describe pharmaceutical compositions, wherein the pharmaceutically acceptable polymer is selected from the group consisting of hydroxypropylmethyl cellulose; polyethylene glycol; hydroxypropyl β-cyclodextrin; polyvinyl pyrrolidine; poloxamer; and hydroxypropyl cellulose.

The pharmaceutical compositions of the present application can be administered transdermally, orally, by inhalation, nasally, or parenterally, such as subcutaneously or intravenously, as well as sublingually to various mammalian species known to be subject to such maladies, e.g., humans, in an effective amount up to about 1 gram, preferably up to about 800 mg, more preferably up to about 600 mg in a once-a-day regimen. In some embodiments the pharmaceutical compositions of the present invention are administered in an effective up of about 175 mg to about 650 mg. In some embodiments the pharmaceutical compositions of the present invention are administered in an effective up of about 150 mg to about 500 mg.

The pharmaceutical compositions of the present application can be administered for any of the uses described herein by any suitable means, for example, orally, such as in the form of tablets, capsules, granules or powders; sublingually; bucally; parenterally, such as by subcutaneous, intravenous, intramuscular, or intrasternal injection or infusion techniques (e.g., as sterile injectable aqueous or non-aqueous solutions or suspensions); in dosage unit formulations containing non-toxic, pharmaceutically acceptable vehicles or diluents. The present compositions can, for example, be administered in a form suitable for immediate release or extended release. Immediate release or extended release can be achieved by the use of suitable pharmaceutical compositions comprising the present compounds, or, particularly in the case of extended release, by the use of devices such as subcutaneous implants or osmotic pumps. Other devices include vaginal rings. The present compositions can also be administered liposomally.

The formulation for a beta-3 adrenoceptor agonist can significantly modify the absorption profile. For example, some compounds are differentially absorbed in different regions of the GI tract. Some of the factors involved in absorption can include pH-dependent solubility, particle size, lipophilicity, ionization, GI-motility or transporters. In the current example for the absorption of solabegron, solabegron demonstrates pH-dependent solubility and absorption. Accordingly, solabegron and pharmaceutical salts thereof display the optimum absorption in the proximal GI tract. Pharmaceutical compositions are presented herein that improve the pH-dependent solubility of solabegron in the distal GI tract. Under these improved conditions, a second release of solabegron and absorption will result. Additionally, methods for the release of solabegron in the distal GI tract based on pH are presented herein.

Another example of producing a delayed second release is based on the transit time of the dosage form. This is achievable through the time-dependent erosion or dissolution of the dosage form coating. The GI transit time is well understood, and the coatings are designed to erode within a specific time range that corresponds to a specific region within the GI tract. Pharmaceutical compositions and methods of use are presented herein for the release of solabegron based on time-dependent erosion.

Exemplary compositions for oral administration include emulsions and suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate and/or lactose and/or other excipients, binders, extenders, disintegrants, diluents and lubricants such as those known in the art. Surfactants, oils and co-solvents that can be used in oral solabegron compositions include, but are not limited to: Tween 80, Span 80, Cremophor EL, Solutol HS15, Vitamin E GTPS, Poloxamer 407, Labrasol, Labrafils, Gelucire 44/14, Lauroglycol, Capryol 90, Ethanol, Benzyl Alcohol, Proylene Glycol (PG), PEG400, PEG1000, Poloxamer 188, Glycerin, Capmul MCM, Peceol, Mannitol, Microcrystalline Cellulose, Kolliphor P188, Croscarmellose Sodium, Citric Acid, Sodium Lauryl Sulfate, Colloidal SiO2, Sodium Stearyl Fumarate, Opadry Clear, Eudragit L30 D55, Plasacryl HTP20, solabegron HCl, and Opadry Clear.

The compositions of the present application can also be delivered through the oral cavity by sublingual and/or buccal administration. Molded tablets, compressed tablets or freeze-dried tablets are exemplary forms which may be used. Exemplary compositions include those formulating the present beta-3 adrenoceptor agonists with fast dissolving diluents such as mannitol, lactose, sucrose and/or cyclodextrins.

The compositions of the present application may take the form of pulsatile delivery systems such as, for example, PULSINCAP®, MICROPUMP®, MEDUSA™, PORT® system, CHRONOTROPIC®, TIME CLOCK®, multilayered tablets, DiffuCORE®, rupturable tablets, ACCU-BREAK® system, DIFFUCAPS®, DIFFUTABS®, Eurand MINITABS®, MICROCAPS®, SODAS®, IPDAS®, OsDrC®, OptiDose™, OptiMelt™, ZYDIS®, CODAS®, PRODAS®, TMDS®, DMDS®, PMDS®, GEOCLOCK®, GEOMATRIX®, PULSYS®, OROS® INTELLIMATRIX™ and VERSETROL™. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use.

In some embodiments, the composition of the present invention comprises a modified release solabegron core that is coated in a matrix of solabegron and a polymer. In further embodiments the modified release solabegron core comprises solabegron and at least one excipient surrounded by a delayed release layer. In further embodiments the modified release core comprises micronized solabegron, mannitol, microcrystalline cellulose, croscarmellose sodium, citric acid, sodium lauryl sulfate, colloidal SiO2, and sodium stearyl fumarate; surrounded by a delayed release layer comprising Opadry Clear, Eudragit L30 D55, and Plasacryl HTP20. In embodiments the matrix of solabegron and a polymer comprises solabegron and Opadry. In further embodiments the solabegron is the HCl salt of solabegron.

In some embodiments the pharmaceutical composition of the present invention comprises:

| | Percent | Chemical Name |
|---|---|---|
| Tablet | About 25 to about 40 weight %* | Solabegron |
| Core | About 20 to about 50 weight % | Diluent |
| | About 5 to about 15 weight % | Bulking Agent |
| | About 0 to about 10 weight % | 1^{st} Surfactant/ |
| | | Wetting Agent |
| | About 5 to about 15 weight % | Disintegrant |
| | About 0 to about 5 weight % | Solubilizer |
| | About 0 to about 5 weight % | 2^{nd} Surfactant/ |
| | | Wetting Agent |
| | About 0 to about 2 weight % | Flow Enhancer |
| | About 0 to about 5 weight % | Lubricant |
| Modified | About 0 to about 5 weight % | Coating Diluent |
| Release | About 3 to about 5 weight % | >pH 5.5 Delayed |
| Layer | | Release Polymer |
| | About 0 to about 2 weight % | Plasticizer |
| Immediate | About 5 to about 15 weight %* | Solabegron |
| Release | About 5 to about 15 weight % | Coating Diluent |
| Layer | | |

*adjusted for HCl salt if the solabegron is the HCl salt form

It will be understood by one of skill in the art that the total of all of the components in the above composition must equal 100%. In further embodiments the solabegron is the solabegron salt. In further embodiments the solabegron in the immediate release layer is a micronized solabegron. In some embodiments the solabegron is about 25 weight %, about 26 weight %, about 27 weight %, about 28 weight %, about 29 weight %, about 30 weight %, about 31 weight %, about 32 weight %, about 33 weight %, about 34 weight %, about 35 weight %, about 36 weight %, about 37 weight %, about 38 weight %, about 39 weight %, or about 40 weight %. In some embodiments the first surfactant/wetting agent is 0 weight %, 1 weight %, 2 weight %, 3 weight %, 4 weight %, 5 weight %, 6 weight %, 7 weight %, 8 weight %, 9 weight % or, 10 weight %. In some embodiments the second surfactant/wetting agent is 0 weight %, 1 weight %, 2 weight %, 3 weight %, 4 weight %, or 5 weight %.

In some embodiments the pharmaceutical composition of the present invention comprises:

| | Percent | Chemical Name |
|---|---|---|
| Tablet | About 25 to about 40 weight %* | Micronized Solabegron HCl |
| Core | | (D90 = ~4 um,) |
| | About 20 to about 50 weight % | Mannitol |
| | About 5 to about 15 weight % | Microcrystalline Cellulose |
| | About 0 to about 10 weight % | Kolliphor P188 (aka |
| | | Poloxamer 188) |
| | About 5 to about 15 weight % | Croscarmellose Sodium |
| | About 0 to about 5 weight % | Citric Acid |
| | About 0 to about 5 weight % | Sodium Lauryl Sulfate |
| | About 0 to about 2 weight % | Colloidal SiO2 |
| | About 0 to about 5 weight % | Sodium Stearyl Fumarate |
| Modified | About 0 to about 5 weight % | Sub-total |
| Release | About 3 to about 5 weight % | Opadry Clear |
| Layer | About 0 to about 2 weight % | Eudragit L30 D55 |
| Immediate | About 5 to about 15 weight %* | Solabegron HCl |
| Release | About 5 to about 15 weight % | Opadry Clear |
| Layer | | |

*adjusted for HCl salt

It will be understood by one of skill in the art that the total of all of the components in the above composition must equal 100%. In some embodiments the solabegron is about 25 weight %, about 26 weight %, about 27 weight %, about 28 weight %, about 29 weight %, about 30 weight %, about 31 weight %, about 32 weight %, about 33 weight %, about 34 weight %, about 35 weight %, about 36 weight %, about 37 weight %, about 38 weight %, about 39 weight %, or about 40 weight %. In some embodiments the kolliphor P188 is 0 weight %, 1 weight %, 2 weight %, 3 weight %, 4 weight %, 5 weight %, 6 weight %, 7 weight %, 8 weight %, 9 weight % or, 10 weight %. In some embodiments the sodium lauryl sulfate is 0 weight %, 1 weight %, 2 weight %, 3 weight %, 4 weight %, or 5 weight %.

In one embodiment the pharmaceutical composition comprises:

| | % w/w | Chemical Name |
|---|---|---|
| Tablet | about 22.8* | Micronized Solabegron HCl |
| Core | | (D90 = ~4 um) |
| | about 31.4 | Mannitol |
| | about 7.6 | Microcrystalline Cellulose |
| | about 0 to about 10 | Poloxamer 188 (aka Kolliphor 188) |
| | about 5 to about 15 | Croscarmellose Sodium |
| | about 2.3 | Citric Acid |
| | about 0 to about 5 | Sodium Lauryl Sulfate |
| | about 0.4 | Colloidal SiO2 |
| | about 1.6 | Sodium Stearyl Fumarate |
| DR | about 2.3 | Opadry Clear |
| Layer | about 3.8 | Eudragit L30 D55 |
| | about 0.9 | Plasacryl HTP20 |
| IR | about 8.5* | Solabegron HCl |
| Layer | about 8.5 | Opadry Clear |

*adjusted for HCl salt

It will be understood by one of skill in the art that the total of all of the components in the above composition must equal 100%.

In one embodiment the pharmaceutical composition comprises:

| | % w/w | Chemical Name |
|---|---|---|
| Tablet | About 22.8* | Micronized Solabegron HCl |
| Core | | (D90 = ~4 um) |
| | About 31.4 | Mannitol |
| | About 7.6 | Microcrystalline Cellulose |
| | About 7.5 | Croscarmellose Sodium |
| | About 2.3 | Citric Acid |
| | About 2.3 | Sodium Lauryl Sulfate |
| | About 0.4 | Colloidal SiO2 |
| | About 1.6 | Sodium Stearyl Fumarate |
| | About 76 | Sub-total |
| DR | About 2.3 | Opadry Clear |
| Layer | About 3.8 | Eudragit L30 D55 |
| | About 0.9 | Plasacryl HTP20 |
| | About 82.9 | Sub-total |
| IR | About 8.5* | Solabegron HCl |
| Layer | About 8.5 | Opadry Clear |

*adiusted for HCl salt

It will be understood by one of skill in the art that the total of all of the components in the above composition must equal 100%.

In one embodiment the pharmaceutical composition comprises:

| | % w/w | Chemical Name |
|---|---|---|
| Tablet | About 22.7* | Micronized Solabegron HCl |
| Core | | (D90 = ~4 um) |
| | About 26.2 | Mannitol |
| | About 7.6 | Microcrystalline Cellulose |
| | About 7.6 | Poloxamer 188 (aka Kolliphor 188) |
| | About 7.6 | Croscarmellose Sodium |
| | About 2.3 | Citric Acid |
| | About 0.4 | Colloidal SiO2 |
| | About 1.6 | Sodium Stearyl Fumarate |
| | About 76 | Sub-total |
| DR | About 2.3 | Opadry Clear |
| Layer | About 3.8 | Eudragit L30 D55 |
| | About 0.9 | Plasacryl HTP20 |
| | About 82.9 | Sub-total |
| IR | About 8.5* | Solabegron HCl |
| Layer | About 8.5 | Opadry Clear |

*adjusted for HCl salt

It will be understood by one of skill in the art that the total of all of the components in the above composition must equal 100%.

In one embodiment the pharmaceutical composition comprises:

| | % w/w | Chemical Name |
|---|---|---|
| Tablet | About 22.7* | Micronized Solabegron HCl |
| Core | | (D90 = ~4 um) |
| | About 23.9 | Mannitol |
| | About 7.6 | Microcrystalline Cellulose |
| | About 7.6 | Poloxamer 188 (aka Kolliphor 188) |
| | About 7.6 | Croscarmellose Sodium |
| | About 2.3 | Citric Acid |
| | About 2.3 | Sodium Lauryl Sulfate |
| | About 0.4 | Colloidal SiO2 |
| | About 1.6 | Sodium Stearyl Fumarate |
| | About 76 | Sub-total |
| DR | About 2.3 | Opadry Clear |
| Layer | About 3.8 | Eudragit L30 D55 |
| | About 0.9 | Plasacryl HTP20 |
| | About 82.9 | Sub-total |
| IR | About 8.5* | Solabegron HCl |
| Layer | About 8.5 | Opadry Clear |

*adjusted for HCl salt

It will be understood by one of skill in the art that the total of all of the components in the above composition must equal 100%.

Exemplary compositions for parenteral administration include injectable solutions or suspensions which can contain, for example, suitable non-toxic, parenterally acceptable diluents or solvents, such as mannitol, 1,3-butanediol, water, polyethylene glycol, Ringer's solution, an isotonic sodium chloride solution, or other suitable dispersing or wetting and suspending agents, including synthetic mono- or diglycerides, and fatty acids, including oleic acid, or Cremaphor.

Exemplary compositions for transdermal administration include transdermal therapeutic systems (hereinafter “TTS”). TTS are patches having a layered structure and comprising at least one active pharmaceutical ingredient in a reservoir layer. A distinction is made between matrix-type and reservoir-type TTS: in the first case the reservoir layer containing the active pharmaceutical ingredient has a pressure-sensitive adhesive finish, and in the second case a membrane which controls the rate of release of the active pharmaceutical ingredient, and where appropriate an additional pressure-sensitive adhesive layer, are present.

Exemplary compositions for delivery directly to the bladder include extended-release solid-drug core devices that are implanted via catheter.

The therapeutic agents in the pharmaceutical compositions of the present application may exist in any physical form known to one of skill in the art such as, for example, nanoparticles, crystalline solids, amorphous solids, polymorphs, ionic solids such as, for example, cations, anions and zwitterions, pharmaceutically acceptable salts, hydrates, solvates, stereoisomers, solutions and suspensions. Crystalline solids have regular ordered arrays of components held together by uniform intermolecular forces, whereas the components of amorphous solids are not arranged in regular arrays. Hydrates are substances that incorporate at least one water molecule into their crystalline matrix. Solvates are substances that incorporate at least one solvent molecule into their crystalline matrix. Polymorphs exhibit different crystalline structures for molecules that have the same molecular formula and sequence of bonded atoms. Stereoisomers are isomeric molecules that have the same molecular formula and sequence of bonded atoms (constitution), but that differ only in the three-dimensional orientations of their atoms in space.

Further, the therapeutic agents in the pharmaceutical compositions of the present application may exist in any isotopic form known to one of skill in the art such as, for example, deuterated, tritiated, ¹³C, ¹⁴C, etc.

The present application describes immediate release, extended release, delayed release, and modified release formulations of solabegron. Previously, both the HCL salt and the amorphous form of solabegron have been tested in clinical studies and based on the totality of the in-vitro and clinical data there were found to be two impediments to developing a solabegron QD formulation to provide the desired PK profile illustrated in FIG. 1: 1) the pH dependent and low solubility of solabegron and 2) the position of release of solabegron in the intestine. Compositions were prepared to overcome the above-described impediments in developing a solabegron QD formulation to provide the desired PK profile of FIG. 1.

It will be understood that the specific dose level and frequency of dosage for any particular subject can be varied and will depend upon a variety of factors including the species, age, body weight, general health, gender and diet of the subject, the mode and time of administration, rate of excretion, drug combination, and severity of the particular condition.

The present application includes within its scope pharmaceutical compositions comprising, a therapeutically effective amount of solabegron, alone or in combination with a pharmaceutical carrier or diluent. Surfactants, oils and co-solvents that can be used in solabegron compositions include, but are not limited to: Tween 80, Span 80, Cremophor EL, Solutol HS15, Vitamin E GTPS, Poloxamer 407, Labrasol, Labrafils, Gelucire 44/14, Lauroglycol, Capryol 90, Ethanol, Benzyl Alcohol, Proylene Glycol (PG), PEG400, PEG1000, Poloxamer 188, Glycerin, Capmul MCM, Peceol, Mannitol, Microcrystalline Cellulose, Kolliphor P188, Croscarmellose Sodium, Citric Acid, Sodium Lauryl Sulfate, Colloidal SiO2, Sodium Stearyl Fumarate, Opadry Clear, Eudragit L30 D55, Plasacryl HTP20, solabegron HCl, and Opadry Clear.

Optionally, the pharmaceutical compositions of the present invention can be used alone, or in combination with other suitable therapeutic agents or treatments useful in the treatment of LUTS including: antimuscarinic agents, alpha adrenoceptor blockers, botulinum toxin, purinergics, cannabinoids, transient receptor potential (TRP) protein inhibitors, prostaglandins, percutaneous tibial nerve stimulation, 5-alpha reductase inhibitors and phosphodiesterase-5 inhibitors.

Optionally, the pharmaceutical compositions of the present invention can be used alone, or in combination with other suitable therapeutic agents or treatments useful in the treatment of obesity, diabetes, heart failure, irritable bowel syndrome (IBS), preterm labor, anxiety or depression.

Such other therapeutic agent(s) may be administered prior to, simultaneously with, or following the administration of solabegron containing pharmaceutical composition in accordance with the invention.

Examples of suitable antimuscarinic agents for use in combination with the pharmaceutical compositions of the present application include tolterodine, oxybutynin, trospium, solifenacin, darifenacin, propiverine, fesoterodine, and pharmaceutically acceptable salts thereof.

Examples of suitable alpha adrenoceptor blockers for use in combination with the pharmaceutical compositions of the present application include tamuslosin, alfuzosin, and silodosin.

Examples of suitable 5-alpha reductase inhibitors for use in combination with the pharmaceutical compositions of the present application include finasteride, dutaseteride and pharmaceutically acceptable salts thereof.

Examples of suitable phosphodiesterase-5 inhibitors for use in combination with the pharmaceutical compositions of the present application include sildenafil, tadalafil, vardenafil, udenafil, avanafil and pharmaceutically acceptable salts thereof.

Examples of suitable therapeutics for obesity: orlistat (Xenical®), lorcaserin (Belviq®), phentermine and topiramate (Qsymia®), buproprion and naltrexone (Contrave®), and uncertain mimetics such as liraglutide (Saxenda®).

Examples of suitable therapeutics for diabetes: metformin, sulfonylureas (DiaBeta®, Glynase®), glipizide (Glucotrol®) glimepiride (Amaryl®), meglitinides, repaglinide (Prandin®), nateglinide (Starlix®), thiazolidinedione (Actos®, Avandia®), DPP-4 inhibitors, sitagliptin (Januvia®), saxagliptin (Onglyza®), linagliptin (Tradjenta®), purinergics, GLP-1 receptor agonists exenatide (Byetta®), liraglutide (Victoza®), SGLT2 inhibitors, canagliflozin (Invokana®), dapagliflozin (Farxiga®) and insulin.

Examples of suitable therapeutics for heart failure: angiotensin-converting enzyme (ACE) inhibitors, enalapril, lisinopril, angiotensin II receptor blockers, (Losartan®, (Valsartan®), beta blockers)(Carvedilol®), metoprolol, bisoprolol, diuretics, hydrochlorthiazide, furosemide, aldosterone antagonists, spironolactone, eplerenone (Inspra®), inotropes and digoxin

Examples of suitable therapeutics for IBS: alosetron (Lotronex®), lubiprostone (Amitiza®), eluxadoline (Viberzi®), llinaclotide (Linzess®), rifaximin (Xifaxan®), fiber supplements (OTC), psyllium (Metamucil®), methylcellulose (Citrucel®), anti-diarrheal medications, loperamide (Imodium®), bile acid binders, cholestyramine (Prevalite®), colestipol (Colestid®), colesevelam (Welchol®), anticholinergic and antispasmodic medications, (Levsin®) and dicyclomine (Bentyl®).

Examples of suitable therapeutics for preterm labor: tocolytics, magnesium sulfate, corticosteroids, terbutaline, ritodrine, nifedipine, oxytocin receptor antagonists (Atosiban®).

Examples of suitable therapeutics for anxiety: escitalopram (Lexapro®), duloxetine (Cymbalta®), venlafaxine (Effexor XR®) and paroxetine (Paxil®), buspirone benzodiazepines alprazolam (Xanax®), diazepam (Valium®) and lorazepam (Ativan®).

Examples of suitable therapeutics for depression: selective serotonin reuptake inhibitors (SSRIs), fluoxetine (Prozac®), paroxetine (Paxil®, Pexeva®), sertraline (Zoloft®), citalopram (Celexa®), escitalopram (Lexapro®), serotonin-norepinephrine reuptake inhibitors (SNRIs), duloxetine (Cymbalta®), venlafaxine (Effexor XR®), desvenlafaxine (Pristiq®, Khedezla®), levomilnacipran (Fetzima®), norepinephrine-dopamine reuptake inhibitors (NDRIs), bupropion (Wellbutrin®, Aplenzin®, Forfivo XL®), atypical antidepressants, trazodone and mirtazapine (Remeron®), vortioxetine)(Brintellix®), vilazodone)(Viibryd®), vilazodone, tricyclic antidepressants, imipramine (Tofranil®), nortriptyline (Pamelor®), amitriptyline, doxepin, trimipramine (Surmontil®), desipramine (Norpramin®), protriptyline (Vivactil®), monoamine oxidase inhibitors (MAOIs), tranylcypromine (Parnate®), phenelzine (Nardil®) and isocarboxazid (Marplan®).

### Methods of Treatment

In embodiments, methods of treating a disease comprising administering to a subject in need thereof a pharmaceutical composition as described herein are provided. In embodiments, the disease may be LUTS, obesity, type 2 diabetes, heart failure, irritable bowel syndrome and similar gastrointestinal disorders, pre-term labor, depression and anxiety, combinations thereof. In embodiments, treating LUTS may include treating or otherwise decreasing frequency of urgency, decreasing nocturia, decreasing urinary micturition frequency, decreasing urinary incontinence, increasing voided volume, decreasing post-void residual volume, and/or improving subject reporting outcomes. In embodiments, the pharmaceutical composition may be administered once a day. In embodiments, the pharmaceutical composition is administered every other day (QOD), once a day (QD), twice a day (BID) or three times a day (TID) to a subject in need thereof.

In embodiments, methods of treating such diseases may further comprise administering a therapeutically effective amount of one or more additional therapeutic agents. In embodiments, the one or more additional therapeutic agents may be administered prior to, simultaneously with, or following the administration of the pharmaceutical composition comprising solabegron. In embodiments, the one or more additional therapeutic agents may be an antimuscarinic agents, alpha adrenoceptor blockers, botulinum toxin, purinergics, cannabinoids, transient receptor potential (TRP) protein inhibitors, prostaglandins, 5-alpha reductase inhibitors, phosphodiesterase-5 inhibitors or percutaneous tibial nerve stimulation. In embodiments, the antimuscarinic agent may be tolterodine, oxybutynin, trospium, solifenacin, darifenacin, propiverine, fesoterodine, and pharmaceutically acceptable salts thereof as described in the pharmaceutical composition section above and in paragraphs [0127] through [0139].

In one embodiment, the present application describes a method for treating LUTS, obesity, type 2 diabetes, heart failure, irritable bowel syndrome and similar gastrointestinal disorders, pre-term labor, depression and anxiety, comprising administering a pharmaceutical composition for the delivery of solabegron, comprising an immediate release composition, comprising solabegron and at least one pharmaceutically acceptable carrier or diluent; and a modified release composition, comprising solabegron and at least one pharmaceutically acceptable carrier or diluent to a subject in need thereof. Further embodiments describe methods, wherein the subject achieves a blood plasma Cₘₐₓ of about 0.5 μg/mL to about 3.5 μg/mL in about 0.75 to about 4 hours after administration. Further embodiments describe methods, wherein the subject achieves a blood plasma Cₘᵢₙ from about 0.25 μg/mL to about 1.5 μg/mL in about 4 to about 8 hours after administration. Further embodiments describe methods, wherein the subject achieves a blood plasma Cₘₐₓ of about 1.5 μg/mL to about 4 μg/mL in about 2 to about 8 hours after the first Cₘᵢₙ. Further embodiments describe methods, wherein the subject achieves a blood plasma Cₘᵢₙ from about 0.01 μg/mL to about 1.0 μg/mL before about 24 hours after administration. Further embodiments describe methods, further comprising administering one or more additional therapeutic agents or treatments useful for the treatment of LUTS, obesity, type 2 diabetes, heart failure, irritable bowel syndrome and similar gastrointestinal disorders, pre-term labor, depression and anxiety, wherein the one more additional therapeutic agents or treatments are selected from the groups consisting of: antimuscarinic agents; alpha adrenoceptor blockers; botulinum toxin; purinergics; cannabinoids; transient receptor potential (TRP) protein inhibitors; prostaglandins; percutaneous tibial nerve stimulation; 5-alpha reductase inhibitors; and phosphodiesterase-5 inhibitors. Further embodiments describe method, wherein the one or more additional therapeutic agents may be administered prior to, simultaneously with, or following the administration of the pharmaceutical composition comprising solabegron. Further embodiments describe methods, wherein the pharmaceutical composition is administered once a day to a subject in need thereof.

In one embodiment, the present application describes a method for treating LUTS, obesity, type 2 diabetes, heart failure, irritable bowel syndrome and similar gastrointestinal disorders, pre-term labor, depression and anxiety, comprising administering a pharmaceutical composition, comprising a therapeutically effective amount of solabegron, wherein the pharmaceutical composition releases at least two releases of solabegron, wherein a first release of solabegron achieves a first target Cₘₐₓ, a second release of solabegron achieves a second target Cₘₐₓ, a first target Cₘᵢₙ is achieved between the first release and the second release and a second Cₘᵢₙ is achieved after the second release. Further embodiments describe methods, wherein said first target Cₘₐₓ is about 0.5 μg/mL to about 3.5 μg/mL. Further embodiments describe methods, wherein said second target Cₘₐₓ is about 1.5 μg/mL to about 4 μg/mL. Further embodiments describe methods, wherein said first Cₘᵢₙ is about 0.25 μg/mL to about 1.5 μg/mL. Further embodiments describe methods, wherein said second Cₘᵢₙ is about 0.01 μg/mL to about 1.0 μg/mL. Further embodiments describe methods, further comprising administering one or more additional therapeutic agents or treatments useful for the treatment of LUTS, obesity, type 2 diabetes, heart failure, irritable bowel syndrome and similar gastrointestinal disorders, pre-term labor, depression and anxiety, wherein the one more additional therapeutic agents or treatments are selected from the groups consisting of: antimuscarinic agents; alpha adrenoceptor blockers; botulinum toxin; purinergics; cannabinoids; transient receptor potential (TRP) protein inhibitors; prostaglandins; percutaneous tibial nerve stimulation; 5-alpha reductase inhibitors; and phosphodiesterase-5 inhibitors. Further embodiments describe methods, wherein the one or more additional therapeutic agents may be administered prior to, simultaneously with, or following the administration of the pharmaceutical composition comprising solabegron. Further embodiments describe methods, wherein the pharmaceutical composition is administered once a day to a subject in need thereof.

In one embodiment the present application describes a method for treating LUTS, obesity, type 2 diabetes, heart failure, irritable bowel syndrome and similar gastrointestinal disorders, pre-term labor, depression and anxiety, comprising administering a pharmaceutical composition for the delivery of solabegron, comprising an immediate release composition, comprising solabegron and at least one pharmaceutically acceptable carrier or diluent; and a modified release composition, comprising solabegron and at least one pharmaceutically acceptable carrier or diluent to a subject in need thereof. Additional embodiments describe methods, wherein the subject achieves a blood plasma Cₘₐₓ of about 1.5 μg/mL to about 4 μg/mL in about 1 to about 3 hours after administration. Further embodiments describe methods, wherein the subject achieves a blood plasma Cₘᵢₙ from about 0.5 μg/mL to about 1.5 μg/mL in about 3 to about 5 hours after administration. Still further embodiments describe methods, wherein the subject achieves a blood plasma Cₘₐₓ of about 1.5 μg/mL to about 4 μg/mL in about 5 to about 11 hours after administration. Additional embodiments describe methods, wherein the subject achieves a blood plasma Cₘᵢₙ less than about 0.5 μg/mL after about 12 hours after administration. Still additional embodiments describe methods, wherein the pharmaceutical composition is administered every other day (QOD), once a day (QD), twice a day (BID) or three times a day (TID) to a subject in need thereof. Still other embodiments of the present application describe pharmaceutical compositions, further comprising one or more additional therapeutic agents or treatments useful for the treatment of LUTS, obesity, type 2 diabetes, heart failure, irritable bowel syndrome and similar gastrointestinal disorders, pre-term labor, depression and anxiety, wherein the one more additional therapeutic agents or treatments are antimuscarinic agents, alpha adrenoceptor blockers, botulinum toxin, purinergics, cannabinoids, transient receptor potential (TRP) protein inhibitors, prostaglandins, 5-alpha reductase inhibitors, phosphodiesterase-5 inhibitors or percutaneous tibial nerve stimulation and the one or more additional therapeutic agents may be administered prior to, simultaneously with, or following the administration of the pharmaceutical composition comprising solabegron.

In one embodiment the present application describes a method for treating LUTS, obesity, type 2 diabetes, heart failure, irritable bowel syndrome and similar gastrointestinal disorders, pre-term labor, depression and anxiety, comprising administering a pharmaceutical composition, comprising a therapeutically effective amount of solabegron, wherein the pharmaceutical composition releases at least two releases of solabegron, wherein a first release of solabegron achieves a first target Cₘₐₓ a second release of solabegron achieves a second target Cₘₐₓ, a first target Cₘᵢₙ is achieved between the first release and the second release and a second Cₘᵢₙ is achieved after the second release. Additional embodiments describe methods, wherein said first target Cₘₐₓ is about 1.5 μg/mL to about 4 μg/mL, said second target Cₘₐₓ is about 1.5 μg.mL to about 4 μg/mL, said first Cₘᵢₙ is about 0.5 μg/mL to about 1.5 μg/mL and said second Cₘᵢₙ is about 0.01 μg/mL to about 0.5 μg/mL. Still other embodiments of the present application describe pharmaceutical compositions, further comprising one or more additional therapeutic agents or treatments useful for the treatment of LUTS, obesity, type 2 diabetes, heart failure, irritable bowel syndrome and similar gastrointestinal disorders, pre-term labor, depression and anxiety, wherein the one more additional therapeutic agents or treatments are antimuscarinic agents, alpha adrenoceptor blockers, botulinum toxin, purinergics, cannabinoids, transient receptor potential (TRP) protein inhibitors, prostaglandins, 5-alpha reductase inhibitors, phosphodiesterase-5 inhibitors or percutaneous tibial nerve stimulation and the one or more additional therapeutic agents may be administered prior to, simultaneously with, or following the administration of the pharmaceutical composition comprising solabegron. Still further embodiments describe methods, wherein the pharmaceutical composition is administered every other day (QOD), once a day (QD), twice a day (BID) or three times a day (TID) to a subject in need thereof.

In one embodiment the present application describes a method of treating one or more symptoms of OAB, comprising administering a pharmaceutical composition, comprising a therapeutically effective amount of solabegron and at least one pharmaceutically acceptable diluent or carrier, wherein the one or more symptoms of OAB are selected from the group consisting of: frequency of urinary urgency; nocturia; increase in urinary micturition frequency; and urinary incontinence. Further embodiments describe methods, wherein the pharmaceutical composition may be administered in the morning or the pharmaceutical composition may be administered with a meal. Additional embodiments describe methods, wherein the improvement in the one or more symptoms of over active bladder is increased bladder volume as measured by void volume.

In one embodiment, the present application describes a once-daily treatment for LUTS, obesity, type 2 diabetes, heart failure, irritable bowel syndrome and similar gastrointestinal disorders, pre-term labor, depression and anxiety that achieves a desired blood plasma Cₘₐₓ while also not desensitizing the beta-3 adrenoceptor, comprising a pharmaceutical composition, comprising a therapeutically effective amount of solabegron, wherein the pharmaceutical composition releases at least two releases of solabegron, wherein a first release of solabegron achieves a first target Cₘₐₓ, a second release of solabegron achieves a second target Cₘₐₓ, a first target Cₘᵢₙ is achieved between the first release and the second release and a second Cₘᵢₙ is achieved after the second release. Further embodiments describe once-daily treatments, wherein said first target Cₘₐₓ is about 0.5 μg/mL to about 3.5 μg/mL. Further embodiments describe once-daily treatments, wherein said second target Cₘₐₓ is about 1.5 μg.mL to about 4 μg/mL., wherein said first Cₘᵢₙ is about 0.25 μg/mL to about 1.5 μg/mL. Further embodiments describe once-daily treatments, wherein said second Cₘᵢₙ is from about 0.01 μg/mL to about 1.0 μg/mL. Further embodiments describe once-daily treatments, further comprising administering one or more additional therapeutic agents or treatments useful for the treatment of LUTS, obesity, type 2 diabetes, heart failure, irritable bowel syndrome and similar gastrointestinal disorders, pre-term labor, depression and anxiety, wherein the one more additional therapeutic agents or treatments are selected from the groups consisting of: antimuscarinic agents; alpha adrenoceptor blockers; botulinum toxin; purinergics; cannabinoids; transient receptor potential (TRP) protein inhibitors; prostaglandins; percutaneous tibial nerve stimulation; 5-alpha reductase inhibitors; and phosphodiesterase-5 inhibitors. Further embodiments describe once-daily treatments, wherein the one or more additional therapeutic agents may be administered prior to, simultaneously with, or following the administration of the pharmaceutical composition comprising solabegron.

In one embodiment, the present application describes a once-daily treatment for LUTS, obesity, type 2 diabetes, heart failure, irritable bowel syndrome and similar gastrointestinal disorders, pre-term labor, depression and anxiety that achieves a desired blood plasma Cₘₐₓ while also not desensitizing the beta-3 adrenoceptor, comprising at least one immediate release composition, comprising solabegron and at least one pharmaceutically acceptable carrier or diluent; and at least one modified release composition, comprising solabegron and at least one pharmaceutically acceptable carrier or diluent. In some embodiments the pharmaceutical composition may have any of the Cₘₐₓ, Cₘᵢₙ, Tₘₐₓ, or Tₘᵢₙ described herein. Further embodiments describe once-daily treatments, wherein the at least one immediate release composition comprises about 75 mg to about 250 mg solabegron. Further embodiments describe once-daily treatments, wherein the at least one modified release composition comprises about 100 mg to about 400 mg solabegron. Further embodiments describe once-daily treatments, wherein the at least one immediate release composition achieves a blood plasma Cₘₐₓ in about 0.75 to about 4 hours after administration to a subject in need of treatment. Further embodiments describe once-daily treatments, wherein the at least one modified release composition achieves a blood plasma Cₘₐₓ in about 2 to about 8 hours after the first Cₘᵢₙ. Further embodiments describe once-daily treatments, further comprising administering one or more additional therapeutic agents or treatments useful for the treatment of LUTS, obesity, type 2 diabetes, heart failure, irritable bowel syndrome and similar gastrointestinal disorders, pre-term labor, depression and anxiety, wherein the one more additional therapeutic agents or treatments are selected from the groups consisting of: antimuscarinic agents; alpha adrenoceptor blockers; botulinum toxin; purinergics; cannabinoids; transient receptor potential (TRP) protein inhibitors; prostaglandins; percutaneous tibial nerve stimulation; 5-alpha reductase inhibitors; and phosphodiesterase-5 inhibitors. Further embodiments describe once-daily treatments, wherein the one or more additional therapeutic agents may be administered prior to, simultaneously with, or following the administration of the pharmaceutical composition comprising solabegron.

In one embodiment the present application describes a once-daily treatment for LUTS, obesity, type 2 diabetes, heart failure, irritable bowel syndrome and similar gastrointestinal disorders, pre-term labor, depression and anxiety that achieves a desired blood plasma Cₘₐₓ while also not desensitizing the beta-3 adrenoceptor or the biochemical pathways leading to the functional response, comprising a pharmaceutical composition, comprising a therapeutically effective amount of solabegron, wherein the pharmaceutical composition releases at least two releases of solabegron, wherein a first release of solabegron achieves a first target Cₘₐₓ, a second release of solabegron achieves a second target Cₘₐₓ, a first target Cₘᵢₙ is achieved between the first release and the second release and a second Cₘᵢₙ is achieved after the second release. Additional embodiments describe treatments, wherein said first target Cₘₐₓ is about 1.5 μg/mL to about 4 μg/mL, said second target Cₘₐₓ is about 1.5 μg.mL to about 4 μg/mL, said first Cₘᵢₙ is about 0.5 μg/mL to about 1.5 μg/mL and said second Cₘᵢₙ is about 0.01 μg/mL to about 0.5 μg/mL. Further embodiments describe treatments further comprising administering one or more additional therapeutic agents useful for the treatment of LUTS, obesity, type 2 diabetes, heart failure, irritable bowel syndrome and similar gastrointestinal disorders, pre-term labor, depression and anxiety, wherein the one more additional therapeutic agents are selected from the groups consisting of: antimuscarinic agents; alpha adrenoceptor blockers; 5-alpha reductase inhibitors; and phosphodiesterase-5 inhibitors, wherein the one or more additional therapeutic agents may be administered prior to, simultaneously with, or following the administration of the pharmaceutical composition comprising solabegron.

In one embodiment the present application describes a once-daily treatment for LUTS, obesity, type 2 diabetes, heart failure, irritable bowel syndrome and similar gastrointestinal disorders, pre-term labor, depression and anxiety that achieves a desired blood plasma Cₘₐₓ while also not desensitizing the beta-3 adrenoceptor, comprising at least one immediate release composition, comprising solabegron and at least one pharmaceutically acceptable carrier or diluent; and at least one modified release composition, comprising solabegron and at least one pharmaceutically acceptable carrier or diluent. In some embodiments the pharmaceutical composition may have any of the Cₘₐₓ, Cₘᵢₙ, Tₘₐₓ, or Tₘᵢₙ described herein. Additional embodiments describe treatments, wherein the at least one immediate release composition comprises about 100 mg to about 300 mg solabegron and the at least one modified release composition comprises about 100 mg to about 300 mg solabegron. Further embodiments describe treatments, wherein the at least one immediate release composition achieves a blood plasma Cₘₐₓ in about 1 to about 3 hours after administration to a subject in need of treatment and the at least one modified release composition achieves a blood plasma Cₘₐₓ in about 5 to about 11 hours after administration to a subject in need of treatment. Still further embodiments describe treatments, further comprising administering one or more additional therapeutic agents useful for the treatment of LUTS, obesity, type 2 diabetes, heart failure, irritable bowel syndrome and similar gastrointestinal disorders, pre-term labor, depression and anxiety, wherein the one more additional therapeutic agents are selected from the groups consisting of: antimuscarinic agents; alpha adrenoceptor blockers; 5-alpha reductase inhibitors; and phosphodiesterase-5 inhibitors, wherein the one or more additional therapeutic agents may be administered prior to, simultaneously with, or following the administration of the pharmaceutical composition comprising solabegron.

### EXAMPLES

HEK cells transfected with the human beta-3 adrenoceptor according to the method of Vrygag et al (2009) will be employed. Additional cell lines, such as CHO, SK-N-MC neuroblastoma cells or cultured human adipocytes may be considered.

For the desensitization experiments, the cells will be cultured for 0.5 hr to 24 hr in a serum-free medium in the presence of vehicle or a concentration of 0.01 to 10-μM beta-3 adrenoceptor agonists. Beta-3 agonists that may be studied include solabegron, CL 316,243 or isoproterenol. Cells will be washed with serum-free medium for a period of 1 to 4 hr.

### Example 1

### Cyclic AMP Accumulation or ERK Activation Endpoint Assessment of Beta-3 Adrenoceptor Desensitization

Cells will be detached from the surface using enzyme-free cell dissociation buffer and washed once with Hank's balanced salt solution (HBSS). Cells will be re-suspended in HBSS supplemented with 5 mM HEPES and 0.05% bovine serum albumin. The cells will be stimulated with the appropriate concentration-response to a beta-3 adrenoceptor agonist or vehicle. The stimulation mixture will contain the cAMP phosphodiesterase inhibitors IBMX and RO 20-1724 (100 μM each). Cells will be added to the stimulation mixture 1:1 in a 384 well optiplate and stimulated for 30 min at room temperature. cAMP detection will be using a LANCE® cAMP Kit (PerkinElmer). ERK activation will be measured by ELISA. Desensitization at the level of adenylyl cyclase will be confirmed by measuring the response to forkolin.

### Example 2

### Radioligand-Binding Studies Endpoint Assessment of Beta-3 Adrenoceptor Desensitization

[³H]-L 748,337 saturation radioligand binding will be performed as previously described (van Wieringen et al. 2011). Briefly, cells at approximately 80% confluence will be washed with PBS, harvested by scraping the culture flasks with a cell scraper, washed twice by centrifugation, and then homogenized in ice-cold buffer (50 mM Tris, 0.5 mM EDTA, pH 7.5). The homogenates will be centrifuged for 20 min at 50,000×g at 4° C. The pellets will be resuspended in buffer and stored at −80° C. Aliquots of the respective membrane preparation (approximately 50-100 μg protein/assay) will be incubated in a total volume of 250 μl of binding buffer (10 mM Tris, 0.9 mN NaCl at pH 7.4) at 25° C. for 60 min. Non-specific binding will be defined using 100 μM isoproterenol. In saturation experiments, eight radioligand concentrations will be used. All experiments will be performed in duplicates in 96 well plates, and incubations will be terminated by rapid vacuum filtration. Each filter will be washed with approximately 2-3 mL of ice-cold buffer. Radioactivity adherent to the filters will be quantified in Perkin Elmer scintillator counter.

### Example 3

### Alteration in G-Protein Expression Endpoint Assessment of Beta-3 Receptor Desensitization

Cells treated with beta-3 agonists at various time-points will be washed with PBS, harvested, homogenized and centrifuged. The pellets will be re-homogenized, boiled loaded on to SDS gels and electrophoresed for approximately 1 hr at 40 mA. Primary antibodies (rabbit polyclonal) for detection of G protein subunits (G_{S}, Gi1,Gi2, Gi3,Gq/11) will be used. Immunoblotting will be performed for approximately 12 hr at 4° C. Following washing, a secondary antibody (i.e. donkey anti-rabbit coupled to horseradish peroxidase) will be used. Luminescence signals will be detected and quantified.

### Example 4

### Prevention of Receptor Desensitization

Macroscopically normal bladders obtained from Male CD rats (220-250 g) were used. Tissues were rejected if they did not respond adequately to viability checks. Each bladder was cleaned free of surrounding connective tissue and halved longitudinally. The bladder longitudinal smooth muscle, with the urothelium still attached were mounted in 25 mL organ baths containing physiological saline solution (PSS) (composition: 119.0 mM NaCl, 4.7 mM KCl, 1.2 mM MgSO₄, 24.9 mM NaHCO₃, 1.2 mM KH₂PO₄, 2.5 mM CaCl₂ and 11.1 mM glucose) supplemented with 1 μM prazosin (α₁-adrenoceptor antagonist) and 30 nM ICI118551 (β₂-adrenoceptor antagonist), aerated with 95% O₂/5% CO₂ gas mix, warmed and maintained at approximately 37° C. throughout the experiment. Changes in force production were recorded using transducers. After mounting, the bladder halves were allowed to equilibrate for at least 30 min before they were set to a stable tension of approximately 1.0 g. The tissue was then allowed to equilibrate over at least a 60 min period with washes every 15 min.

EFS parameters and viability check

In initial experiments the parameters for the EFS were assessed by performing a frequency curve to determine a frequency that would give a response that was approximately 80% of the response seen to 80 mM KCl. Optimal EFS parameters were determined to be: 30 Volts, square pulse of 0.1 ms, train of 4 seconds every 120 seconds, 15 Hz. This frequency was then used to stimulate the tissue for all subsequent experiments. The viability of bladder strips was tested by stimulating the tissue with EFS for minimum of ten minutes. Tissues that failed to produce a response of at least 1.0 g were rejected.

### Experimental Protocol

Pilot studies to determine EC₉₀ concentrations of the test compounds were performed. Upon stabilization of the baseline tension, the bladder muscle strips were stimulated with EFS parameters described above. The resulting contractile responses were allowed to stabilize before adding cumulative concentrations of test compound (half-log increments) in a cumulative concentration response curve (CCRC). A vehicle and a positive control (CL-316,243) were run in the same manner in order to compare with the test compounds. From the data obtained in these experiments, EC₉₀ concentrations of the test compounds were determined for use in subsequent experiments.

The bladder muscle strips were incubated with the EC₉₀ determined for each test compound in the pilot studies, for a period of 1 or 3 hr. Following compound incubation, the tissues were washed with PSS for a period of 1, 3 or 6 hr, with washes approximately every 15 min, to remove the drug. At the end of the final wash period the tissues were stimulated with EFS, and left to equilibrate for at least 30 min. A CCRC was then performed in each tissue. Tissue responses were calculated as the mean (SEM) and expressed as percentage of EFS induced tone.

Determination of EC₉₀ concentration for beta-3 adrenoceptor agonists to inhibit EFS-induced contraction in rat isolated bladder

In the initial pilot experiments, concentration response curves were performed to the test compounds to determine an EC₉₀ value for use in later experiments. The calculated values for CL-316,243 and solabegron were 0.042 and 1.0 μM, respectively.

Effect of the Washout Period Following a One Hr Incubation with Test Compound

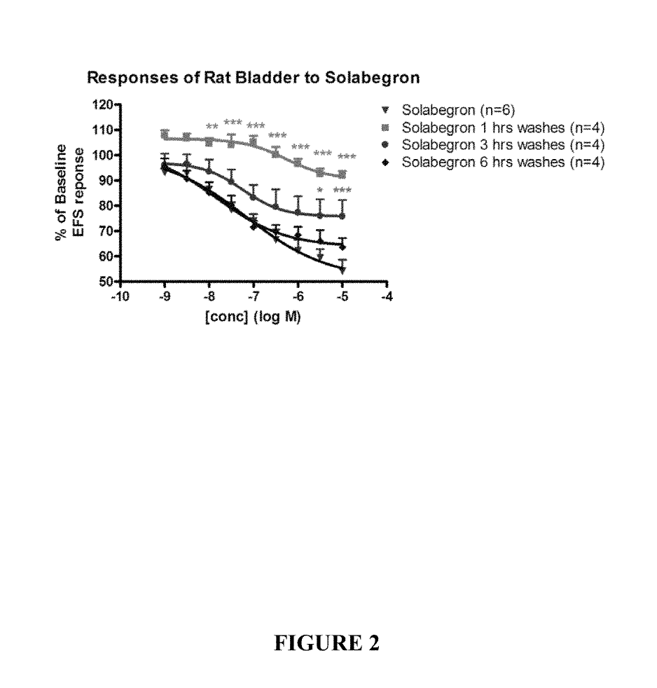

Tissues were incubated with the EC₉₀ concentration of the test compounds for one hr followed by one hr, three hr or six hr of washing with PSS. After only one hr of washing, the response to solabegron was significantly attenuated (FIG. 2). Responses to higher concentrations of solabegron were also significantly attenuated after three hr of washing. After six hr of washing the response to solabegron was similar to that seen in tissues that had not been pre-exposed to the test compound (FIG. 2). This EFS-induced potentiation of bladder contraction from baseline was not observed with solabegron. These data suggest that incubation of the rat bladder with beta-3 adrenoceptor agonists produced marked receptor desensitization, and that the receptor is re-sensitized in a time-dependent manner following removal of the agonist by removing the ligand by washing out. Effect of the washout period following a three hr incubation with test compound.

In the next series of experiments, tissues were incubated with the EC₉₀ concentration of the test compounds for three hr followed by either one hr, three hr or six hr of washing with PSS.

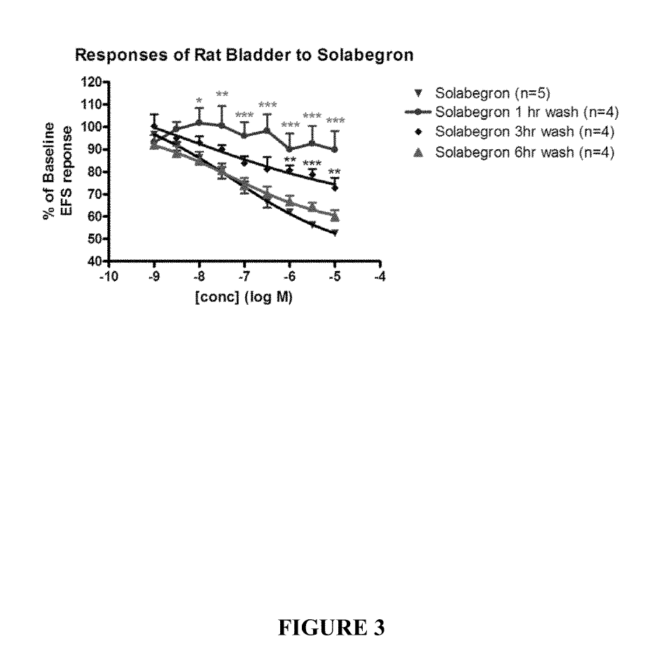

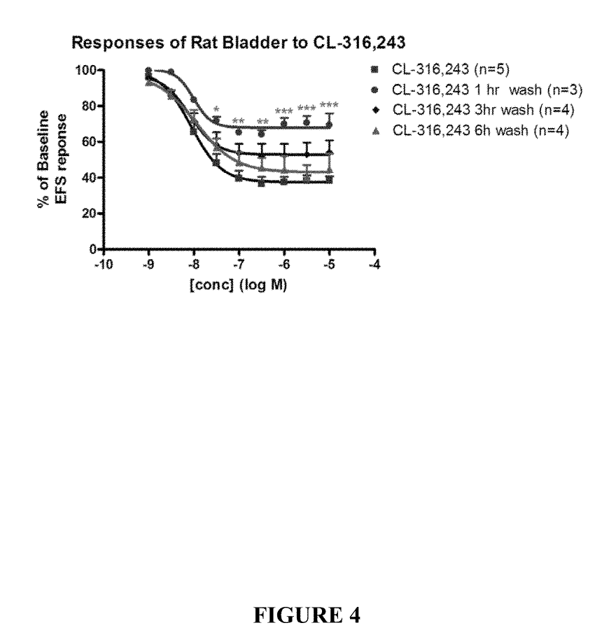

After only one hr of washing, the responses to solabegron and CL-316,243 were significantly attenuated (FIGS. 3 AND 4). Responses to the highest concentrations of solabegron were also significantly attenuated after three hr of washing. After six hr of washing the responses to solabegron or the tool compound CL-316,243 were similar to that seen in tissues which had not been pre-exposed to the test compound (FIGS. 3 AND 4).

### Conclusions:

The data in the present experiments demonstrate that prolonged administration of beta-3 adrenoceptor agonists produce time-dependent desensitization of the beta-3 adrenoceptor in the rat bladder. Recovery of receptor desensitization was achieved by removal or washing-out the agonist from the tissue, such that the receptor-mediated functional response in the bladder returns to vehicle-treated or baseline conditions.

Following either a one hr or three hr incubation with the EC₉₀ concentration of solabegron, the ability of solabegron to reduce the magnitude of EFS-mediated responses in rat bladder muscle was still attenuated markedly when the tissue was washed for only one or three hr. After 6 hr of washing post-incubation, the ability of solabegron to reduce EFS was not significantly different than that seen in muscle strips that had only been exposed to an equivalent volume of vehicle and not to solabegron. These data indicate that the effects of the exposure of the tissue to an EC₉₀ concentration of solabegron produced desensitization of the responses mediated by the beta-3 adrenoceptor. The EC₉₀ concentration of the beta-3 adrenoceptor agonists used in this study was selected because it reflects a clinically relevant concentration comparable to the Cₘₐₓ observed in subjects. Beta-3 receptor desensitization appeared to occur rapidly, as only 1 hr incubation was necessary to produce marked inhibition of the beta-3 receptor mediated response.

The re-sensitization response occurred in a time-dependent manner, indicating the functional defect in the tissue was reversible, and recovery was completed within 6 hr. Such a time course of desensitization and re-sensitization is consistent with the time course that will be used for a pulsatile formulation administration of solabegron in subjects.

CL-316,243 was used as a reference standard as a rodent selective beta-3 adrenoceptor agonist. Attenuation in the ability of CL-316,243 to reduce the magnitude of EFS responses in rat bladder muscle tissue was also seen after a three hr pre-incubation to the EC₉₀ concentration of CL-316,243. Following washout of CL-316,243 the recovery of the beta-3 adrenoceptor mediated response occurred in a time-dependent manner, as was seen with solabegron.

The data in the present experiments demonstrate that prolonged administration of beta-3 adrenoceptor agonists can produce time-dependent desensitization of the beta-3 adrenoceptor-mediated responses in the rat bladder. Recovery of receptor desensitization and prevention of prolonged receptor desensitization can be achieved by removal of the agonist from the tissue, such that the receptor-mediated functional response returns to baseline conditions. Beta-3 receptor desensitization can be prevented by giving sufficient time between drug exposures for the tissue to recover. Therefore, prevention of prolonged administration of a beta-3 adrenoceptor agonist in subjects with OAB may be desirable in order to preserve and increase therapeutic efficacy. Thus, the daily administration of a beta-3 adrenoceptor agonist that is formulated to occur in a pulsatile manner may be the viable approach for chronic treatment. Such an approach will reduce beta-3 adrenoceptor desensitization and promote recovery of desensitized receptors to become active.

### Example 5

### Prevention of Receptor Desensitization in Human Bladder

Beta-3 adrenoceptor desensitization and resensitization were examined in the human bladder. Similar to the protocol used in the rat bladder, isolated human bladder tissue strips were studied for EFS responses. Optimal EFS parameters were determined to be: 30 volts, square pulse of 0.1 ms, train of 5 seconds every 60 seconds, 8-10 Hz. Solabegron (10-10000 μM) produced concentration-dependent inhibition of EFS-induced bladder smooth muscle contraction. Human bladder muscle strips were incubated with the EC₉₀ for solabegron for a period of 1 or 3 hr. Following compound incubation, the tissues were washed with PSS for a period of 1 or 3 hr, with washes approximately every 15 min, to remove the drug. At the end of the final wash period the tissues were stimulated with EFS, and left to equilibrate for at least 30 min. A concentration-response curve was then performed in each tissue.

Similar to the data obtained in the rat bladder, the data in these experiments demonstrate that prolonged and sustained administration of a beta-3 adrenoceptor agonist produce time-dependent desensitization of the beta-3 adrenoceptor in the human bladder. Recovery of receptor desensitization was achieved by removal or washing-out the agonist from the tissue, such that the receptor-mediated functional response in the bladder returns to vehicle-treated or baseline conditions. The re-sensitization response occurred in a time-dependent manner, indicating the functional defect in the tissue was reversible, and recovery was time-dependent. Such a time course of desensitization and re-sensitization is consistent with the time course that will be used for a pulsatile formulation administration of solabegron in subjects.

### Example 6

### Multiparticulate Formulation for the Release of Solabegron

A formulation utilizing solabegron is proposed, wherein pellets or mini-tablets containing solabegron will form the basis for multiple releases of solabegron to a subject in need. The formulation will contain at least two populations of pellets, wherein at least one population comprises an immediate or early release population and at least one population comprises a modified (i.e. sustained and/or delayed) release population. The immediate release pellets will release solabegron immediately or soon thereafter in the GI tract, whereas the modified release pellets will release solabegron at a later time or distance inside the GI tract. The modified release pellets may be coated with either a pH dependent (enteric) coating or a time dependent coating so as to delay the second release of solabegron to the desired position in the GI tract. Both types of pellets may contain any physical form of solabegron such as, for example, amorphous or crystalline solid. The pellets may be drug-layered pellets, matrix-type pellets, or mini-tablets containing active drug in its matrix.

### Example 7

### Drug Coated Spheres/Pellet with an Inert Core for the Release of Solabegron

A formulation utilizing solabegron is proposed, wherein spheres/pellets with an inert core layered with solabegron will form the basis for multiple releases of solabegron to a subject in need. The formulation will contain at least two populations of spheres/pellets with an inert core, wherein at least one population comprises an immediate or early release population and at least one population comprises a modified (i.e. sustained and/or delayed) release population. The immediate, early spheres/pellets with an inert core will release solabegron immediately or soon thereafter in the GI tract, wherein the modified release spheres/pellets with an inert core will release solabegron at a later time inside the GI tract. The modified release spheres/pellets with an inert core may be coated with either a pH dependent (enteric) coating or a time dependent coating so as to delay the second release of solabegron to the desired position in the GI tract. Both types of spheres/pellets with an inert core may contain any physical form of solabegron such as, for example, amorphous or crystalline solid.

### Example 8

### Multi-Layer Tablet for the Release of Solabegron

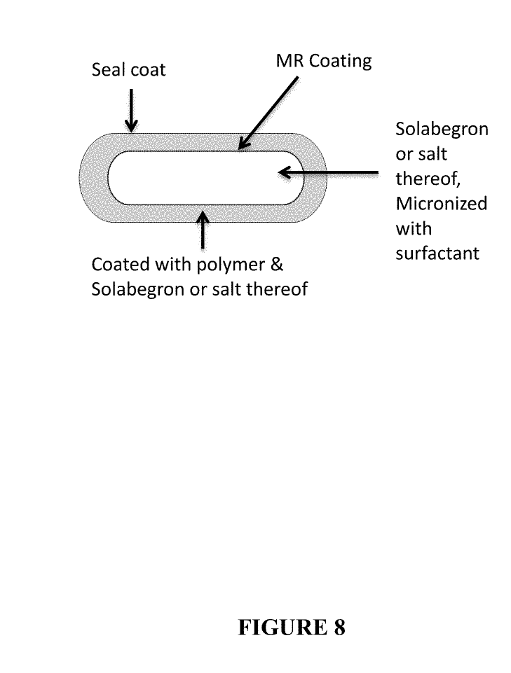

A formulation utilizing solabegron is proposed, wherein a multi-layer tablet containing solabegron will form the basis for the multiple releases of solabegron to a subject in need. The formulation will contain a tablet having both an immediate/early release layer and a modified release layer. The immediate layer will release solabegron immediately or soon thereafter in the GI tract, whereas the modified release layer will release solabegron at a later time inside the GI tract. The modified release layer may be coated with either a pH dependent (enteric) coating or a time dependent coating so as to delay the second release of solabegron to the desired position in the GI tract. The layers may contain any physical form of solabegron such as, for example, amorphous or crystalline solid. An example of a multi-layer tablet is shown in FIG. 8.

### Example 9

### Matrix Tablet for the Release of Solabegron

A formulation utilizing solabegron is proposed, wherein a matrix tablet containing solabegron will form the basis for the multiple releases of solabegron to a subject in need thereof. The formulation will contain a well-mixed composite of drug(s) with rate-controlling excipients. Numerous sustained and/or delayed release tablets such as membrane controlled system, matrices with water soluble/insoluble polymers, and osmotic systems may be utilized. The tablet may contain either the amorphous form of solabegron or the crystalline form. The delayed/sustained release can be achieved by applying a permeable or semipermeable membrane to the tablet core or by mixing the drug with excipient that is either a hydrophilic polymer with high viscosity and gel forming capability or a hydrophobic excipient that slows down the diffusion of drug molecule. An immediate release drug layer can be coated or press coated to the tablet that will be available for an early release in the GI tract, while the delayed/sustained release core will be designed to delay the drug release after a time period in a designed region of the GI tract.

### Example 10

### Multicore Tablet or Capsule for the Release of Solabegron

A formulation utilizing solabegron is proposed, wherein a multicore tablet or capsule containing solabegron will form the basis for the multiple releases of solabegron to a subject in need thereof. The formulation will contain a multicore tablet or capsule that comprises multiple discrete cores consisting of at least one immediate release core and at least one delayed/sustained release core contained within the same tablet or capsule. The at least one immediate release core will be available for an early release in the GI tract, while the at least one delayed/sustained release core will be designed to delay the drug release after a time period in a designed region of the GI tract.

### Example 11

### Gastroretentive Delivery System for the Release of Solabegron

A formulation utilizing solabegron is proposed, wherein a gastroretentive oral dosage form containing solabegron will form the basis for the multiple releases of solabegron to a subject in need. The formulation will contain a tablet or capsule having both an immediate release and modified release component. The immediate layer will release solabegron immediately in the GI tract, wherein the modified release layer will release solabegron at a later time inside the GI tract. The gastroretentive oral dosage form may utilize mucoadhesive, swellable, high density or floating technologies to prolong residence time in the stomach thereby allowing a prolonged period for release of both first and second releases in the stomach or upper GI. Both releases may contain any physical form of solabegron such as, for example, amorphous or crystalline solid.

### Example 12

### Proposed Formulations for Pellets in Solabegron Formulations

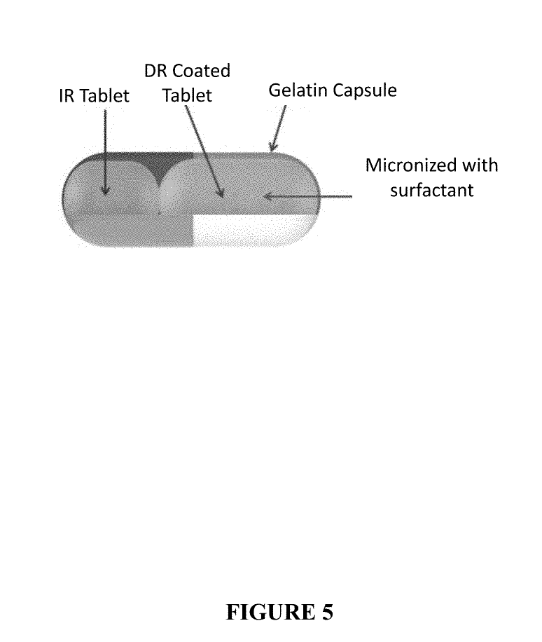

TABLE 1 illustrates the dosage formulations comprising amorphous solabegron and a polymer with or without excipients formed into small 2 mm diameter tablets by either hot-melt extrusion (HME) or spray drying techniques. The drug loading of solabegron to polymer may be in any ratio from 1:99 to 99:1 by weight. The final dosage form includes a proportion of tablets that are immediate release formulation and a proportion of tablets that are modified release formulation that may receive additional coating enclosed in a capsule as illustrated in FIG. 5. The tablets that are additionally coated, receive a coating that prevents dissolution in the GI until they reach the appropriate location in the GI. At which point, the coating dissolves and breaks away from the tablet. There may optionally be a third population of tablets having an immediate release core but coated with a non-pH dependent polymer. The non-pH dependent coating may then be further coated so as to allow dissolution at a chosen point in the GI.

TABLE 1

| | | | Plasticizer |
|---|---|---|---|
| Processing | | Drug | of other |
| Method | Polymer | Load | excipients |
| HME | HPMC | .1 to .5 | Yes or No |
| HME | Polyethylene Glycol MW = 20000 | .1 to .5 | Yes or No |
| HME | Hydroxypropylmethyl cellulose acetate | .1 to .5 | Yes or No |
| | succinate | | |
| HME | Hydroxypropylmethyl cellulose | .1 to .5 | Yes or No |
| | phthalate (HPMCP) | | |
| HME | Hydroxypropyl β Cyclodextrin | .1 to .5 | Yes or No |
| HME | PVP K30 | .1 to .5 | Yes or No |
| HME | Hydroxypropyl cellulose | .1 to .5 | Yes or No |
| HME | Poloxamer 188 | .1 to .5 | Yes or No |
| HME | Poloxamer 407 | .1 to .5 | Yes or No |
| Spray Dry | HPMC | .1 to .5 | Yes or No |
| Spray Dry | Polyethylene Glycol MW = 20000 | .1 to .5 | Yes or No |
| Spray Dry | Hydroxypropylmethyl cellulose acetate | .1 to .5 | Yes or No |
| | succinate | | |
| Spray Dry | Hydroxypropylmethyl cellulose | .1 to .5 | Yes or No |
| | phthalate (HPMCP) | | |
| Spray Dry | Hydroxypropyl β Cyclodextrin | .1 to .5 | Yes or No |
| Spray Dry | PVP K30 | .1 to .5 | Yes or No |
| Spray Dry | Hydroxypropyl cellulose | .1 to .5 | Yes or No |
| Spray Dry | Poloxamer 188 | .1 to .5 | Yes or No |
| Spray Dry | Poloxamer 407 | .1 to .5 | Yes or No |

### Example 13

### Proposed Formulations for the Release of Solabegron

It is proposed that solabegron be formulated as a once-daily formulation having two distinct release components. It is envisioned that such formulations may exist wherein both the two release components contain the same or different amounts of solabegron and when different either the first or second release may contain the greater amount of solabegron. Provided below in TABLE 2 are formulations that should provide a therapeutic amount of solabegron to a subject in need without desensitizing the beta-3 adrenoceptor.

TABLE 2

| | Low Dose | High Dose | Mixed Dose |
|---|---|---|---|
| Cₘₐₓ 1 (hours after | 0.75-4.0 | 0.75-4.0 | 0.75-4.0 |
| administration) | | | |
| Cₘᵢₙ 1 (hours after | 4.0-8.0 | 4.0-8.0 | 4.0-8.0 |
| administration) | | | |
| Cₘₐₓ 2 (hours after | 2.0-8.0 | 2.0-8.0 | 2.0-8.0 |
| Cₘᵢₙ 1) | | | |
| Cₘᵢₙ 2 (hours after | 24 | 24 | 24 |
| administration) | | | |
| Time Between Cₘₐₓ 1 | 2.0-8.0 | 2.0-8.0 | 2.0-8.0 |
| and Cₘₐₓ 2 (hours) | | | |
| Cₘₐₓ 1 (μg/mL) | 0.5-2 | 1.5-3.5 | 0.5-2 |
| Cₘₐₓ 2 (μg/mL) | 1.5-3 | 2.5-4 | 2.5-4 |
| Cₘᵢₙ 1 (μg/mL) | 0.25-1.0 | 0.5-1.5 | 0.25-1.0 |
| Cₘᵢₙ 2 (μg/mL) | 0.01-1 | 0.25-1 | 0.01-1 |
| First Release | 75-250 | 75-250 | 75-250 |
| Second Release | 100-400 | 100-400 | 100-400 |
| Time at or below | about 6 to | about 6 to | about 6 to |
| 1.0 μg/mL over a | about 9 | about 9 | about 9 |
| 24 hour period | | | |
| (hours) | | | |

### Example 14

### Dissolution of Solabegron Formulations of Example 12

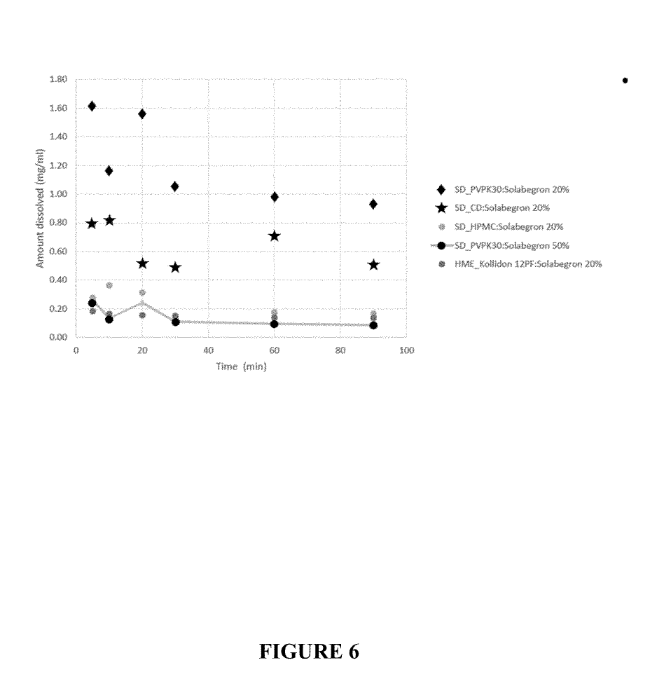

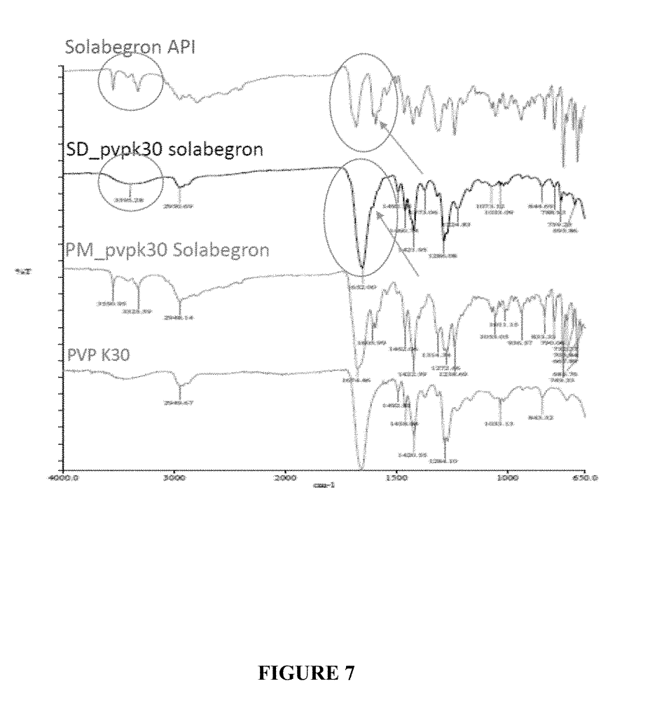

Certain solabegron formulation described in Example 12 were subjected a dissolution study, wherein the solabegron-polymer formulation was placed in a phosphate-buffered saline (PBS) solution at pH 6.8. The amount of solabegron in solution is illustrated as a function of time in FIG. 6. A formulation of 20% weight solabegron spray-dried in polyvinyl pyrrolidine (PVPK30) demonstrated good dissolution properties and was found to form a uniform amorphous phase by Fourier-transform infrared spectroscopy (FTIR) FIG. 7.

### Example 15

### Stability of Solabegron-Polymer Pellets

A series of formulations comprising amorphous solabegron and a polymer formed into small 2 mm diameter tablets by either hot-melt extrusion (HME) or spray drying techniques were prepared according to TABLE 3. The actual loading weight percentage of solabegron was determined at formation and the purity of the product was measured after one week to determine the amount of product degradation.

TABLE 3

| | | Solabegron(Salt) | Purity |
|---|---|---|---|
| Formulation | Stability (Temp/Days) | Loading(%) | (% Area) |
| HME_Kollidon 12PF:Solabegron (80:20) | 40 C./75RH Closed, 8 days | 16.6 | 98.95 |
| HME_Kollidon 12PF:Solabegron (80:20) | 40 C./75RH Open, 8 days | 15.2 | 98.56 |
| HME_Kollidon 12PF:Solabegron (80:20) | RT, 8 days | 16.7 | 98.99 |
| SD_PVP K30:Solabegron (80:20) | 40 C./75RH Closed, 8 days | 17.8 | 99.82 |
| SD_PVP K30:Solabegron (80:20) | 40 C./75RH Open, 8 days | 16.9 | 99.90 |
| SD_PVP K30:Solabegron (80:20) | RT, 8 days | 17.9 | 100 |
| SD_CD:Solabegron (80:20) | 40 C./75RH Closed, 7 days | 19.5 | 100 |
| SD_CD:Solabegron (80:20) | 40 C./75RH Open, 7 days | 18.0 | 98.32 |
| SD_CD:Solabegron (80:20) | RT, 7 days | 19.3 | 100 |
| SD_HPMC:Solabegron (80:20) | 40 C./75RH Closed, 7 days | 22.0 | 100 |
| SD_HPMC:Solabegron (80:20) | 40 C./75RH Open, 7 days | 21.4 | 98.82 |
| SD_HPMC:Solabegron (80:20) | RT, 7 days | 21.8 | 99.83 |
| PVP K30:Solabegron (50:50) | Initial(T0) | 49.1 | 100 |

### Example 16

### Spheronized Compositions of Solabegron

A formulation utilizing solabegron is proposed, wherein solabegron is made into small spheres or spheroids. The formulation will contain at least two populations of spheres, wherein at least one population comprises an immediate or early release population and at least one population comprises a modified (i.e. sustained and/or delayed) release population. The immediate, early spheres release solabegron immediately or soon thereafter in the GI tract, wherein the modified release spheres will release solabegron at a later time inside the GI tract. The modified release spheres may be coated with either a pH dependent (enteric) coating or a time dependent coating so as to delay the second release of solabegron to the desired position in the GI tract. Both types of spheres may contain any physical form of solabegron such as, for example, amorphous or crystalline solid.

### Example 17

### Composition A: 200 mg Solabegron Modified Release Tablet with Sodium Lauryl Sulfate (SLS)

A pressed tablet core containing solabegron HCl freebase equivalent and additional excipients was prepared. The core is sub-coated with Opadry Clear 03019001 followed by a final coating of PlasACRYL HTP20/Eudragit L30 D-55. The final Eudragit coating ensures release at approximately pH 5.5 and greater. The composition is more specifically defined as follows:

| | Chemical Name | % w/w |
|---|---|---|
| Tablet | Micronized Solabegron HCl (D90 = ~4 um) | 27.5 |
| Core | Mannitol | 37.9 |
| | Microcrystalline Cellulose | 9.2 |
| | Croscarmellose Sodium | 9.1 |
| | Citric Acid | 2.8 |
| | Sodium Lauryl Sulfate | 2.8 |
| | Colloidal SiO2 | 0.5 |
| | Sodium Stearyl Fumarate | 1.9 |
| | Sub-total | 91.6 |
| DR | Opadry Clear | 2.7 |
| Layer | Eudragit L30 D55 | 4.5 |
| | Plasacryl HTP20 | 1.1 |
| | Total | 100 |

### Example 18

### Composition B: 200 mg Solabegron Modified Release Tablet with Poloxamer 188 (P188)

A pressed tablet core containing solabegron HCl freebase equivalent and additional excipients was prepared. The core is sub-coated with Opadry Clear 03019001 followed by a final coating of PlasACRYL HTP20/Eudragit L30 D-55. The final Eudragit coating ensures release at approximately pH 5.5 and greater. The composition is more specifically defined as follows:

| | Chemical Name | % w/w |
|---|---|---|
| Tablet | Micronized Solabegron HCl (D90 = ~4 um) | 27.5 |
| Core | Mannitol | 31.6 |
| | Microcrystalline Cellulose | 9.2 |
| | Kolliphor P188 (aka Poloxamer 188) | 9.1 |
| | Croscarmellose Sodium | 9.1 |
| | Citric Acid | 2.8 |
| | Colloidal SiO2 | 0.5 |
| | Sodium Stearyl Fumarate | 1.9 |
| | Sub-total | 91.6 |
| DR | Opadry Clear | 2.7 |
| Layer | Eudragit L30 D55 | 4.5 |
| | Plasacryl HTP20 | 1.1 |
| | Total | 100 |

### Example 19

### Composition C: 200 mg Solabegron Modified Release Tablet with Sodium Lauryl Sulfate (SLS) and Poloxamer 188 (P188)

A pressed tablet core containing solabegron HCl freebase equivalent and additional excipients was prepared. The core is sub-coated with Opadry Clear 03019001 followed by a final coating of PlasACRYL HTP20/Eudragit L30 D-55. The final Eudragit coating ensures release at approximately pH 5.5 and greater. The composition is more specifically defined as follows:

| | Chemical Name | % w/w |
|---|---|---|
| Tablet | Micronized Solabegron HCl (D90 = ~4 um) | 27.5 |
| Core | Mannitol | 28.8 |
| | Microcrystalline Cellulose | 9.2 |
| | Kolliphor P188 (aka Poloxamer 188) | 9.2 |
| | Croscarmellose Sodium | 9.2 |
| | Citric Acid | 2.7 |
| | Sodium Lauryl Sulfate | 2.7 |
| | Colloidal SiO2 | 0.5 |
| | Sodium Stearyl Fumarate | 1.8 |
| | Sub-total | 91.6 |
| DR | Opadry Clear | 2.7 |
| Layer | Eudragit L30 D55 | 4.5 |
| | Plasacryl HTP20 | 1.1 |
| | Sub-total | 100 |

### Example 20

### Composition D: 275 mg Solabegron Combined Immediate/Early Release and Modified Release Tablet with Sodium Lauryl Sulfate (SLS)

A pressed tablet core containing solabegron HCl freebase equivalent and additional excipients was prepared. The core is sub-coated with Opadry Clear 03019001 followed by a final coating of PlasACRYL HTP20/Eudragit L30 D-55. The final Eudragit coating ensures release at approximately pH 5.5 and greater. The Eudragit coated core is then coated with a 50/50 suspension of micronized solabegron HCl/Opadry Clear 03019001 until 75 mg of solabegron HCl freebase is applied. The composition is more specifically defined as follows:

| | Chemical Name | % w/w |
|---|---|---|
| Tablet | Micronized Solabegron HCl (D90 = ~4 um) | 22.8 |
| Core | Mannitol | 31.4 |
| | Microcrystalline Cellulose | 7.6 |
| | Croscarmellose Sodium | 7.5 |
| | Citric Acid | 2.3 |
| | Sodium Lauryl Sulfate | 2.3 |
| | Colloidal SiO2 | 0.4 |
| | Sodium Stearyl Fumarate | 1.6 |
| | Sub-total | 76 |
| DR | Opadry Clear | 2.3 |
| Layer | Eudragit L30 D55 | 3.8 |
| | Plasacryl HTP20 | 0.9 |
| | Sub-total | 82.9 |
| IR | Solabegron HCl | 8.5 |
| Layer | Opadry Clear | 8.5 |
| | Total | 100 |

### Example 21

### Modified Release Dosage Form Containing 30% Solabegron as a Suspension in an Enteric Capsule (100 mg Solabegron)

Micronized solabegron HCl suspension blended into a suspension with Tween 80 and Propylene Glycol. The suspension is weighed and dispensed into an enteric capsule designed to dissolve at approximately pH 5.5 and greater. The composition is more specifically defined as follows:

| Chemical Name | % w/w |
|---|---|
| Propylene Glycol | 33.65% |
| Tween 80 | 33.65% |
| Micronized Solabegron HCl (D90 = ~4 um) | 32.70% |
| Total | 100.00% |

### Example 22

### Fasted State Simulated Gastric Fluid (FaSSGF) Dissolution Studies of Solabegron Formulations

Dissolution studies were conducted under the following conditions: FaSSGF media, pH 1.6, 75rpm for the first 60 minutes, then 200 rpm until the infinity pull (120 minutes)
USP II paddles, 37.0 C.
500 mL media/vessel
45 um filters then 5.0 um filters, both collected and analyzed.
Sample pull at 10, 30, 60 minutes, then increase rpm and pull infinity at 120 minutes.

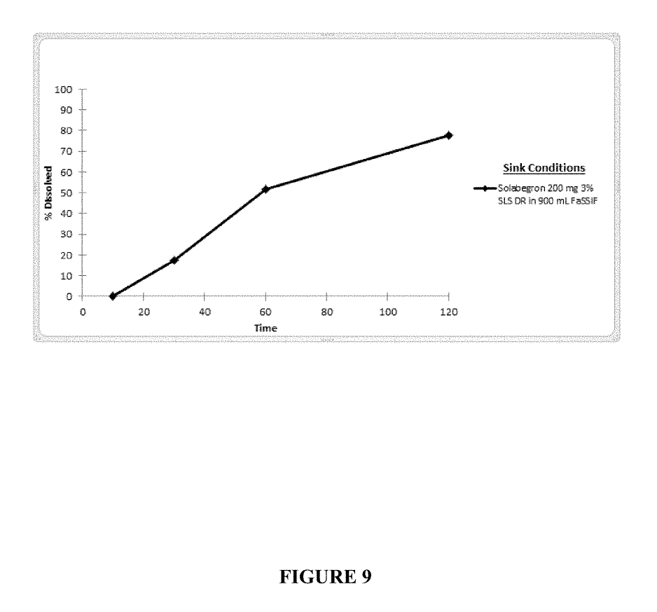

FIG. 9 shows the dissolution curve of composition A (Example 17).

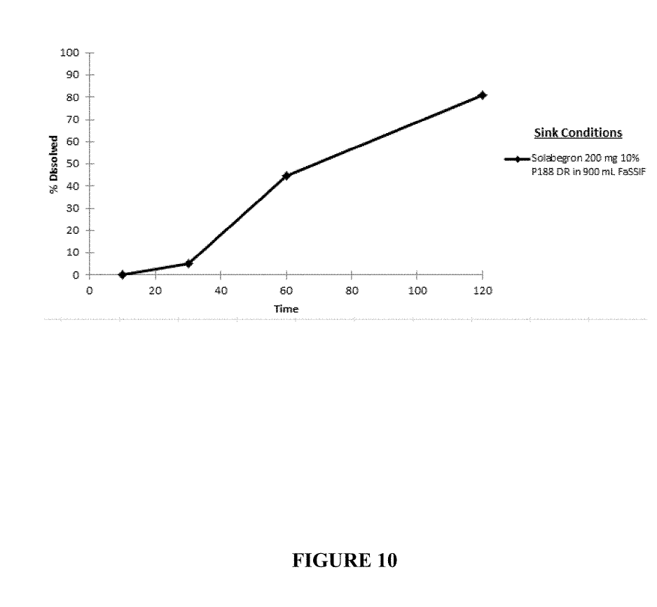

FIG. 10 shows the dissolution curve of composition B (Example 18).

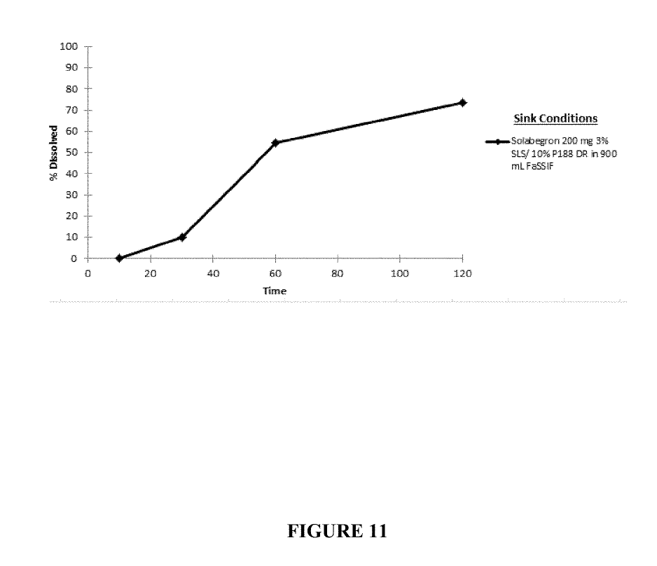

FIG. 11 shows the dissolution curve of composition C (Example 19).

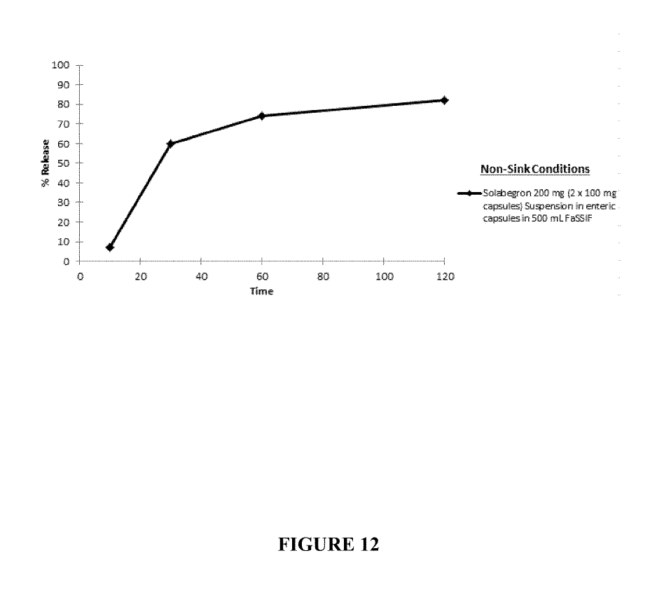

FIG. 12 shows the dissolution curve of composition E (Example 21).

Another dissolution study was conducted under the following conditions: FaSSGF media, pH 1.6, 75 rpm for the first 60 minutes, then 200 rpm until the infinity pull (120 minutes)
USP II paddles, 37.0 C.
500 mL media/vessel
45 um filters then 5.0 um filters, both collected and analyzed.
Sample pull at 10, 30, 60 minutes, then increase rpm and pull infinity at 120 minutes.

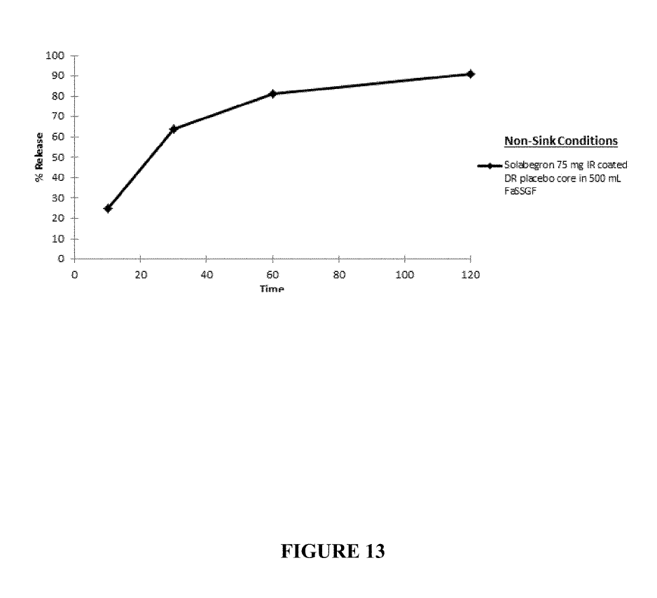

FIG. 13 shows the dissolution curve of a 75 mg immediate release coated delayed release placebo core in 500 mL of FaSSGF.

### Example 23

### Dissolution Studies of Solabegron Formulations

Dissolution studies were conducted under the following conditions:

| Condition | Setting | |
|---|---|---|
| Apparatus | USP II, Paddies | |
| | Tablets in Sinkers | |
| Agitation Rate | 75 RPM | |
| Vessel Temperature | 37° C. ± 0.5° C. | |
| Sample Volume | 5 mL (manual) | |
| Filters | 10 μm in-line | |
| Acid Stage Bath | Media Volume: | 1000 mL |
| 0.01N HCl | Sample Times: | 60, 120 minutes |
| | Sample Dilution: | N/A |
| Buffer Stage Bath | Media Volume: | 1000 mL |
| 0.05M Phosphate | Sample Times: | 30, 60, 120 minutes |
| Buffer, pH 6.8 | | (after media change) |
| with 2% SLS | Sample Dilution: | 2.5 mL sample into 2.5 |
| | | mL diluent |

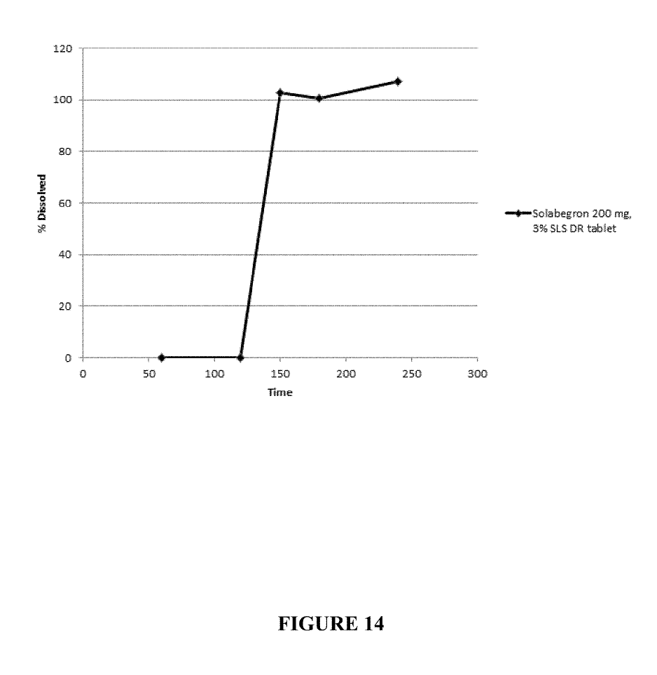

FIG. 14 shows the dissolution curve of composition A (Example 17).

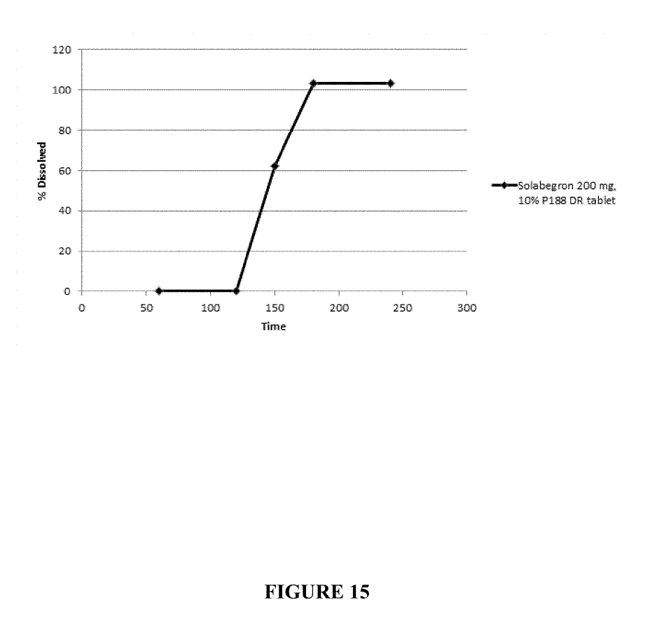

FIG. 15 shows the dissolution curve of composition B (Example 18).

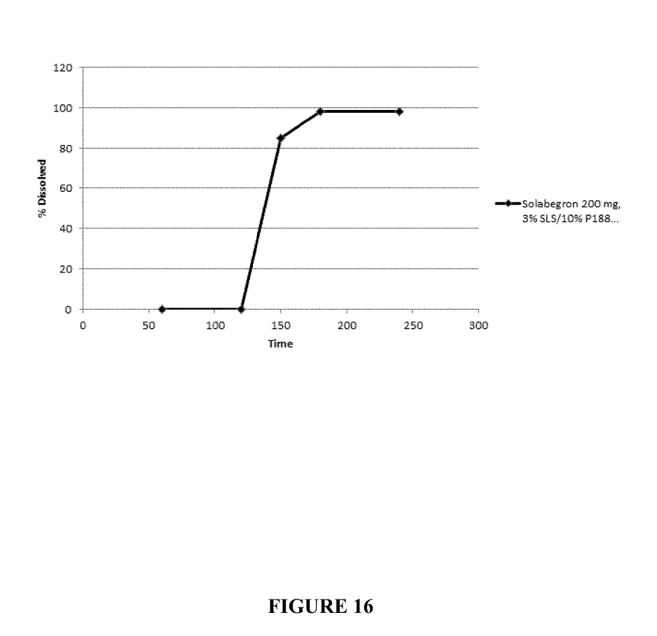

FIG. 16 shows the dissolution curve of composition C (Example 19).

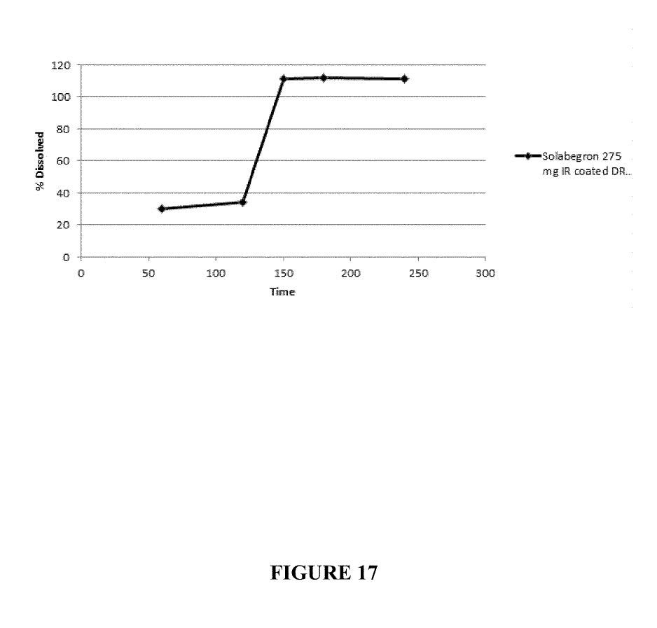

FIG. 17 shows the dissolution curve of composition D (Example 20).

The dissolution studies in Examples 22 and 23 underscore the role surfactants can have in the compositions. Surfactants improve solabegron in vivo dissolution through enhanced dispersion, wetting and solubilization.

The particle size used in these solabegron formulations is ˜4 microns (d90). The surface area to mass ration at that d90 is improved over the same at a d90 of ˜100 microns. In order to take advantage of the improved dissolution of the smaller particle size the solabegron particles must be separated and kept from agglomerating until they have had a chance to go into solution. By intermingling surfactant with solabegron particles these particles are protected at the time of wetting allowing them to disperse avoiding their natural tendency to agglomerate.

Solabegron is hydrophobic. Surfactants in this formulation act to allow an otherwise hydrophobic molecule to closely interface with water.

Surfactants have been demonstrated to dramatically improve the equilibrium solubility of solabegron.

### Example 24

### Pharmacokinetic Concentrations Over 24 Hours of Solabegron Formulations

Pharmacokinetic concentration data from phase I clinical trials was used to evaluate the PK concentration of immediate release solabegron formulations at a single dose of 50 mg, 100 mg or 150 mg respectively and the PK concentration of a single dose 200 mg modified release solabegron formulation (Surfactant Loaded).

| Time (Hrs) | IR-A 50 mg | IR-B 100 mg | IR-C 150 mg | DR 200 mg * |
|---|---|---|---|---|
| 0 | 0.6 | 0.0 | 0.0 | 0.2 |
| 0.5 | 137.5 | 2.8 | 528.0 | 8.2 |
| 1 | 600.1 | 192.0 | 2370.0 | 27.4 |
| 1.5 | 686.2 | 246.0 | 2600.0 | 50.0 |
| 2 | 704.7 | 308.0 | 2405.0 | 60.6 |
| 2.5 | | | | |
| 3 | 590.3 | 299.0 | 2085.0 | 115.6 |
| 3.5 | | | | |
| 4 | 446.4 | 236.0 | 1470.0 | 280.9 |
| 4.5 | | | | |
| 5 | 326.9 | 164.0 | 1380.0 | |
| 5.5 | | | | |
| 6 | 180.0 | 89.1 | 748.5 | 1052.7 |
| 6.5 | | | | |
| 7 | 122.5 | 74.2 | 545.0 | |
| 7.5 | | | | |
| 8 | 92.1 | 69.9 | 410.0 | 763.3 |
| 8.5 | | | | |
| 9 | 70.8 | 58.9 | 346.5 | |
| 9.5 | | | | |
| 10 | 55.6 | 53.5 | 268.0 | |
| 10.5 | | | | |
| 11 | | | | |
| 11.5 | | | | |
| 12 | 38.3 | 33.4 | 191.5 | 281.6 |
| 12.5 | | | | |
| 13 | | | | |
| 13.5 | | | | |
| 14 | 23.3 | 21.5 | 105.2 | |
| 14.5 | | | | |
| 15 | | | | |
| 15.5 | | | | |
| 16 | | | | 118.6 |
| 16.5 | | | | |
| 17 | | | | |
| 17.5 | | | | |
| 18 | 14.5 | 11.2 | 53.0 | 121.6 |
| 18.5 | | | | |
| 19 | | | | |
| 19.5 | | | | |
| 20 | | | | 107.2 |
| 20.5 | | | | |
| 21 | | | | |
| 21.5 | | | | |
| 22 | | | | |
| 22.5 | | | | |
| 23 | | | | |
| 23.5 | | | | |
| 24 | 10.0 | 7.6 | 27.9 | 63.2 |

* DR 200 mg PK data truncated at 24 hour point.

Using the clinical data (above), linear interpolation was performed between the 50 mg and 100 mg immediate release study results to estimate PK concentration profile for an analogous 75 mg immediate release formulation. Similarly linear interpolation was performed between the 100 mg and 150 mg immediate release study results to estimate PK concentration profile for an analogous 125 mg immediate release formulation.

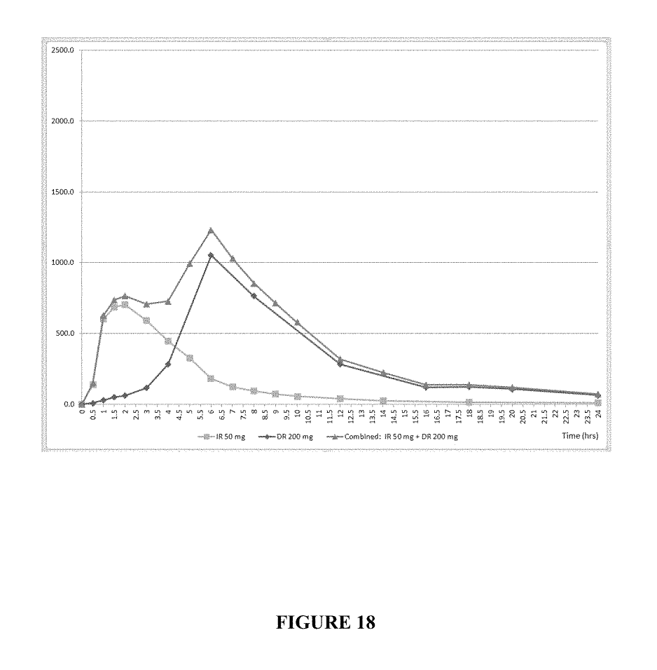

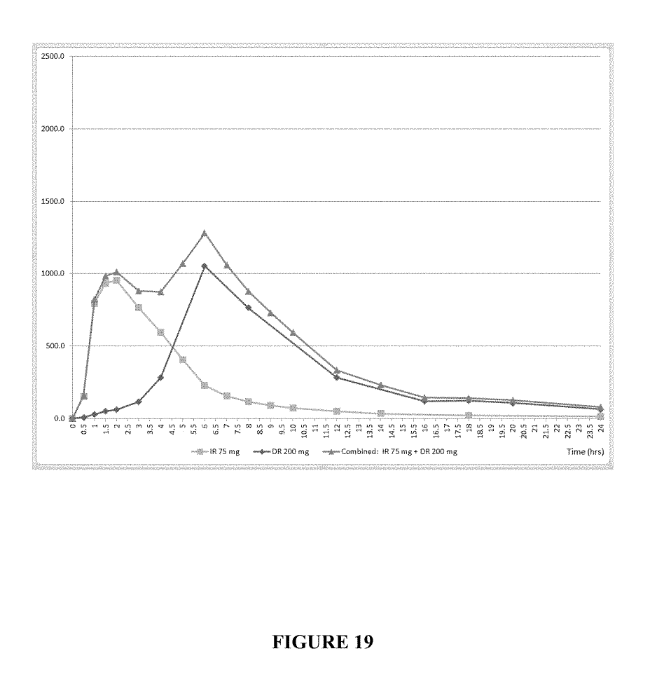

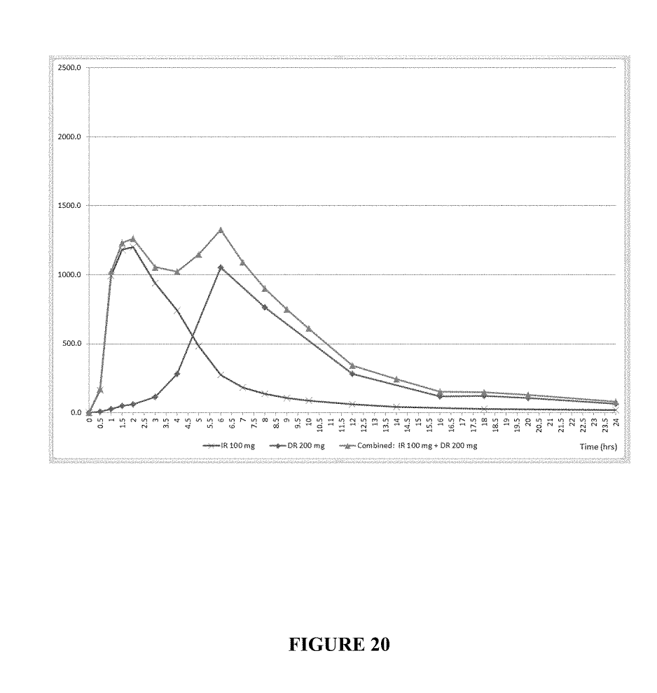

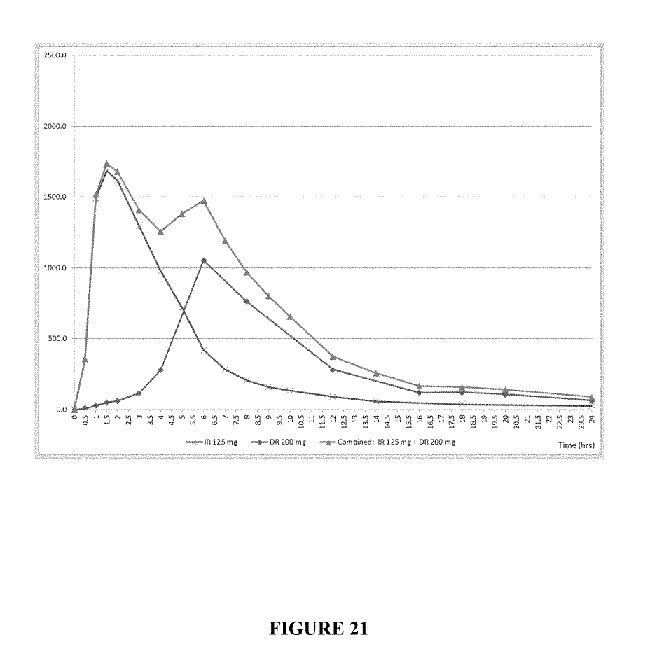

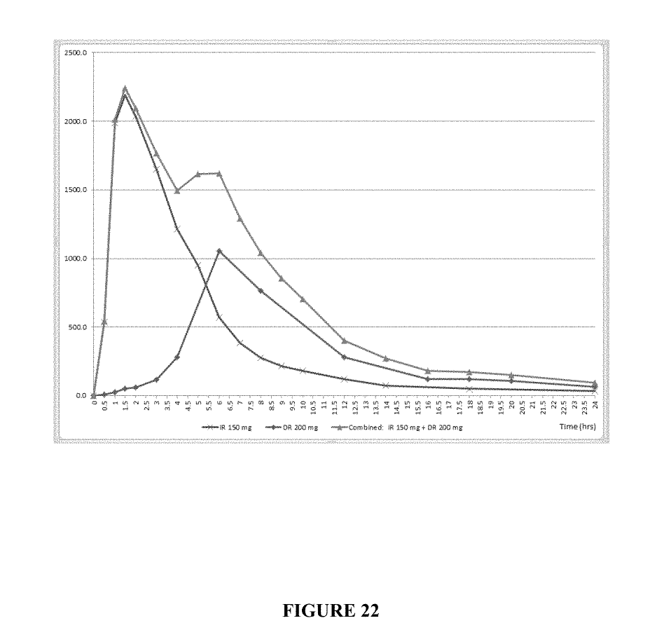

Within all four of the datasets (50 mg, 100 mg and 150 mg immediate release formulations and 200 mg modified release formulation), linear interpolations were performed between actual measurement points (sample times) as necessary to align the different sample times between the two Phase 1 studies to allow different individual PK profiles to be combined. Once all the data points were aligned, the individual immediate release PK concentration profiles were each combined through linear summation with the modified release PK concentration profile to yield estimated immediate release/modified release PK profile data and graphs, as indicated below:
immediate release/modified release PK profile for 50 mg immediate release solabegron combined with 200 mg modified release solabegron (FIG. 18)
immediate release/modified release profile for interpolated 75 mg immediate release solabegron combined with 200 mg modified release solabegron (FIG. 19)
immediate release/modified release profile for 100 mg immediate release solabegron combined with 200 mg modified release solabegron (FIG. 20)
immediate release/modified release profile for interpolated 125 mg immediate release solabegron combined with 200 mg modified release solabegron (FIG. 21)
immediate release/modified release profile for 150 mg immediate release solabegron combined with 200 mg modified release solabegron (FIG. 22)

### Example 25

### Phase 1 Study to Evaluate the Safety, Tolerability and Pharmacokinetics of Candidate Formulations of Orally Administered Solabegron under Fed and Fasted Conditions to Healthy Male

A parallel, single-blind, cross-over study; up to 9 groups of 10 subjects will receive single doses of candidate formulations of solabegron drug products under fasting and fed conditions. The candidate formulations of solabegron drug products differ based in their composition.

Solabegron immediate release and modified release investigational product formulations will be provided as tablets or capsules. Up to 9 formulations of solabegron will be assessed.

All formulations listed are single dosing units except where designated below. IR is immediate/early release and DR is modified or delayed release:
solabegron 200 mg DR, Composition C (Example 19);
solabegron 200 mg DR, Composition A, (Example 17);
solabegron 200 mg DR Composition B (Example 18);
solabegron 200 mg DR total dose, solabegron 2×100 mg DR, Composition E (Example 21);
solabegron 275 mg (75 mg IR/200 mg DR, Composition D, Example 20);
solabegron 75 mg IR (solabegron as a suspension in a geletin capsule imicronized solabegron HCl (about 32.70% w/w/) suspension blended into a suspension with tween 80 (about 33.65%) and propylene glycol (about 33.65% w/w));
solabegron 275 mg total dose (solabegron 75 mg IR (above) and solabegron 200 mg DR (Composition A), given in two separate pharmaceutical compositions).

The objective of this study is to investigate the pharmacokinetics of candidate formulations of solabegron and to investigate the safety and tolerability of candidate formulations of solabegron administered under fasting and fed conditions.

### Pharmacokinetics:

Maximum observed plasma drug concentration (Cₘₐₓ); terminal phase half-life (t_{1/2}); area under the plasma drug concentration versus time curve (AUC₀₋₄, AUC_{0-∞}); clearance (CL/F); volume of distribution (Vd/F); terminal elimination rate constant (λ_{z}); Tmax; other PK parameters may be calculated, as appropriate.

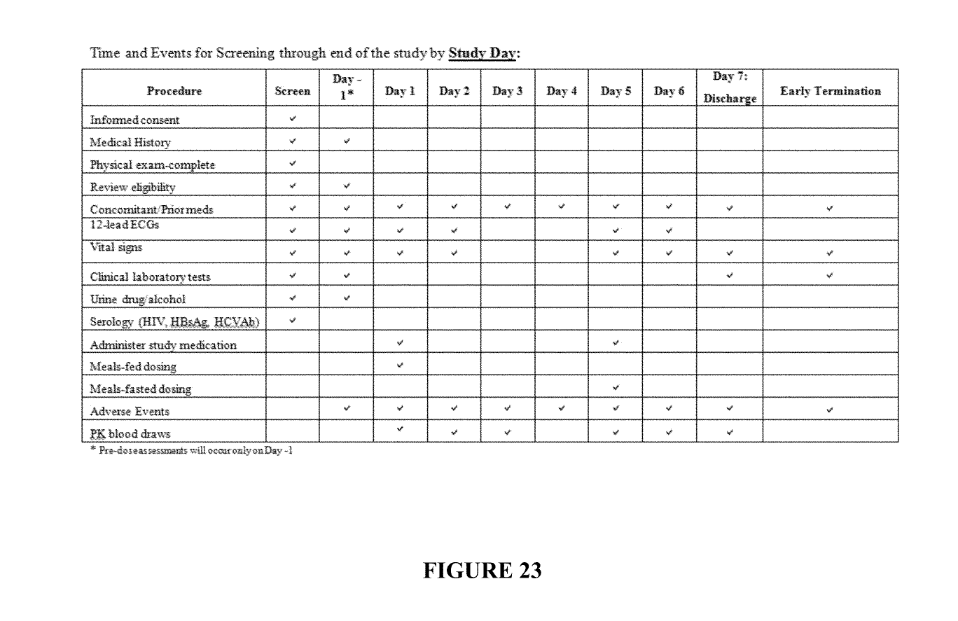

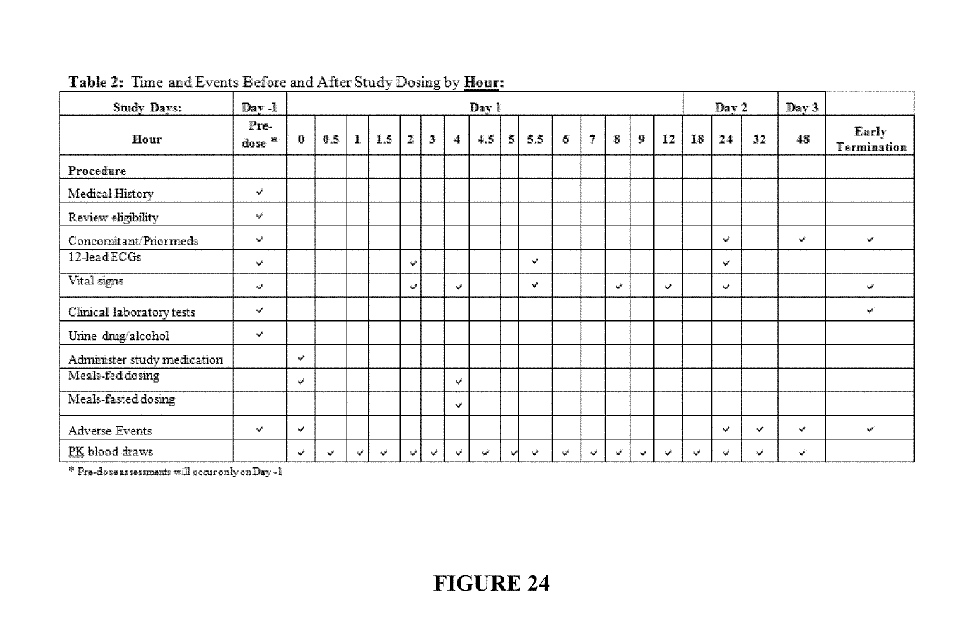

Blood samples (˜4 mL) for the determination of plasma concentrations of solabegron will be collected at the nominal times (±5 min) indicated in the Time and Events Table (FIGS. 23 and 24).

### Treatment Period

Up to 9 groups of 10 subjects each will participate in the study (maximum of 90 subjects). Eligible subjects will be admitted to the research unit on Day −1 to perform pre-dose (baseline) assessments. Some baseline assessments may occur on Day 1 prior to dosing. All assessments and the relative timings for these are listed in the Time and Events Table (FIGS. 23 and 24).

On Day 1, subjects will receive one of the candidate formulations under fed conditions and 96 hours later will receive a second dose of the same study medication under fasted conditions. Subjects will remain confined until approximately 48 hours after their second and final dose.

Subjects who have completed dosing with one of the formulations will be eligible to be dosed with one additional formulation. At least five days should elapse between completion of dosing with the first formulation and the start of dosing with the additional formulation. Subjects will be required to undergo rescreening if more than 30 days have elapsed since the time of their previous discharge.

### Inclusion/Exclusion Criteria

A subject must meet all Inclusion Criteria (not described), and none of the Exclusion Criteria (not described), to participate in this study.

### Fed/Non Fed

For the first dosing period (under fed conditions) and the second dosing period (under fasting conditions), subjects must not have anything to eat a minimum of 8 hours before dosing. For the fed dosing period, following the overnight fast, subjects will have their pre-dose blood sample drawn followed by a standard FDA high fat (50%) breakfast 30 minutes prior to dosing and will be asked to eat the meal in ≦30 minutes. Dosing will begin approximately 30 minutes after the start of the meal on Day 1.

### Dosage and Administration

Subjects will be administered study drug with 240 mL of water under fed and fasting conditions.

### Data Analysis and Statistical Considerations

### Sample Size

Sample size is based on feasibility, and the study is observational in nature. Planned sample size is up to 9 groups of 10 subjects for a maximum of 90 subjects.

### Populations for Analysis

Safety Population: includes all subjects who receive one dose of study medication.

PK Population: includes all subjects who receive a dose of solabegron and have at least one plasma sample obtained and analyzed for solabegron concentration.

### Analyses

Demographics and baseline characteristics will be listed and summarized descriptively by cohort, treatment and overall.

Safety analyses will be performed on data from all subjects in the Safety Population. AEs, clinical laboratory evaluations, and other safety measures (e.g., vital signs, ECGs) will be listed and summarized. No formal statistical analysis of safety data is planned. All available data will be reviewed throughout the study, as the data become available.

Concentration time data for solabegron will be evaluated using standard non-compartmental analysis methods. If feasible, PK parameters at all doses will include Cₘₐₓ, t_{1/2}, AUC₀₋ₜ′ AUC_{0-∞}, Tₘₐₓ, CL/F, Vd/F, and λ_{z}. Other PK parameters will be calculated, as appropriate. Trends in the PK parameters will be evaluated across dose groups. Model-based analyses may be performed following examination of the data.

Pharmacokinetic data will be presented in graphical and/or tabular form and will be summarized descriptively.

Derived PK parameters and plasma concentrations will be listed and summarized descriptively by dose. Descriptive statistics (n, arithmetic mean, standard deviation, 90% confidence interval (CI), minimum, median and maximum) will be calculated for all pharmacokinetic parameters. In addition, for logₑ-transformed PK parameters, geometric mean, 90% CI, and CV % will be provided.

The derived PK parameters will be compared to the derived parameters obtained from healthy volunteers.

Further analysis will be predicated upon review of the data.

### Adverse Events (AE) and Serious Adverse Events (SAE)

An AE is any untoward medical occurrence in a study subject that is temporally associated with the use of a medicinal product, regardless of its potential relationship to the medicinal product. An AE, therefore, can be any unfavorable or unintended sign, including an abnormal laboratory finding, symptom, or disease (new or exacerbated), whether or not related to the study drug.

The definition of an SAE is any untoward medical occurrence that, at any dose: results in death; is life-threatening, requires hospitalization or prolongation of existing hospitalization, results in disability/incapacity, or is a congenital anomaly/birth defect.

All AEs and SAEs will be reported.

### Results

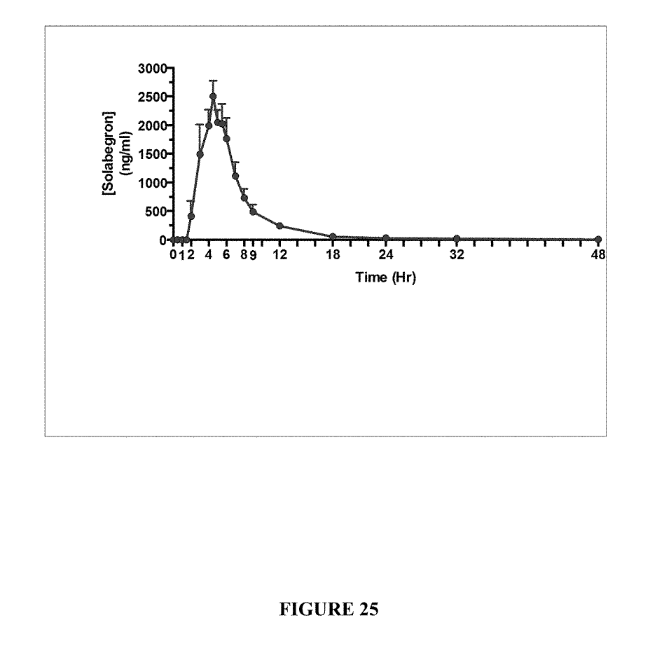

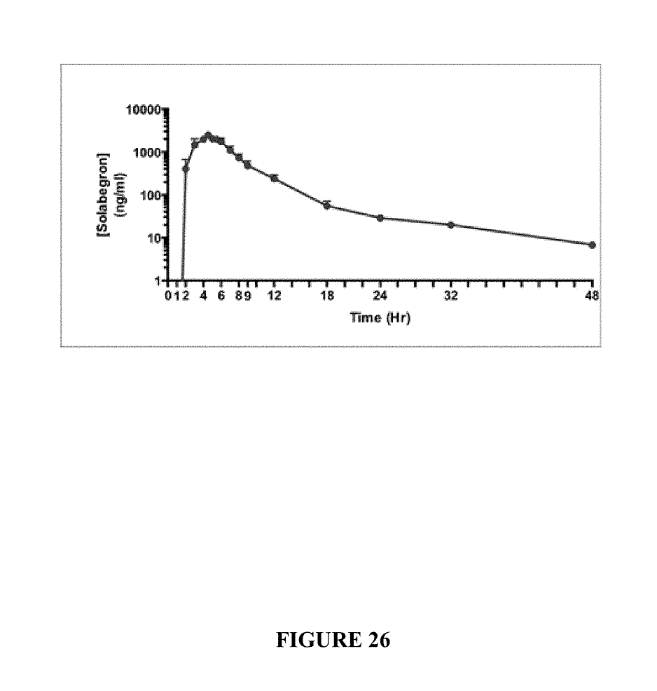

The mean PK data for the Solabegron 200 mg total dose DR, Composition E (Example 21) for the fed arm of 10 patients is shown in FIGS. 25 and 26. This delayed release formulation exhibits the characteristics necessary for the modified release portion of the compositions of the invention. The mean PK parameters for this arm are as follows: AUC 24 hours is about 12,246 ng.hr/mL; AUC 48 hours is about 12, 660 ng.hr/mL; Cₘₐₓ is about 3.14 μg/mL; Tₘₐₓ (time to Cmax) is about 4.10 hours and T_{1/2} is about 5.37 hours.

Although the present disclosure has been described in considerable detail with reference to certain preferred versions thereof, other versions are possible. Therefore, the spirit and scope of the application should not be limited to the description of the preferred versions described herein.

All features disclosed in the specification, including the abstract and drawings, and all the steps in any method or process disclosed, may be combined in any combination, except combinations where at least some of such features and/or steps are mutually exclusive. Each feature disclosed in the specification, including abstract and drawings, can be replaced by alternative features serving the same, equivalent or similar purpose, unless expressly stated otherwise. Thus, unless expressly stated otherwise, each feature disclosed is one example only of a generic series of equivalent or similar features. Various modifications of the application, in addition to those described herein, will be apparent to those skilled in the art from the foregoing description. Such modifications are also intended to fall within the scope of the appended claims.

Throughout the above specification a number of references have been cited and or referred to it is to be understood that unless specifically noted, all references cited in the above specification and or referred to in the above specification are hereby incorporated by reference in their entirety.

What is claimed is:

1. A pharmaceutical composition comprising a therapeutically effective amount of solabegron, wherein the pharmaceutical composition achieves a first target Cₘₐₓ, a second target Cₘₐₓ, a first target Cₘᵢₙ between the first target Cₘₐₓ and the second target Cₘₐₓ, and a second target Cₘᵢₙ after the second target Cₘₐₓ.

2. The pharmaceutical composition of claim 1, wherein said pharmaceutical composition achieves a plasma concentration of about 1 μg/mL or less for about 6 hours to about 9 hours during a twenty-four hour period.

3. The pharmaceutical composition of claim 1, wherein said pharmaceutical composition achieves a target AUC of about 5,000 ng hr/mL to about 30,000 ng hr/mL.

4. The pharmaceutical composition of claim 1, wherein the first target Cₘₐₓ is achieved after the start of a first release of solabegron and the second target Cₘₐₓ is achieved after the start of a second release of solabegron.

5. The pharmaceutical composition according to claim 1, wherein said first target Cₘₐₓ is about 0.5 μg/mL to about 3.5 μg/mL.

6. The pharmaceutical composition according to claim 1, wherein said second target Cₘₐₓ is about 1.5 μg/mL to about 4 μg/mL.

7. The pharmaceutical composition according to claim 1, wherein said first Cₘᵢₙ is about 0.25 μg/mL to about 1.5 μg/mL.

8. The pharmaceutical composition according to claim 1, wherein said second Cₘᵢₙ is about 0.01 μg/mL to about 1.0 μg/mL.

9. The pharmaceutical composition according to claim 1, wherein the time between the first target Cₘₐₓ and the second target Cₘₐₓ is about 2 to about 8 hours.

10. The pharmaceutical composition according to claim 1, wherein the first Cₘᵢₙ is achieved at about 4 to about 8 hours after the first administration.

11. The pharmaceutical composition according to claim 1, wherein the second Cₘᵢₙ is achieved before about 24 hours after administration of the pharmaceutical composition.

12. The pharmaceutical composition according to claim 1, wherein the first target Cₘₐₓ is achieved at about 0.75 to about 4 hours after the first administration.

13. The pharmaceutical composition according to claim 1, wherein the second target Cₘₐₓ is achieved at about 2 to about 8 hours after the first Cₘᵢₙ.

14. The pharmaceutical composition according to claim 4, wherein the first release comprises about 75 mg to about 400 mg of solabegron

15. The pharmaceutical composition according to claim 4, wherein the second release comprises about 100 mg to about 400 mg of solabegron.

16. The pharmaceutical composition according to claim 1, further comprising one or more additional therapeutic agents selected from the group consisting of: antimuscarinic agents; alpha adrenoceptor blockers; botulinum toxin; purinergics; cannabinoids; transient receptor potential (TRP) protein inhibitors; prostaglandins; percutaneous tibial nerve stimulation; 5-alpha reductase inhibitors; and phosphodiesterase-5 inhibitors.

17. A pharmaceutical composition for the delivery of solabegron, comprising
a. at least one immediate release composition, comprising solabegron and at least one pharmaceutically acceptable carrier or diluent; and
b. at least one modified release composition, comprising solabegron and at least one pharmaceutically acceptable carrier or diluent.

18. The pharmaceutical composition according to claim 17, wherein the at least one immediate release composition comprises about 75 mg to about 400 mg solabegron.

19. The pharmaceutical composition according to claim 17, wherein the at least one modified release composition comprises about 100 mg to about 400 mg solabegron.

20. The pharmaceutical composition according to claim 17, wherein the at least one immediate release composition achieves a blood plasma Cₘₐₓ in about 0.75 to about 4 hours after administration to a subject in need of treatment.

21. The pharmaceutical composition according to claim 17, wherein the at least one modified release composition achieves a blood plasma Cₘₐₓ in about 2 to about 8 hours after the first Cₘᵢₙ.

22. The pharmaceutical composition according to claim 17, further comprising one or more additional therapeutic agents or treatments useful for the treatment of LUTS, obesity, type 2 diabetes, heart failure, irritable bowel syndrome and similar gastrointestinal disorders, pre-term labor, depression and anxiety, wherein the one more additional therapeutic agents or treatments are selected from the groups consisting of: antimuscarinic agents; alpha adrenoceptor blockers; botulinum toxin; purinergics; cannabinoids; transient receptor potential (TRP) protein inhibitors; prostaglandins; percutaneous tibial nerve stimulation; 5-alpha reductase inhibitors; and phosphodiesterase-5 inhibitors.

23. The pharmaceutical composition according to claim 22, wherein the one or more additional therapeutic agents may be administered prior to, simultaneously with, or following the administration of the pharmaceutical composition comprising solabegron.

24. The pharmaceutical composition according to claim 17, wherein the at least one immediate release composition achieves a blood plasma Cₘₐₓ from about 0.5 μg/mL to about 3.5 μg/mL.

25. The pharmaceutical composition according to claim 17, wherein the at least one modified release composition achieves a blood plasma Cₘₐₓ from about 1.5 μg/mL to about 4 μg/mL.

26. The pharmaceutical composition according to claim 17, wherein a Cₘᵢₙ from about 0.25 μg/mL to about 1.5 μg/mL is achieved in about 4 to about 8 hours after administration to a subject in need of treatment.

27. The pharmaceutical composition according to claim 17, wherein a Cₘᵢₙ from about 0.01 μg/mL to about 1.0 μg/mL is achieved before about 24 hours after administration to a subject in need of treatment.

28. A method of treating LUTS, obesity, type 2 diabetes, heart failure, irritable bowel syndrome, gastrointestinal disorders, pre-term labor, depression and anxiety in a subject in need thereof, comprising administering to the subject, a pharmaceutical composition for the delivery of solabegron, comprising an immediate release composition, comprising solabegron and at least one pharmaceutically acceptable carrier or diluent; and a modified release composition, comprising solabegron and at least one pharmaceutically acceptable carrier or diluent.

29. The method according to claim 28, wherein the subject achieves a blood plasma Cₘₐₓ of about 0.5 μg/mL to about 3.5 μg/mL in about 0.75 to about 4 hours after administration.

30. The method according to claim 28, wherein the subject achieves a blood plasma Cₘᵢₙ from about 0.25 μg/mL to about 1.5 μg/mL in about 4 to about 8 hours after administration.

31. The method according to claim 28, wherein the subject achieves a blood plasma Cₘₐₓ of about 1.5 μg/mL to about 4 μg/mL in about 2 to about 8 hours after the first Cₘᵢₙ.

32. The method according to claim 28, wherein the subject achieves a blood plasma Cₘᵢₙ from about 0.01 μg/mL to about 1.0 μg/mL before about 24 hours after administration.

33. The method according to claim 28, further comprising administering one or more additional therapeutic agents or treatments useful for the treatment of LUTS, obesity, type 2 diabetes, heart failure, irritable bowel syndrome and similar gastrointestinal disorders, pre-term labor, depression and anxiety, obesity, type 2 diabetes, heart failure, irritable bowel syndrome, gastrointestinal disorders, pre-term labor, depression and anxiety, wherein the one more additional therapeutic agents or treatments are selected from the groups consisting of: antimuscarinic agents; alpha adrenoceptor blockers; botulinum toxin; purinergics; cannabinoids; transient receptor potential (TRP) protein inhibitors; prostaglandins; percutaneous tibial nerve stimulation; 5-alpha reductase inhibitors; and phosphodiesterase-5 inhibitors.

34. The method according to claim 33, wherein the one or more additional therapeutic agents may be administered prior to, simultaneously with, or following the administration of the pharmaceutical composition comprising solabegron.

35. The method according to claim 28, wherein the pharmaceutical composition is administered once a day to a subject in need thereof.

36. The method of claim 28, wherein said LUTS is selected from overactive bladder and a prostate disorder.

37. A method of treating LUTS, obesity, type 2 diabetes, heart failure, irritable bowel syndrome and similar gastrointestinal disorders, pre-term labor, depression and anxiety, in a subject in need thereof, comprising administering to the subject, a pharmaceutical composition, comprising a therapeutically effective amount of solabegron, wherein the pharmaceutical composition releases at least two releases of solabegron, wherein a first release of solabegron achieves a first target Cₘₐₓ, a second release of solabegron achieves a second target Cₘₐₓ, a first target Cₘᵢₙ is achieved between the first release and the second release and a second Cₘᵢₙ is achieved after the second release.

38. The method according to claim 37, wherein said first target Cₘₐₓ is about 0.5 μg/mL to about 3.5 μg/mL.

39. The method according to claim 37, wherein said second target Cₘₐₓ is about 1.5 μg/mL to about 4 μg/mL.

40. The method according to claim 37, wherein said first Cₘᵢₙ is about 0.25 μg/mL to about 1.5 μg/mL.

41. The method according to claim 37, wherein said second Cₘᵢₙ is about 0.01 μg/mL to about 1.0 μg/mL.

42. The method according to claim 37, further comprising administering one or more additional therapeutic agents or treatments useful for the treatment of LUTS, obesity, type 2 diabetes, heart failure, irritable bowel syndrome and similar gastrointestinal disorders, pre-term labor, depression and anxiety, wherein the one more additional therapeutic agents or treatments are selected from the groups consisting of: antimuscarinic agents; alpha adrenoceptor blockers; botulinum toxin; purinergics; cannabinoids; transient receptor potential (TRP) protein inhibitors; prostaglandins; percutaneous tibial nerve stimulation; 5-alpha reductase inhibitors; and phosphodiesterase-5 inhibitors.

43. The method according to claim 42, wherein the one or more additional therapeutic agents may be administered prior to, simultaneously with, or following the administration of the pharmaceutical composition comprising solabegron.

44. The method according to claim 37, wherein the pharmaceutical composition is administered once a day to a subject in need thereof.

45. The method of claim 37, wherein said LUTS is selected from overactive bladder and a prostate disorder.

46. The method of claim 36, wherein treating overactive bladder comprises treating one or more symptoms of OAB selected from the group consisting of: frequency of urinary urgency; nocturia; increase in urinary micturition frequency; and urinary incontinence, voided volume, post-void residual volume and subject reported outcomes.

47. The pharmaceutical composition of claim 1, wherein the solabegron is the amorphous solid form of solabegron.

48. The pharmaceutical composition of claim 47, further comprising two separate and distinct releases of solabegron.

49. The pharmaceutical composition of claim 48, wherein the two release are contained within two separate and distinct drug delivery systems.

50. The pharmaceutical composition of claim 48, wherein the two releases are contained within a single drug delivery system.

51. The pharmaceutical composition of claim 49, wherein the drug delivery system is selected from the group consisting of: tablets; bi-layer tablets; capsules;
multiparticulates; drug coated spheres/pellets; matrix tablets; and multicore tablets.

52. The pharmaceutical composition of claim 50, wherein the drug delivery system is selected from the group consisting of: tablets; bi-layer tablets; capsules;
multiparticulates; drug coated spheres/pellets; matrix tablets; and multicore tablets.

53. A pharmaceutical composition comprising a multiparticulate formulation of mini-tablets each comprising a therapeutically effective amount of solabegron, wherein the pharmaceutical composition achieves a first target Cₘₐₓ, a second target Cₘₐₓ, a first target Cₘᵢₙ between the first target Cₘₐₓ and the second target Cₘₐₓ, and a second target Cₘᵢₙ after the second target Cₘₐₓ.

54. The pharmaceutical composition of claim 53, further comprising at least two separate populations of mini-tablets selected from the group consisting of: immediate release mini-tablets; sustained-release mini-tablets; delayed-release mini-tablets; and modified-release mini-tablets.

55. The pharmaceutical composition of claim 54, wherein the mini-tablets are optionally coated.

56. The pharmaceutical composition of claim 55, wherein the mini-tablets comprise solabegron and at least one pharmaceutically acceptable polymer.

57. The pharmaceutical composition of claim 56, wherein the pharmaceutically acceptable polymer is selected from the group consisting of hydroxypropylmethyl cellulose; polyethylene glycol; hydroxypropyl β-cyclodextrin; polyvinyl pyrrolidine; poloxamer; and hydroxypropyl cellulose.

58. The pharmaceutical composition of claim 57, wherein at least one population of mini-tablets further comprises a controlled-release excipient.

59. The pharmaceutical composition of claim 57, wherein at least one population of mini-tablets further comprises an enteric release coating.

60. The pharmaceutical composition of claim 53, wherein the multiparticulate formulation is contained within a capsule.

61. A pharmaceutical composition comprising a therapeutically effective amount of the amorphous form of solabegron and at least one pharmaceutically acceptable carrier or diluent.

62. The pharmaceutical composition of claim 61, wherein the therapeutically effective amount of amorphous solabegron is about 150 mg to about 850 mg.

63. The pharmaceutical composition of claim 62, wherein the pharmaceutical composition is prepared by spray-drying the therapeutically effective amount of amorphous solabegron with at least one pharmaceutically acceptable polymer.

64. The pharmaceutical composition of claim 63, wherein the pharmaceutically acceptable polymer is selected from the group consisting of hydroxypropylmethyl cellulose; polyethylene glycol; hydroxypropyl β-cyclodextrin; polyvinyl pyrrolidine; poloxamer; and hydroxypropyl cellulose.

65. The pharmaceutical composition of claim 62, wherein the pharmaceutical composition is prepared by hot-melt extruding the therapeutically effective amount of amorphous solabegron with at least one pharmaceutically acceptable polymer.

66. The pharmaceutical composition of claim 65, wherein the pharmaceutically acceptable polymer is selected from the group consisting of hydroxypropylmethyl cellulose; polyethylene glycol; hydroxypropyl β-cyclodextrin; polyvinyl pyrrolidine; poloxamer; and hydroxypropyl cellulose.

67. The method of claim 45, wherein treating overactive bladder comprises treating one or more symptoms of OAB selected from the group consisting of: frequency of urinary urgency; nocturia; increase in urinary micturition frequency; and urinary incontinence, voided volume, post-void residual volume and subject reported outcomes.

68. The method of claim 28, wherein said pharmaceutical composition provides a therapeutically effective amount of solabegron, wherein the pharmaceutical composition achieves a first target Cₘₐₓ, a second target Cₘₐₓ, a first target Cₘᵢₙ between the first target Cₘₐₓ and the second target Cₘₐₓ, and a second target Cₘᵢₙ after the second target Cₘₐₓ.

69. The method of claim 28, wherein the immediate release comprises about 75 mg to about 250 mg of solabegron.

70. The method of claim 28, wherein the modified release comprises about 100 mg to about 400 mg of solabegron.

71. The method of claim 28, wherein the immediate release comprises 50, 55, 60, 65, 75, 80, 85, 90, 95, 100, 105, 110, 115, 120, 125, 130, 135, 140, 145 or 150 mg of solabegron.

72. The method of claim 28, wherein the modified release comprises 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345 or 350 mg of solabegron.

73. The method of claim 28, wherein the immediate release composition is an immediate release drug layer and the modified release composition is a delayed release core, wherein the immediate release drug layer is coated on the delayed release core.

74. The method of claim 28, wherein the pharmaceutical composition is a single unit dose administered once daily.

75. The method of claim 28, wherein the pharmaceutical composition reduces desensitization of the beta-3 adrenoceptor, when compared to an immediate release pharmaceutical composition of solabegron administered twice daily.

76. A method for treating overactive bladder in a patient in need thereof comprising orally administering once a day to the patient a pharmaceutical composition comprising: an immediate release drug layer comprising about 75 mg to about 250 mg solabegron and at least one pharmaceutically acceptable carrier or diluent; and a delayed release core comprising about 100 mg to about 400 mg solabegron and at least one pharmaceutically acceptable carrier or diluent, wherein the immediate release drug layer is coated on the delayed release core.
